(19) United States
(12) Patent Application Publication
(10) Pub. No.: US 2025/0332276 A1
(43) Pub. Date: Oct. 30, 2025

(54) Inhibitors of Molluscum Contagiosum Infection and Methods Using the Same

(71) Applicant: THE TRUSTEES OF THE UNIVERSITY OF PENNSYLVANIA, Philadelphia, PA (US)

(72) Inventors: Robert P. Ricciardi, Kennett Square, PA (US); Hancheng Guan, Downingtown, PA (US); Allen B. Reitz, Chalfont, PA (US); Michael H. Parker, Chalfont, PA (US); Manunya Nuth, Philadelphia, PA (US); Richard W. Scott, Chadds Ford, PA (US); Eric Strobel, Warrington, PA (US)

(21) Appl. No.: 18/868,095

(22) PCT Filed: May 23, 2023
(86) PCT No.: PCT/US2023/067351

§ 371 Date: Nov. 21, 2024

Related U.S. Application Data
(60) Provisional application No. 63/344,874, filed on May 23, 2022.

(51) Int. Cl. A61K 47/64; A61P 31/20; C07D 333/38; C07D 409/12

(52) U.S. Cl. CPC A61K 47/645; A61P 31/20; C07D 333/38; C07D 409/12

(57) ABSTRACT

The present disclosure provides novel compounds, compositions, and methods for treating, preventing, and/or ameliorating an orthopoxvirus infection in a subject in need thereof. In certain embodiments, the orthopoxvirus infection is caused by Molluscum contagiosum.

### CROSS-REFERENCE TO RELATED APPLICATIONS

This application claims priority under 35 U.S.C. § 119(e) to U.S. Provisional Patent Application No. 63/344,874, filed May 23, 2022, which is hereby incorporated by reference in its entirety herein.

### STATEMENT REGARDING FEDERALLY SPONSORED RESEARCH OR DEVELOPMENT

This invention was made with government support under AI125005 and AI162385 awarded by the National Institutes of Health. The government has certain rights in the invention

### BACKGROUND

Molluscum contagiosum (MC) is a skin disease caused by the poxvirus Molluscum contagiosum virus (MCV). MC presents as skin lesions that can last from months to years before resolving. MC lesions occur in the skin of children, adults, and immunosuppressed individuals. MCV is transmitted by direct skin-to-skin contact, sexual contact, auto-inoculation from scratching lesions, and by indirect inoculation from contaminated fomites. The lesions can be painful following treatments intended to reduce spread. The lesions are also psychologically distressful, even more so when they result in scarring. MC occurs in 2-10% of the worldwide population and constitutes about 1% of all diagnosed skin disorders in the U.S., approaching 5% in children. In immunocompromised individuals, this infectious disease can be both severe and protracted. Between 5% and 18% of HIV patients have MC. Often, severe MC disease in AIDS patients begins to resolve while on highly active antiretroviral therapy (HAART). However, there have been documented cases of MC lesions developing soon after starting HAART, suggesting that immune reconstitution inflammatory syndrome (IRIS) might be playing a role in re-emergence of MCV.

The current treatments for MC usually employ physical therapy or chemical agents, which are not uniformly effective or safe, and often fail to completely eliminate lesions and can result in scaring. In addition, the broad-spectrum antiviral drug cidofovir (i.e., 1-((3-hydroxy-2-phosphonyl methoxy)propyl)cytosine), a dCMP analogue, has been used effectively as topical or intravenous medication for MC in immunocompromised patients. However, this drug has side effects including inflammation, erosion, and pain for topical treatment and potential nephrotoxicity for systemic application. To date, no single antiviral therapeutic has been licensed for the specific treatment of MC. The development of such an effective and safe treatment has been hampered mainly by the inability of MCV to propagate in culture.

Processivity factors (PFs) are attractive antiviral therapeutic targets. Their function is to tether DNA polymerases (Pol) to the template to enable synthesis of extended strands. PFs are specific for their cognate DNA Pol and are absolutely essential for DNA synthesis. All DNA Pols from phage to human function with a single cognate PF. However the poxviruses, including the prototypic vaccinia virus (VV) and MCV, are somewhat unusual in that a heterodimer comprising the A20 and D4 viral proteins constitutes the functional PF. D4, which can also function as a uracil-DNA glycosylase repair enzyme, binds to its PF partner A20 but not to E9 Pol. A20 on the other hand, binds to both E9 and D4, suggesting that it serves, in part, as a bridge that indirectly connects D4 to E9.

D4 is also an attractive antiviral target due to its absolute requirement for DNA synthesis by both MCV and VV (the prototypic poxvirus). Notably, in the in vitro DNA synthesis reaction, MCV D4 (mD4) can equally substitute for VV D4 (vD4). This is consistent with mD4 having an amino acid sequence identity of 55% and similarity of 82% to that of VV. Moreover, the virtual 3-D structure of mD4 superimposes onto the known crystal structure of vD4.

There is thus a need in the art for compounds that can be used to treat, prevent, and/or ameliorate MC infections in humans, and methods of use thereof. The present disclosure addresses this need.

### BRIEF SUMMARY

The present disclosure relates, in one aspect, to compounds of Formula (I), or a salt, solvate, enantiomer, diastereomer, geometric isomer, isotopologue, or tautomer thereof:

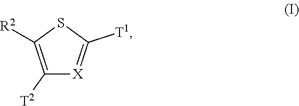

wherein:
T¹ is - - -L²-L¹-R¹ and T² is R³,
   or T¹ is R³ and T² is - - -L²-L¹-R¹;
R¹ is - - -A¹-A²-A³-A⁴.
A¹ is selected from the group consisting of

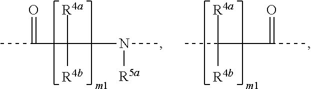

and a bond;
A² is selected from the group consisting of

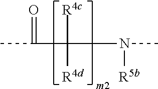

a bond;
A³ is selected from the group consisting of

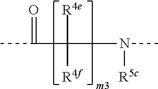

and a bond;
A⁴ is selected from the group consisting of

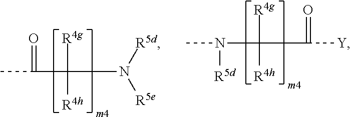

and Y;
   and each of L¹, L², R², R³, R^{4a}, R^{4b}, R^{4c}, R^{4d}, R^{4f}, R^{4h}, R^{5a}, R^{5b}, R^{5c}, R^{5d}, R^{5e}, m1, m2, m3, m4, X, and Y are defined elsewhere herein.

In certain embodiments, the compound of Formula (I) is a compound of Formula (Ia):

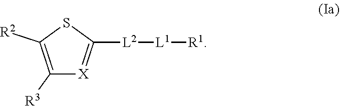

In certain embodiments, the compound of Formula (I) is a compound of Formula (Ib):

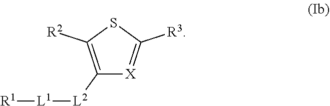

In another aspect, the present disclosure provides a pharmaceutical composition comprising at least one compound of Formula (I) and at least one pharmaceutically acceptable excipient.

In another aspect, the present disclosure provides a method of treating, ameliorating, and/or preventing an orthopoxvirus infection in a subject in need thereof. In certain embodiments, the method comprises administering to the subject a therapeutically effective amount of at least one compound of Formula (I) or the pharmaceutical composition of the present disclosure.

In certain embodiments, the orthopoxvirus infection is caused by a Molluscum contagiosum virus (MCV).

In certain embodiments, the compound of Formula (I) or the pharmaceutical composition is administered topically.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-1B show that D4 is a processivity factor (PF) that serves as a sliding clamp to keep E9 Pol tethered to the DNA template. FIG. 1A: in the absence of mD4, E9 Pol cannot remain bound to the template, preventing it from synthesizing DNA. FIG. 1B: when E9 Pol complexes with D4 (via A20), it remains tethered to the template, enabling it to synthesize extended DNA strands.

FIG. 2 shows that D4 folding and/or function is lost by mutating the GFI region of D4 or by administration of a small molecule inhibitor (i.e., compound 6407).

FIG. 3 provides the chemical structure of compound 7269.

FIG. 4 provides a schematic showing synthesis of a non-limiting variable amino acid-conjugated compound of the present disclosure, wherein: (1) known aminothiophene A is acylated with 2-(4-fluorophenyl)butyric acid to provide amide B; (2) the tert-butyl ester of compound B is deprotected and converted to the corresponding amide with suitable linking moiety (e.g. diaminoethane) to provide compound C; and (3) the terminal amine of compound C is conjugated with any of a number of suitable amino acids, polypeptides, and/or functionalized derivatives thereof to provide compound D.

FIG. 5 provides the chemical structure of compound 9 (I.e., TriValine-7269).

FIGS. 6A-6C show that compound 9 prevents mD4-VV surrogate virus from infecting cells (FIG. 6A) and blocks in vitro processive DNA synthesis by targeting mD4 (FIG. 6B), whereas administration of the TriValine peptide (i.e., (L-Val)₃) alone demonstrated no effect (FIG. 6C); HSV is used as a negative control.

FIG. 7 provides a cell viability dose-response graph showing that compound 9 has no measurable cytotoxicity in the cell viability assay as compared to compound 4 (i.e., MonoValine-7269).

FIG. 8 provides a graph showing quantitative binding of compounds 4, 7, and 9 (K_{D}=2.84 μM) binding to mD4 using microscale thermophoresis.

FIG. 9 provides non-limiting examples of analogues of compound 9, wherein the linker is varied.

FIG. 10 provides a schematic showing the utility of a non-limiting analogue of compound 9 as a prodrug, wherein the linker comprises a sulfenamide moiety, which can be cleaved by glutathione to produce compound 7269.

### DETAILED DESCRIPTION

The present disclosure relates in part to the unexpected discovery of novel inhibitors of Molluscum contagiosum virus (MCV) infection in a human. MCV infects humans only, with the virus infection being confined to the skin and not systemic. In certain embodiments, all the inhibitors described herein also block vaccinia, the prototypic poxvirus. In other embodiments, other poxviruses such as, but not limited to camelpox virus, cowpox virus, ectromelia virus, horsepox virus, monkeypox virus, raccoonpox virus, turkeypoxvirus, variola smallpox virus, Yoka poxvirus, deer poxvirus, fowl poxvirus, myxoma virus, Orf virus, swinepox virus, and Yaba-like disease virus can be inhibited by the compounds described herein.

In certain embodiments, the compounds of the disclosure, or any compositions comprising the same, treat, prevent, and/or ameliorate MCV infection when applied to the skin of an infected human. In yet other embodiments, the compounds of the disclosure, or any compositions comprising the same, are applied to at least one MCV lesion on the skin of the infected human.

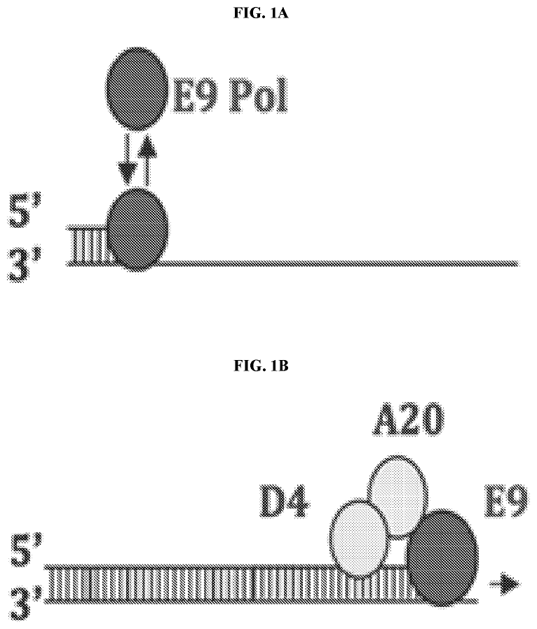

Processivity Factors (PFs) are essential for viral growth. DNA polymerases (Pols) from viruses to mammals fail to synthesize DNA in the absence of PFs. Viral PFs have no cell homologues, making them specific drug targets. Catalytic efficiency of DNA Pols requires that they function processively (i.e., must be capable of incorporate nucleotides continuously without dissociating from the template). Catalytic efficiency of Pols is achieved by associating with their cognate PFs that tether them to the DNA so that the rate of Pol nucleotide incorporation exceeds the rate of Pol dissociation from this template. The tethering of poxvirus E9 Pol to the DNA template by the D4 processivity factor, and the A20 bridging protein, is essential for extended DNA synthesis (FIGS. 1A-1B).

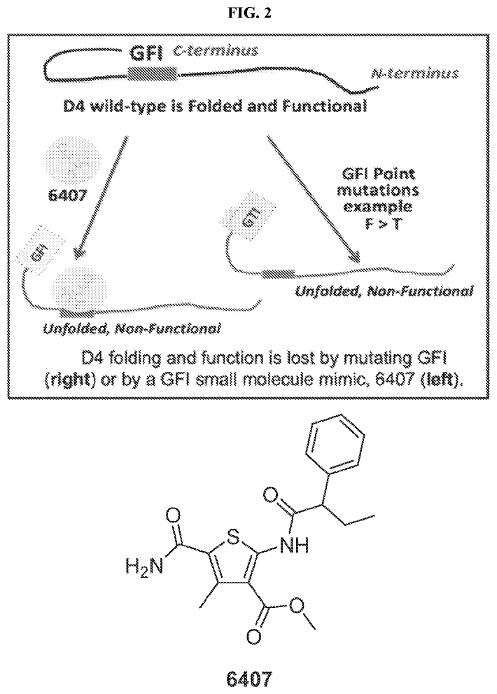

The crystal structure of D4 has been instrumental in deciphering its role in processivity. Mutational and biophysical analyses have revealed 3 amino acids (i.e., GFI) at the C-terminus of D4 which can direct self-folding by contacting amino acid residues located within its inner core. Point mutation of GFI and therapeutic intervention can disrupt D4 folding, and disable processive DNA synthesis (FIG. 2).

U.S. Patent Application No. 63/248,670, which is hereby incorporated by reference in its entirety, describes the identification and development of a class of substituted heterocycles, including substituted thiophenes, which are capable of inhibiting the folding and/or processivity of D4, and accordingly, are suitable for the treatment, prevention, and/or amelioration of orthopoxviruses, including but not limited to Molluscum contagiosum virus (MCV), camelpox virus, cowpox virus, mousepox virus, horsepox virus, monkeypox virus, raccoonpox virus, tanapox virus, variola (smallpox) virus, Yoka poxvirus, cervidpoxvirus (deerpox), avipoxvirus (fowlpox), capripoxvirus (goatpox), leporipoxvirus (myxoma virus), parapoxvirus (orf virus), suipoxvirus (swinepox), and yatapoxvirus (Yaba-like disease virus).

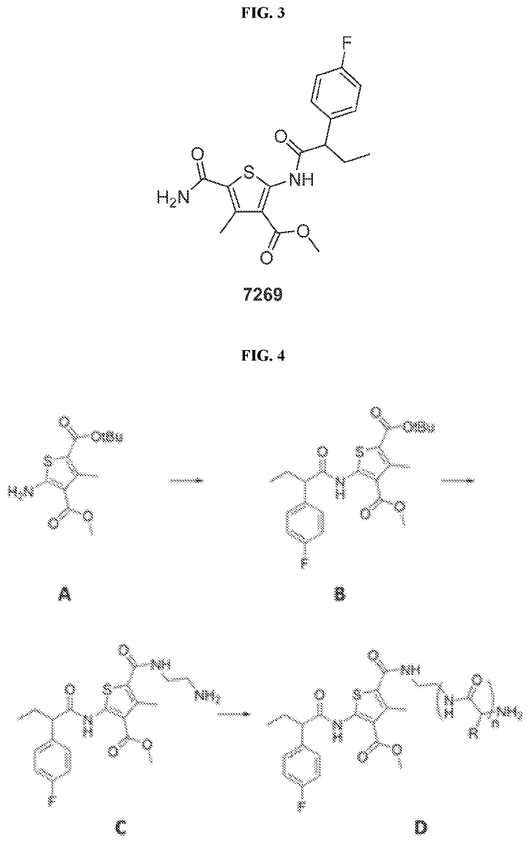

Compound 6407 was shown to block mD4 dependent processive DNA synthesis in vitro (IC₅₀=16.4 μM) and infection of the surrogate virus mD4-VV (EC₅₀=21 μM). While 6407 exhibited specificity in preventing herpes virus from infecting cells, cytotoxicity was observed (CC₅₀=30 μM), accounting for its low Selectivity Index (SI) of <2. The tetra-substituted thiophene of compound 6407 was systematically altered at each of its four positions as well as the central thiophene ring itself, and compound 7269 (FIG. 3) emerged as a mD4 targeted lead with an IC₅₀=6.8 μM and an EC₅₀=13.2 μM and a CC₅₀=103.2 μM. Compound 7269 also exhibited specificity for the surrogate virus mD4-VV. While the SI=7.8 was improved, the CC₅₀ of compound 7269 demonstrated toxicity similar to that of compound 6407.

Thus, the present disclosure relates, in part, to the development of antiviral agents with improved and/or desirable pharmacological properties, including but not limited to lower toxicity, higher bioavailability, and/or higher potency as compared to compounds known in the art.

### Definitions

As used herein, each of the following terms has the meaning associated with it in this section.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, exemplary methods and materials are described.

Generally, the nomenclature used herein and the laboratory procedures in pharmaceutical science and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of 20% or 10%, in certain other embodiments ±5%, in other embodiments ±1%, and in yet other embodiments ±0.1% from the specified value, as such variations are appropriate to perform the disclosed methods.

As used herein, the term “D4” refers to D4 processivity factor. Further, as used herein, the term “mD4” refers to Molluscum D4 processivity factor.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “ED₅₀” or “ED50” refers to the effective dose of a formulation that produces about 50% of the maximal effect in subjects that are administered that formulation.

As used herein, an “effective amount,” “therapeutically effective amount” or “pharmaceutically effective amount” of a compound is that amount of compound that is sufficient to provide a beneficial effect to the subject to which the compound is administered.

“Instructional material,” as that term is used herein, includes a publication, a recording, a diagram, or any other medium of expression that can be used to communicate the usefulness of the composition and/or compound of the disclosure in a kit. The instructional material of the kit may, for example, be affixed to a container that contains the compound and/or composition of the disclosure or be shipped together with a container that contains the compound and/or composition.

As used herein, a “patient” or “subject” can be a human or non-human mammal or a bird. Non-human mammals include, for example, livestock and pets, such as ovine, bovine, porcine, canine, feline and murine mammals. In certain other embodiments, the subject is human.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material can be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it can perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds can also be incorporated into the compositions. The “pharmaceutically acceptable carrier” can further include a pharmaceutically acceptable salt of the compound useful within the disclosure. Other additional ingredients that can be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “pharmaceutical composition” refers to a mixture of at least one compound useful within the disclosure with other chemical components, such as carriers, stabilizers, diluents, dispersing agents, suspending agents, thickening agents, and/or excipients. The pharmaceutical composition facilitates administration of the compound to an organism. Multiple techniques of administering a compound include, but are not limited to, intravenous, oral, aerosol, parenteral, ophthalmic, pulmonary and topical administration.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

The term “solvate,” as used herein, refers to a compound formed by solvation, which is a process of attraction and association of molecules of a solvent with molecules or ions of a solute. As molecules or ions of a solute dissolve in a solvent, they spread out and become surrounded by solvent molecules.

The term “treat,” “treating” or “treatment,” as used herein, means reducing the frequency or severity with which symptoms of a disease or condition are experienced by a subject by virtue of administering an agent or compound to the subject.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkylene” by itself or as part of another substituent means, unless otherwise stated, a straight or branched hydrocarbon group having the number of carbon atoms designated (i.e., C₁-C₁₀ means one to ten carbon atoms) and includes straight, branched chain, or cyclic substituent groups, wherein the group has two open valencies. Examples include methylene, 1,2-ethylene, 1,1-ethylene, 1,1-propylene, 1,2-propylene and 1,3-propylene.

As used herein, the term “cycloalkyl,” by itself or as part of another substituent means, unless otherwise stated, a cyclic chain hydrocarbon having the number of carbon atoms designated (i.e., C3-C6 means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “alkenylene”, employed alone or in combination with other terms, means, unless otherwise stated, a stable mono-unsaturated or di-unsaturated straight chain or branched chain hydrocarbon group having the stated number of carbon atoms wherein the group has two open valencies.

As used herein, the term “alkynylene”, employed alone or in combination with other terms, means, unless otherwise stated, a stable straight chain or branched chain hydrocarbon group with a triple carbon-carbon bond, having the stated number of carbon atoms wherein the group has two open valencies.

As used herein, the term “substituted alkyl”, “substituted cycloalkyl”, “substituted alkenyl”, “substituted alkynyl”, “substituted alkylene”, “substituted alkenylene”, “substituted alkynylene”, “substituted heteroalkyl”, “substituted heteroalkenyl”, “substituted heteroalkynyl”, “substituted aryl”, “substituted heteroaryl” or “substituted heterocycloalkyl” means alkyl, cycloalkyl, alkenyl, alkynyl, alkylene, alkenylene, alkynylene, heteroalkyl, heteroalkenyl, heteroalkynyl, aryl, heteroaryl, or heterocycloalkyl as defined above, substituted by one, two or three substituents selected from the group consisting of C₁-C₁₀ alkyl, halogen, perhaloakyl, ═O, —OH, alkoxy, tetrahydro-2-H-pyranyl, —NH₂, —N(CH₃)₂, phenyl, benzyl, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl. Further, definitions for specific “substituted” moieties can be defined elsewhere herein.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms can be optionally oxidized and the nitrogen heteroatom can be optionally quaternized. The heteroatom(s) can be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms can be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di unsaturated hydrocarbon group consisting of the stated number of carbon atoms and one or two heteroatoms selected from the group consisting of O, N, and S, and wherein the nitrogen and sulfur atoms can optionally be oxidized and the nitrogen heteroatom can optionally be quaternized. Up to two heteroatoms can be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings can be attached together in a pendent manner, such as a biphenyl, or can be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S. and wherein the nitrogen and sulfur heteroatoms can be optionally oxidized, and the nitrogen atom can be optionally quaternized. The heterocyclic system can be attached, unless otherwise stated, at any heteroatom or carbon atom that affords a stable structure. A heterocycle can be aromatic or non-aromatic in nature (e.g., heterocycloalkyl). In certain other embodiments, the heterocycle is a heteroaryl.

As used herein, the term “heteroaryl” or “heteroaromatic” refers to a heterocycle having aromatic character. A polycyclic heteroaryl can include one or more rings that are partially saturated. Examples include tetrahydroquinoline and 2,3 dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. Non-limiting examples of “substituted” groups include C₁-C₁₀ alkyl, halogen, perhaloakyl, ═O, —OH, alkoxy, —NH₂, —N(CH₃)₂, phenyl, benzyl, (1-methyl-imidazol-2-yl), pyridin-2-yl, pyridin-3-yl, pyridin-4-yl, —C(═O)OH, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution can be at any chemically accessible position. In certain other embodiments, the substituents vary in number between one and four. In other embodiments, the substituents vary in number between one and three. In yet other embodiments, the substituents vary in number between one and two. In yet other embodiments, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain can be branched, straight or cyclic, with straight being preferred. The term “substituted heterocycle” and “substituted heteroaryl” as used herein refers to a heterocycle or heteroaryl group having one or more substituents including halogen, CN, OH, NO₂, amino, alkyl, cycloalkyl, carboxyalkyl (C(O)Oalkyl), trifluoroalkyl such as CF₃, aryloxy, alkoxy, aryl, or heteroaryl. A substituted heterocycle or heteroaryl group can have 1, 2, 3, or 4 substituents.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The present disclosure provides a compound of formula (I), or a salt, solvate, enantiomer, diastereomer, geometric isomer, isotopologue, or tautomer thereof:

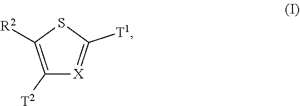

wherein:
T¹ is - - -L²-L¹-R¹ and T² is R³,
   or T¹ is R³ and T² is - - -L²-L¹-R¹;
R¹ is - - -A¹-A²-A³-A⁴;
A¹ is selected from the group consisting of

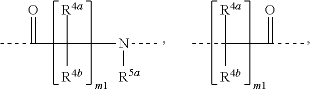

and a bond;
A² is selected from the group consisting of

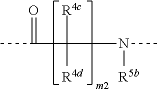

and a bond;
A³ is selected from the group consisting of

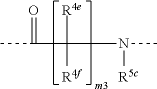

and a bond; and
A⁴ is selected from the group consisting of

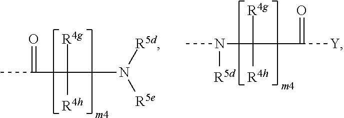

and Y;
L¹ is selected from the group consisting of a bond, —N(R^{a}) (optionally substituted C₁-C₆ alkylenyl)N(R^{b})—, —N(R^{a})S (optionally substituted C₁-C₆ alkylenyl)N(R^{b}), —N(R^{a}) (optionally substituted C₁-C₆ heteroalkylenyl)N(R^{b}), —N(R^{a})S (optionally substituted C₁-C₆ heteroalkylenyl)N(R^{b})—, —N(R^{a}) (optionally substituted C₃-C₈ cycloalkylenyl)N(R^{b}), —N(R^{a})S (optionally substituted C₃-C₈ cycloalkylenyl)N(R^{b})—, —N(R^{a}) (optionally substituted C₂-C₆ heterocyclylenyl)N(R^{b})—, —N(R^{a})S (optionally substituted C₂-C₆ heterocyclylenyl)N(R^{b})—, —N(R^{a}) (optionally substituted C₁-C₆ alkylenyl)C(═O)—, —N(R^{a})S (optionally substituted C₁-C₆ alkylenyl)C(═O)—, optionally substituted C₁-C₆ alkylenyl, —N(R^{a}) (optionally substituted phenylenyl)N(R^{b})—, and —N(R^{a})S (optionally substituted phenylenyl)N(R^{b})—;
L² is selected from the group consisting of —C(═O)—, N(R⁶), and a bond;
Y is selected from the group consisting of N(R^{5e})(R^{5f}) and OR^{5e},
   wherein A¹, A², A³, A⁴, L¹, L², and Y are selected such that:
      a bond between any substituent selected from the group consisting of A¹, A², A³, and A⁴, and any substituent selected from the group consisting of A¹, A², A³, A⁴, L¹, and L², if present, is a C—N bond, and
      a bond between Y and any substituent selected from the group consisting of A¹, A², A³, and A⁴, if present, is a C—N or C—O bond;
R² is selected from the group consisting of N(R^{a})C(═O) (optionally substituted C₁-C₆ alkyl), N(R^{a})C(═O) (optionally substituted C₃-C₈ cycloalkyl), N(C(═O) (optionally substituted C₃-C₈ cycloalkyl))₂, N(R^{a})C(═O)O (optionally substituted phenyl), N(R^{a})C(═O) (optionally substituted C₂-C₈ heterocyclyl), N(R^{a})C(═O)N(R^{b}) (optionally substituted C₁-C₆ alkyl), C(═O)OR^{a}, and C(═O)N(R^{a})C(═O) (optionally substituted C₁-C₆ alkyl);
R³ is selected from the group consisting of H, optionally substituted C₁-C₆ alkyl, optionally substituted phenyl, CN, NO₂, C(═O)OR^{a}, and C(═O)NR^{a}R^{b};
each occurrence of R^{4a}, R^{4b}, R^{4c}, R^{4d}, R^{4e}, R^{4f}, R^{4g}, and R^{4h} is independently selected from the group consisting of H, optionally substituted C₁-C₆ alkyl, optionally substituted C₃-C₆ cycloalkyl, optionally substituted C₂-C₆ heteroalkyl, optionally substituted C₂-C₆ alkenyl, optionally substituted C₂-C₆ alkynyl, optionally substituted heterocyclyl, and optionally substituted phenyl;
each occurrence of R^{5a}, R^{5b}, R^{5c}, R^{5d}, R^{5e}, and R^{5f} is independently selected from the group consisting of H, optionally substituted C₁-C₆ alkyl, optionally substituted C₃-C₈ cycloalkyl, optionally substituted phenyl, C(═O)R^{a}, and C(═O)OR^{a},
   or two vicinal substituents selected from the group consisting of R^{4a}, R^{4b}, R^{4c}, R^{4d}, R^{4e}, R^{4f}, R^{4g}, R^{4h}, R⁴ⁱ, R^{4j}, R^{5a}, R^{5b}, R^{5c}, R^{5d}, R^{5e}, and R^{5f} can optionally combine with the atoms to which they are bound to form an optionally substituted C₄-C₈ heterocycloalkyl;
R⁶ is selected from the group consisting of H and optionally substituted C₁-C₆ alkyl;
X is selected from the group consisting of CR⁶ and N;
m1, m2, m3, and m4 are each independently an integer selected from the group consisting of 1, 2, 3, and 4;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl,
   or geminal R^{a} and R^{b} can optionally combine with the atom to which they are bound to form an optionally substituted C₂-C₈ heterocyclyl.

One skilled in the art would understand that the definitions of A¹, A², and A³ are provided herein without an implied sense of directionality, unless otherwise stated herein or implied from the specific context herein. The same applies to any specific example of A¹, A², and A³, as exemplified elsewhere herein. In a non-limiting example,

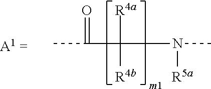

can correspond to either

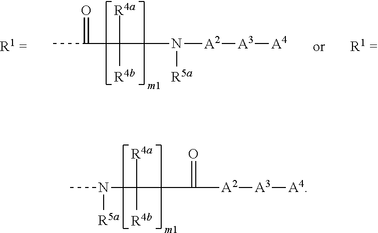

In another non-limiting example,

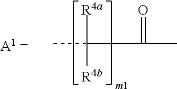

can correspond to either

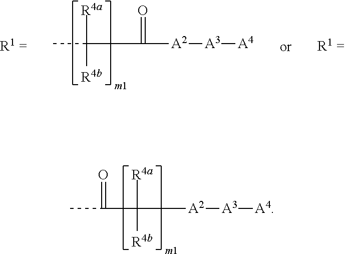

In another non-limiting example,

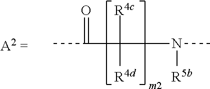

can correspond to either

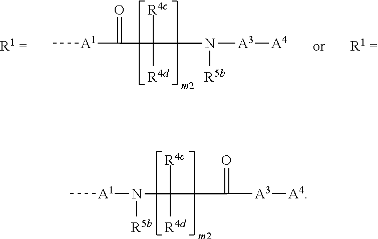

In another non-limiting example,

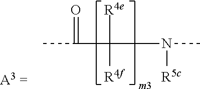

can correspond to either

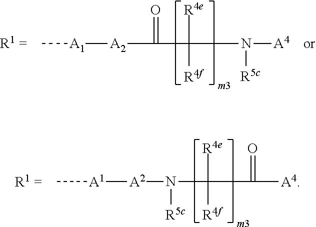

In certain embodiments, the compound of Formula (I) is a compound of Formula (Ia):

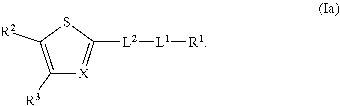

In certain embodiments, the compound of Formula (I) is a compound of Formula (Ib):

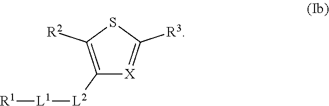

In certain embodiments, each occurrence of optionally substituted alkyl, optionally substituted alkylenyl, optionally substituted cycloalkylenyl, optionally substituted heterocyclylenyl, optionally substituted phenylenyl, optionally substituted cycloalkyl, optionally substituted heteroalkyl, optionally substituted heterocycloalkyl, optionally substituted haloalkyl, optionally substituted alkenyl, and optionally substituted alkynyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OR^{I}, N(R^{I})(R^{II}), SR^{I}, C(═O)R^{I}, C(═O)OR^{I}, OC(═O)OR^{I}, C(═O)N(R^{I})(R^{II}), S(═O)₂N(R^{I})(R^{II}), N(R^{I})C(═O)R^{II}, N(R^{I})C(═NR^{II})N(R^{III})(R^{IV}), N(R^{I})S(═O)₂R^{II}, optionally substituted C₂-C₈ heterocyclyl, and optionally substituted phenyl, wherein each occurrence of R^{I}, R^{II}, R^{III}, and R^{IV} is independently selected from the group consisting of H, C₁-C₆ alkyl, C₃-C₈ cycloalkyl, C₁-C₆ haloalkyl, benzyl, and optionally substituted phenyl.

In certain embodiments, each occurrence of optionally substituted phenyl and optionally substituted heterocyclyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, ORⁱ, N(Rⁱ)(Rⁱⁱ), SRⁱ, C(═O)Rⁱ, C(═O)ORⁱ, OC(═O)ORⁱ, C(═O)N(Rⁱ)(Rⁱⁱ), S(═O)₂N(Rⁱ)(Rⁱⁱ), N(Rⁱ)C(═O)Rⁱⁱ, N(Rⁱ)C(═NRⁱⁱ)N(Rⁱⁱⁱ)(R^{iv}), N(Rⁱ)S(═O)₂Rⁱⁱ, C₂-C₈ heterocyclyl, and phenyl, wherein each occurrence of Rⁱ, Rⁱⁱ, Rⁱⁱⁱ, and R^{iv} is independently selected from the group consisting of H, C₁-C₆ alkyl, C₃-C₈ cycloalkyl, C₁-C₆ haloalkyl, benzyl, and phenyl.

In certain embodiments, A⁴ is

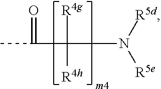

and one of the following applies:
(i) none of A¹, A², and A³ are a bond,
(ii) one of A¹, A², and A³ is a bond,
(iii) two of A¹, A², and A³ are a bond, and
(iv) each of A¹, A², and A³ are a bond.

In certain embodiments, A¹ is

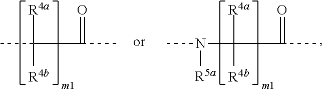

### A⁴ is

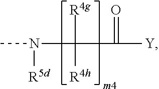

and one of the following applies:
(i) neither of A² and A³ is a bond,
(ii) one of A² and A³ is a bond, and
(iii) both A² and A³ are a bond.

In certain embodiments, A¹ is

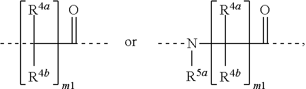

A⁴ is Y, and one of the following applies:
(i) neither of A² and A³ is a bond,
(ii) one of A² and A³ is a bond, and
(iii) both A² and A³ are a bond.

In certain embodiments, A¹ is selected from the group consisting of

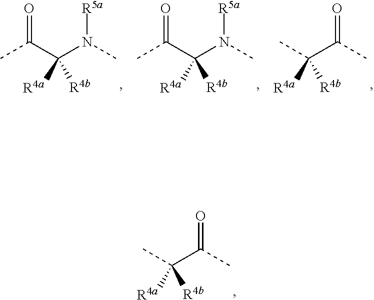

and a bond. In certain embodiments, A¹ is

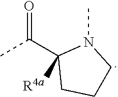

In certain embodiments, A¹ is

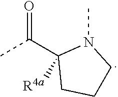

In certain embodiments, A¹ is

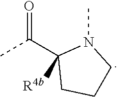

In certain embodiments, A¹ is

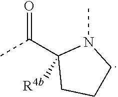

In certain embodiments, A² is selected from the group consisting of

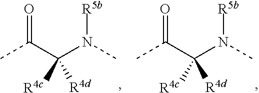

and a bond. In certain embodiments, A² is

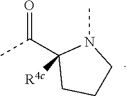

In certain embodiments, A² is

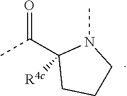

In certain embodiments, A² is

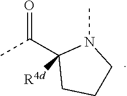

In certain embodiments, A² is

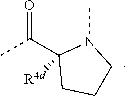

In certain embodiments, A³ is selected from the group consisting of

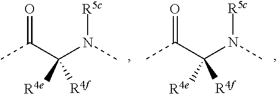

and a bond. In certain embodiments, A³ is

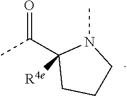

In certain embodiments, A³ is

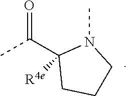

In certain embodiments, A³ is

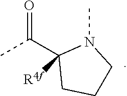

In certain embodiments, A³ is

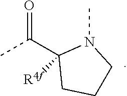

In certain embodiments, A⁴ is selected from the group consisting of

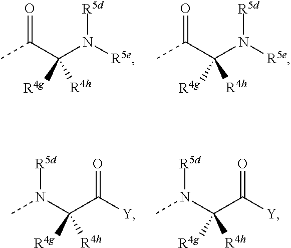

and Y. In certain embodiments. A⁴ is

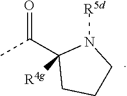

In certain embodiments, A⁴ is

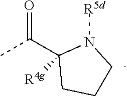

In certain embodiments, A⁴ is

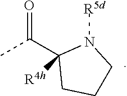

In certain embodiments, A⁴ is

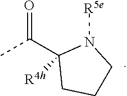

In certain embodiments. A⁴ is

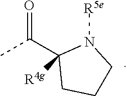

In certain embodiments, A⁴ is

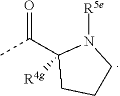

In certain embodiments, A⁴ is

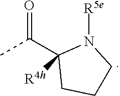

In certain embodiments, A⁴ is

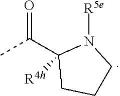

In certain embodiments. A⁴ is

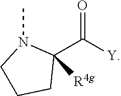

In certain embodiments, A⁴ is

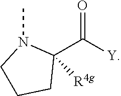

In certain embodiments, A⁴ is

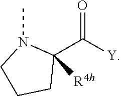

In certain embodiments, A⁴ is

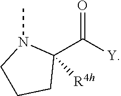

In certain embodiments, R^{4a} is H, methyl. In certain embodiments, R^{4a} is

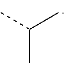

In certain embodiments, R^{4a} is

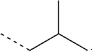

In certain embodiments, R^{4a} is

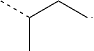

In certain embodiments, R^{4a} is

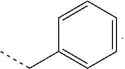

In certain embodiments, R^{4a} is

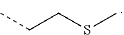

In certain embodiments, R^{4a} is

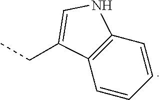

In certain embodiments, R^{4a} is

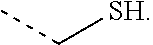

In certain embodiments, R^{4a} is

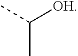

In certain embodiments, R^{4a} is

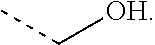

In certain embodiments, R^{4a} is

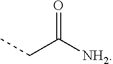

In certain embodiments, R^{4a} is

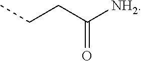

In certain embodiments, R^{4a} is

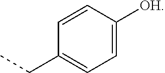

In certain embodiments, R^{4a} is

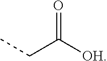

In certain embodiments, R^{4a} is

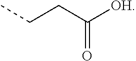

In certain embodiments, R^{4a} is

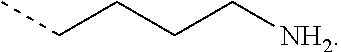

In certain embodiments, R^{4a} is

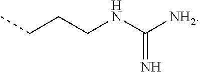

In certain embodiments, R^{4a} is

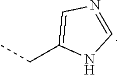

In certain embodiments, R^{4b} is H, methyl. In certain embodiments, R^{4b} is

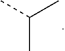

In certain embodiments, R^{4b} is

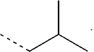

In certain embodiments, R^{4b} is

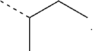

In certain embodiments, R^{4b} is

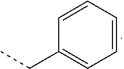

In certain embodiments, R^{4b} is

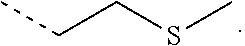

In certain embodiments, R^{4b} is

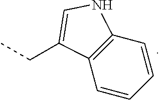

In certain embodiments, R^{4b} is

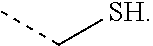

In certain embodiments, R^{4b} is

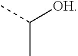

In certain embodiments, R^{4b} is

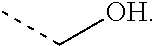

In certain embodiments, R^{4b} is

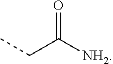

In certain embodiments, R^{4b} is

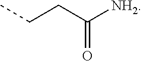

In certain embodiments, R^{4b} is

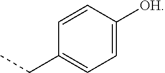

In certain embodiments, R^{4b} is

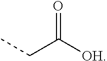

In embodiments, R^{4b} is

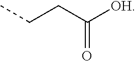

In certain embodiments, R^{4b} is

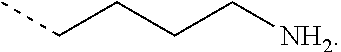

In certain embodiments, R^{4b} is

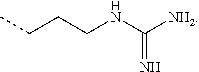

In certain embodiments, R^{4b} is

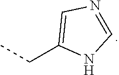

In certain embodiments, R^{4c} is H, methyl. In certain embodiments, R^{4c} is

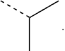

In certain embodiments, R^{4c} is

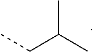

In certain embodiments, R^{4c} is

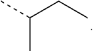

In certain embodiments, R^{4c} is

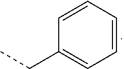

In certain embodiments, R^{4c} is

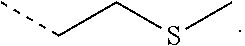

In certain embodiments, R^{4c} is

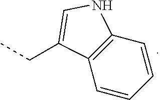

In certain embodiments, R^{4c} is

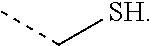

In certain embodiments, R^{4c} is

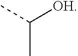

In certain embodiments, R^{4c} is

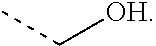

In certain embodiments, R^{4c} is

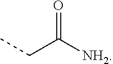

In certain embodiments, R^{4c} is

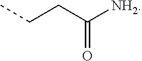

In certain embodiments, R^{4c} is

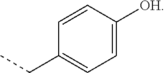

In certain embodiments, R^{4c} is

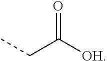

In certain embodiments, R^{4c} is

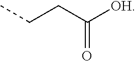

In certain embodiments, R^{4c} is

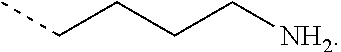

In certain embodiments, R^{4c} is

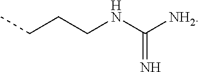

In certain embodiments, R^{4c} is

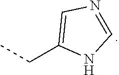

In certain embodiments, R^{4d} is H, methyl. In certain embodiments, R^{4d} is

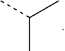

In certain embodiments, R^{4d} is

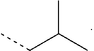

In certain embodiments, R^{4d} is

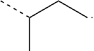

In certain embodiments, R^{4d} is

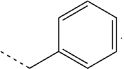

In certain embodiments, R^{4d} is

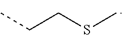

In certain embodiments, R^{4d} is

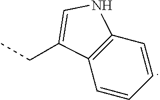

In certain embodiments, R^{4d} is

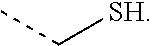

In certain embodiments, R^{4d} is

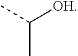

In certain embodiments, R^{4d} is

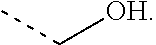

In certain embodiments, R^{4d} is

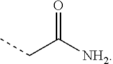

In certain embodiments, R^{4d} is

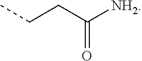

In certain embodiments, R^{4d} is

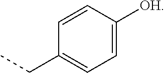

In certain embodiments, R^{4d} is

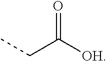

In certain embodiments, R^{4d} is

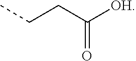

In certain embodiments, R^{4d} is

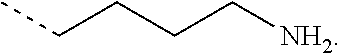

In certain embodiments, R^{4d} is

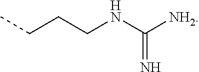

In certain embodiments, R^{4d} is

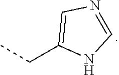

In certain embodiments, R^{4e} is H, methyl. In certain embodiments, R^{4e} is

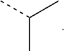

In certain embodiments, R^{4e} is

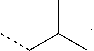

In certain embodiments, R^{4e} is

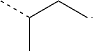

In certain embodiments, R^{4e} is

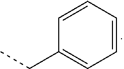

In certain embodiments, R^{4e} is

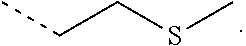

In certain embodiments, R^{4e} is

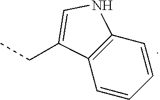

In certain embodiments, R^{4e} is

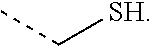

In certain embodiments, R^{4e} is

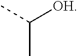

In certain embodiments, R^{4e} is

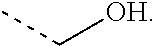

In certain embodiments, R^{4e} is

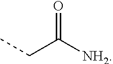

In certain embodiments, R^{4e} is

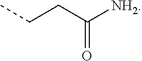

In certain embodiments, R^{4e} is

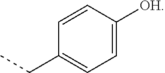

In certain embodiments, R^{4e} is

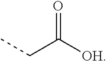

In certain embodiments, R^{4e} is

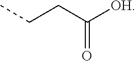

In certain embodiments, R^{4e} is

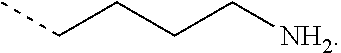

In certain embodiments, R^{4e} is

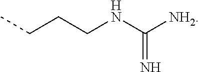

In certain embodiments, R^{4e} is

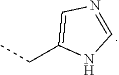

In certain embodiments, R^{4f} is H, methyl. In certain embodiments, R^{4f} is

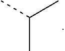

In certain embodiments, R^{4f} is

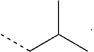

In certain embodiments, R^{4f} is

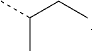

In certain embodiments, R^{4f} is

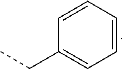

In certain embodiments, R^{4f} is

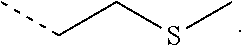

In certain embodiments, R^{4f} is

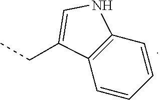

In certain embodiments, R^{4f} is

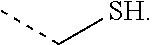

In certain embodiments, R^{4f} is

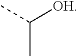

In certain embodiments, R^{4f} is

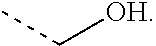

In certain embodiments, R^{4f} is

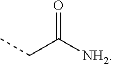

In certain embodiments, R^{4f} is

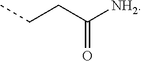

In certain embodiments, R^{4f} is

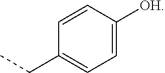

In certain embodiments, R^{4f} is

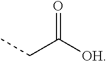

In certain embodiments, R^{4f} is

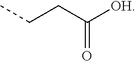

In certain embodiments, R^{4f} is

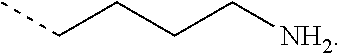

In certain embodiments, R^{4f} is

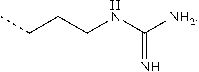

In certain embodiments, R^{4f} is

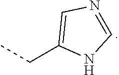

In certain embodiments, R^{4g} is H, methyl. In certain embodiments, R^{4g} is

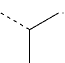

In certain embodiments, R^{4g} is

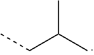

In certain embodiments, R^{4g} is

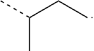

In certain embodiments, R^{4g} is

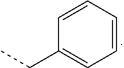

In certain embodiments, R^{4g} is

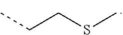

In certain embodiments, R^{4g} is

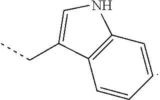

In certain embodiments, R^{4g} is

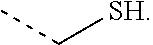

In certain embodiments, R^{4g} is

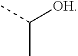

In certain embodiments, R^{4g} is

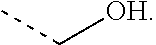

In certain embodiments, R^{4g} is

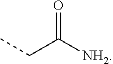

In certain embodiments, R^{4g} is

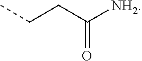

In certain embodiments, R^{4g} is

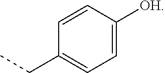

In certain embodiments, R^{4g} is

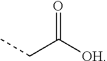

In certain embodiments, R^{4g} is

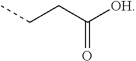

In certain embodiments, R^{4g} is

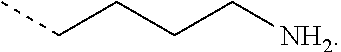

In certain embodiments, R^{4g} is

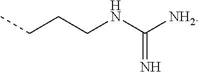

In certain embodiments, R^{4g} is

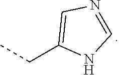

In certain embodiments, R^{4h} is H, methyl. In certain embodiments, R^{4h} is

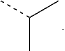

In certain embodiments, R^{4h} is

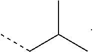

In certain embodiments, R^{4h} is

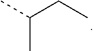

In certain embodiments, R^{4h} is

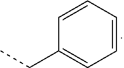

In certain embodiments, R^{4h} is

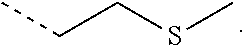

In certain embodiments, R^{4h} is

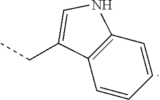

In certain embodiments, R^{4h} is

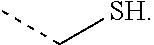

In certain embodiments, R^{4h} is

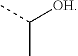

In certain embodiments, R^{4h} is

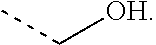

In certain embodiments, R^{4h} is

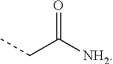

In certain embodiments, R^{4h} is

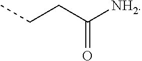

In certain embodiments, R^{4h} is

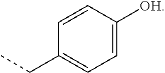

In certain embodiments, R^{4h} is

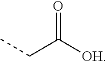

In certain embodiments, R^{4h} is

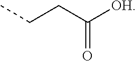

In certain embodiments, R^{4h} is

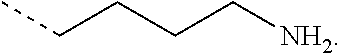

In certain embodiments, R^{4h} is

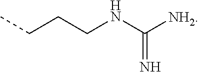

In certain embodiments, R^{4h} is

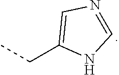

In certain embodiments, at least one of R^{4a} and R^{4b} is H. In certain embodiments, at least one of R^{4c} and R^{4d} is H. In certain embodiments, at least one of R^{4e} and R^{4f} is H. In certain embodiments, at least one of R^{4g} and R^{4h} is H.

In certain embodiments, R^{5a} is H. In certain embodiments, R^{5b} is H. In certain embodiments, R^{5c} is H. In certain embodiments, R^{5d} is H. In certain embodiments, R^{5e} is H. In certain embodiments, R^{5e} is C(═O)O(C(CH₃)₃). In certain embodiments, R^{5f} is H. In certain embodiments, R^{5f} is C(═O)O(C(CH₃)₃).

In certain embodiments, A¹ is

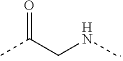

In certain embodiments, A¹ is

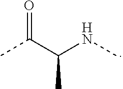

In certain embodiments, A¹ is

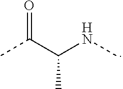

In certain embodiments, A¹ is

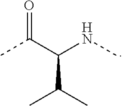

In certain embodiments, A¹ is

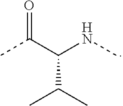

In certain embodiments, A¹ is

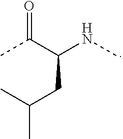

In certain embodiments, A¹ is

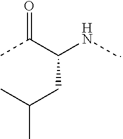

In certain embodiments, A² is

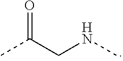

In certain embodiments, A² is

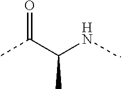

In certain embodiments, A² is

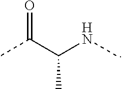

In certain embodiments, A² is

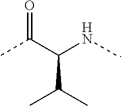

In certain embodiments, A² is

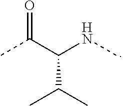

In certain embodiments, A² is

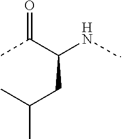

In certain embodiments, A² is

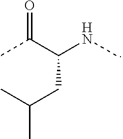

In certain embodiments, A³ is

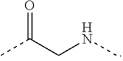

In certain embodiments, A³ is

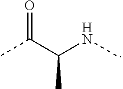

In certain embodiments, A³ is

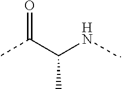

In certain embodiments, A³ is

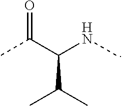

In certain embodiments, A³ is

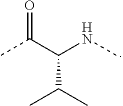

In certain embodiments, A³ is

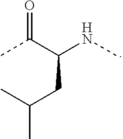

In certain embodiments, A³ is

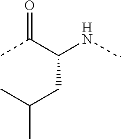

In certain embodiments, A⁴ is

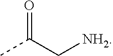

In certain embodiments, A⁴ is

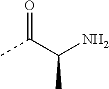

In certain embodiments, A⁴ is

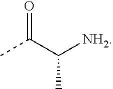

In certain embodiments, A⁴ is

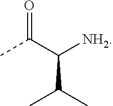

In certain embodiments, A⁴ is

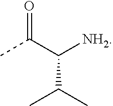

In certain embodiments, A⁴ is

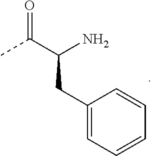

In certain embodiments, A⁴ is

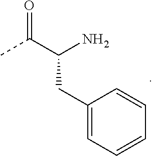

In certain embodiments A⁴ is

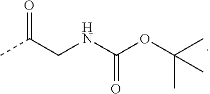

In certain embodiments, A⁴ is

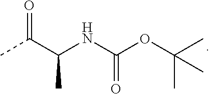

In certain embodiments, A⁴ is

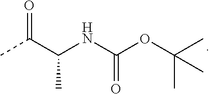

In certain embodiments, A⁴ is

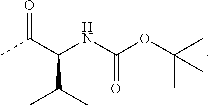

In certain embodiments A⁴ is

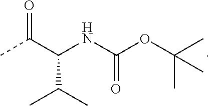

In certain embodiments, R¹ is NH₂. In certain embodiments, R¹ is Ot-Bu. In certain embodiments, R¹ is

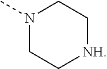

In certain embodiments, R¹ is

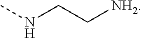

In certain embodiments, R¹ is

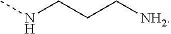

In certain embodiments, R¹ is

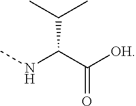

In certain embodiments, R¹ is

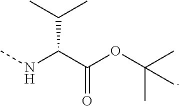

In certain embodiments, R¹ is

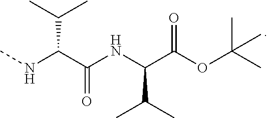

In certain embodiments, R¹ is

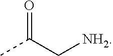

In certain embodiments, R¹ is

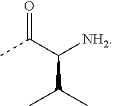

In certain embodiments, R¹ is

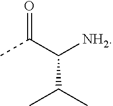

In certain embodiments, R¹ is

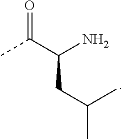

In certain embodiments, R¹ is

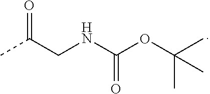

In certain embodiments, R¹ is

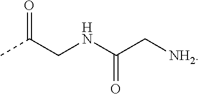

In certain embodiments, R¹ is

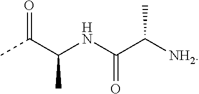

In certain embodiments, R¹ is

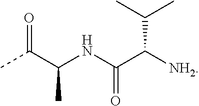

In certain embodiments, R¹ is

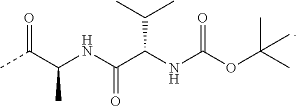

In certain embodiments, R¹

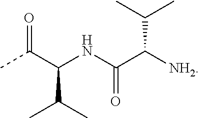

In certain embodiments, R¹ is

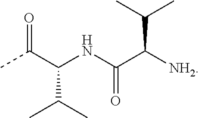

In certain embodiments, R¹ is

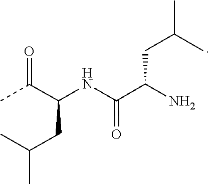

In certain embodiments, R¹ is

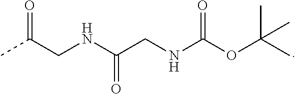

In certain embodiments, R¹ is

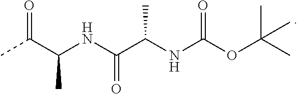

In certain embodiments, R¹ is

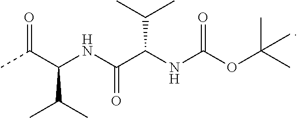

In certain embodiments, R¹ is

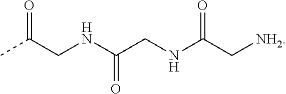

In certain embodiments, R¹ is

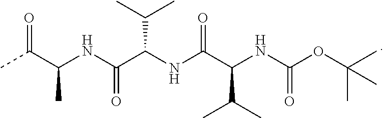

In certain embodiments, R¹ is

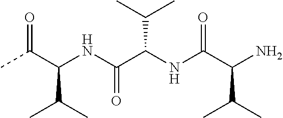

In certain embodiments, R¹ is

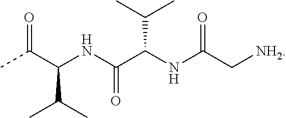

In certain embodiments, R¹ is

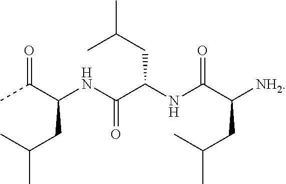

In certain embodiments, R¹ is

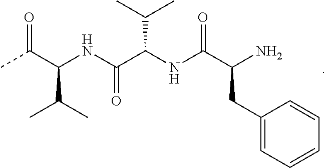

In certain embodiments, R¹ is

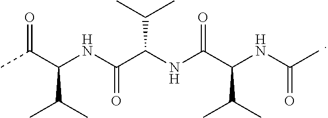

In certain embodiments, R¹ is

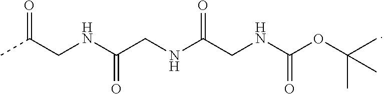

In certain embodiments, R¹ is

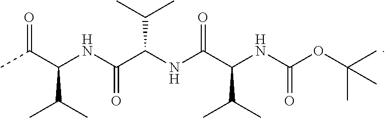

In certain embodiments, R¹ is

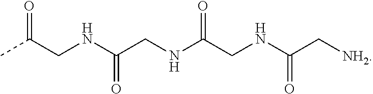

In certain embodiments, R¹ is

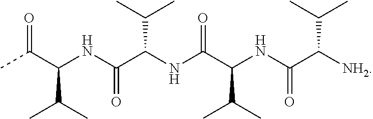

In certain embodiments, R¹ is

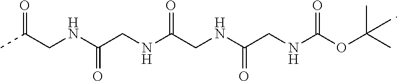

In certain embodiments, R¹ is

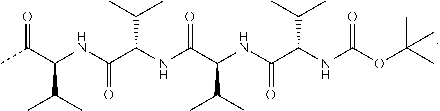

In certain embodiments, R² is

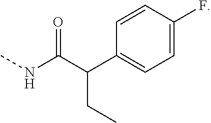

In certain embodiments, R² is

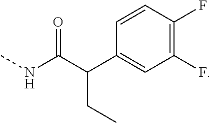

In certain embodiments, R² is

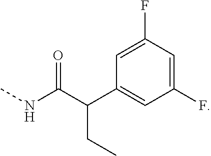

In certain embodiments, R² is

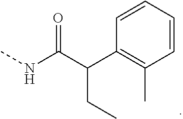

In certain embodiments, R² is

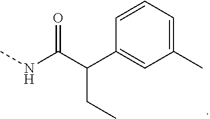

In certain embodiments, R² is

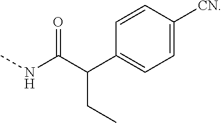

In certain embodiments, R² is

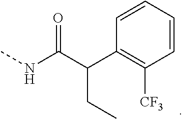

In certain embodiments, R² is

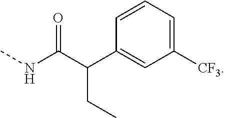

In certain embodiments, R² is

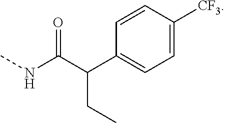

In certain embodiments, R² is

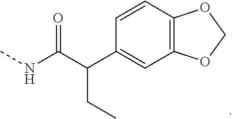

In certain embodiments, R³ is H.

In certain embodiments, L¹ is selected from the group consisting of

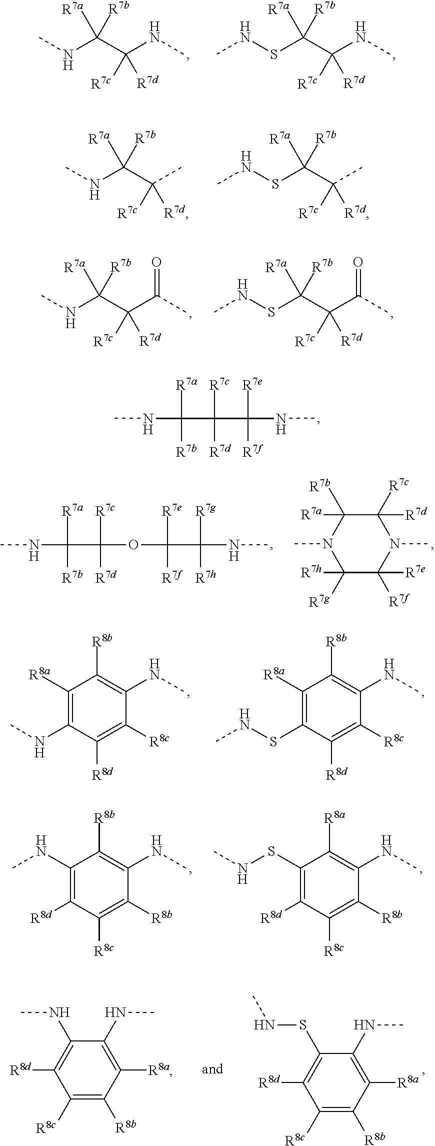

wherein:
R^{7a}, R^{7b}, R^{7c}, R^{7d}, R^{7e}, R^{7f}, R^{7g} and R^{7h} are each independently selected from the group consisting of H and C₁-C₆ alkyl; and
R^{8a}, R^{8b}, R^{8c}, and R^{8d} are each independently selected from the group consisting of H, C₁-C₆ alkyl, C₁-C₆ alkoxy, C₁-C₃ haloalkyl, C₁-C₆ alkoxy, halogen, CN, and NO₂.

In certain embodiments, at least one of R^{7a}, R^{7b}, R^{7c}, R^{7d}, R^{7e}, R^{7f}, R^{7g}, and R^{7h} is H. In certain embodiments, at least two of R^{7a}, R^{7b}, R^{7c}, R^{7d}, R^{7e}, R^{7f}, R^{7g}, and R^{7h} are H. In certain embodiments, at least three of R^{7a}, R^{7b}, R^{7c}, R^{7d}, R^{7e}, R^{7f}, R^{7g}, and R^{7h} are H. In certain embodiments, at least four of R^{7a}, R^{7b}, R^{7c}, R^{7d}, R^{7e}, R^{7f}, R^{7g}, and R^{7h} are H. In certain embodiments, at least five of R^{7a}, R^{7b}, R^{7c}, R^{7d}, R^{7e}, R^{7f}, R^{7g}, and R^{7h} are H. In certain embodiments, at least six of R^{7a}, R^{7b}, R^{7c}, R^{7d}, R^{7e}, R^{7f}, R^{7g}, and R^{7h} are H. In certain embodiments, at least seven of R^{7a}, R^{7b}, R^{7c}, R^{7d}, R^{7e}, R^{7f}, R^{7g}, and R^{7h} are H. In certain embodiments, each of R^{7a}, R^{7b}, R^{7c}, R^{7d}, R^{7e}, R^{7f}, R^{7g}, and R^{7h} are H.

In certain embodiments, at least one of R^{8a}, R^{8b}, R^{8c}, and R^{8d} is H. In certain embodiments, at least two of R^{8a}, R^{8b}, R^{8c}, and R^{8d} are H. In certain embodiments, at least three of R^{8a}, R^{8b}, R^{8c}, and R^{8d} are H. In certain embodiments, each of R^{8a}, R^{8b}, R^{8c}, and R^{8d} are H.

In certain embodiments, L¹ is

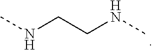

In certain embodiments, L¹ is

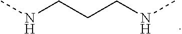

In certain embodiments, L¹ is

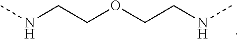

In certain embodiments, L¹ is

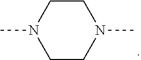

In certain embodiments, L² is —C(═O)—.

In certain embodiments, X is C(CH₃).

In certain embodiments, the compound is selected from the group consisting of:
methyl 5-((2-(2-((tert-butoxycarbonyl)amino)acetamido)ethyl)carbamoyl)-2-(2-(4-fluorophenyl)butanamido)-4-methylthiophene-3-carboxylate;
methyl 5-((2-(2-aminoacetamido)ethyl)carbamoyl)-2-(2-(4-fluorophenyl)butanamido)-4-methylthiophene-3-carboxylate;
methyl 5-((2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate;
methyl 5-((2-((S)-2-amino-3-methylbutanamido)ethyl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate;
methyl 5-((2-(2-(2-aminoacetamido)acetamido)ethyl)carbamoyl)-2-(2-(4-fluorophenyl)butanamido)-4-methylthiophene-3-carboxylate;
methyl 5-((2-((S)-2-((S)-2-amino-3-methylbutanamido)-3-methylbutanamido)ethyl)carbamoyl)-2-(2-(4-fluorophenyl)butanamido)-4-methylthiophene-3-carboxylate;
methyl 2-(2-(4-fluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate;
methyl 5-(((5S,8S,11S,14S)-14-amino-5,8,11-triisopropyl-15-methyl-4,7,10,13-tetraoxo-3,6,9,12-tetraazahexadecyl)carbamoyl)-2-(2-(4-fluorophenyl)butanamido)-4-methylthiophene-3-carboxylate;
methyl 5-((2,2-dimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate;
methyl 5-((2-(2-(2-(2-aminoacetamido)acetamido)acetamido)ethyl)carbamoyl)-2-(2-(4-fluorophenyl)butanamido)-4-methylthiophene-3-carboxylate:
methyl 5-((2,2-dimethyl-4,7,10,13,16-pentaoxo-3-oxa-5,8,11,14,17-pentaazanonadecan-19-yl)carbamoyl)-2-(2-(4-fluorophenyl)butanamido)-4-methylthiophene-3-carboxylate;
methyl 5-((14-amino-4,7,10,13-tetraoxo-3,6,9,12-tetraazatetradecyl)carbamoyl)-2-(2-(4-fluorophenyl)butanamido)-4-methylthiophene-3-carboxylate;
methyl 5-((2-((S)-2-amino-4-methylpentanamido)ethyl)carbamoyl)-2-(2-(4-fluorophenyl)butanamido)-4-methylthiophene-3-carboxylate;
methyl 5-((2-((S)-2-((S)-2-amino-4-methylpentanamido)-4-methylpentanamido)ethyl)carbamoyl)-2-(2-(4-fluorophenyl)butanamido)-4-methylthiophene-3-carboxylate;
methyl 5-((2-((S)-2-((S)-2-((S)-2-amino-4-methylpentanamido)-4-methylpentanamido)-4-methylpentanamido)ethyl)carbamoyl)-2-(2-(4-fluorophenyl)butanamido)-4-methylthiophene-3-carboxylate;
methyl 5-((2-((R)-2-amino-3-methylbutanamido)ethyl)carbamoyl)-2-(2-(4-fluorophenyl)butanamido)-4-methylthiophene-3-carboxylate;
methyl 5-((2-((R)-2-((R)-2-amino-3-methylbutanamido)-3-methylbutanamido)ethyl)carbamoyl)-2-(2-(4-fluorophenyl)butanamido)-4-methylthiophene-3-carboxylate;
methyl 5-((2-((R)-2-((R)-2-((R)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate;
methyl 5-((2-((S)-2-((S)-2-((S)-2-amino-3-phenylpropanamido)-3-methylbutanamido)-3-methylbutanamido)ethyl)carbamoyl)-2-(2-(4-fluorophenyl)butanamido)-4-methylthiophene-3-carboxylate;
methyl 5-((2-((S)-2-((S)-2-(2-aminoacetamido)-3-methylbutanamido)-3-methylbutanamido)ethyl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3-oxa-5,8,11-triazatridecan-13-yl)carbamoyl)-4-methyl-2-(2-(4-(trifluoromethyl)phenyl)butanamido)thiophene-3-carboxylate;
methyl 5-((2-((S)-2-((S)-2-amino-3-methylbutanamido)-3-methylbutanamido)ethyl)carbamoyl)-4-methyl-2-(2-(4-(trifluoromethyl)phenyl)butanamido)thiophene-3-carboxylate;
methyl 4-methyl-2-(2-(4-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 4-methyl-5-(((6S,9S,12S,15S)-6,9,12,15-tetraisopropyl-2,2-dimethyl-4,7,10,13,16-pentaoxo-3-oxa-5,8,11,14,17-pentaazanonadecan-19-yl)carbamoyl)-2-(2-(4-(trifluoromethyl)phenyl)butanamido)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(4-(trifluoromethyl)phenyl)butanamido)thiophene-3-carboxylate;
methyl 5-(((5S,8S,11S,14S)-14-amino-5,8,11-triisopropyl-15-methyl-4,7,10,13-tetraoxo-3,6,9,12-tetraazahexadecyl)carbamoyl)-4-methyl-2-(2-(4-(trifluoromethyl)phenyl)butanamido)thiophene-3-carboxylate;
methyl 4-methyl-5-(((6S,9S)-2,2,6,9-tetramethyl-4,7,10-trioxo-3-oxa-5,8,11-triazatridecan-13-yl)carbamoyl)-2-(2-(4-(trifluoromethyl)phenyl)butanamido)thiophene-3-carboxylate;
methyl 5-((2-((S)-2-((S)-2-aminopropanamido)propanamido)ethyl)carbamoyl)-4-methyl-2-(2-(4-(trifluoromethyl)phenyl)butanamido)thiophene-3-carboxylate;
methyl 2-(2-(4-fluorophenyl)butanamido)-4-methyl-5-(piperazine-1-carbonyl)thiophene-3-carboxylate;
tert-butyl (2-(5-carbamoyl-2-(2-(4-fluorophenyl)butanamido)-4-methylthiophene-3-carboxamido)ethyl)carbamate;
methyl 5-(4-((tert-butoxycarbonyl)-L-valyl-L-valyl)piperazine-1-carbonyl)-2-(2-(4-fluorophenyl)butanamido)-4-methylthiophene-3-carboxylate;
methyl 5-(4-(L-valyl-L-valyl)piperazine-1-carbonyl)-2-(2-(4-fluorophenyl)butanamido)-4-methylthiophene-3-carboxylate;
N⁴-(2-aminoethyl)-5-(2-(4-fluorophenyl)butanamido)-3-methylthiophene-2,4-dicarboxamide;
tert-butyl ((2S)-1-(((2S)-1-((2-(5-carbamoyl-2-(2-(4-fluorophenyl)butanamido)-4-methylthiophene-3-carboxamido)ethyl)amino)-3-methyl-1-oxobutan-2-yl)amino)-3-methyl-1-oxobutan-2-yl)carbamate;
N⁴-(2-((S)-2-((S)-2-amino-3-methylbutanamido)-3-methylbutanamido)ethyl)-5-(2-(4-fluorophenyl)butanamido)-3-methylthiophene-2,4-dicarboxamide;
methyl 2-(2-(4-fluorophenyl)butanamido)-4-methyl-5-(((5S,8S,11S)-5,8,11-triisopropyl-4,7,10,13-tetraoxo-3,6,9,12-tetraazatetradecyl)carbamoyl)thiophene-3-carboxylate;
methyl 5-(4-(L-valyl-L-valyl-L-valyl)piperazine-1-carbonyl)-2-(2-(4-fluorophenyl)butanamido)-4-methylthiophene-3-carboxylate;
tert-butyl ((7S,10S,13S)-1-(5-carbamoyl-2-(2-(4-fluorophenyl)butanamido)-4-methylthiophen-3-yl)-7,10-diisopropyl-14-methyl-1,6,9,12-tetraoxo-2,5,8,11-tetraazapentadecan-13-yl)carbamate;
methyl 5-((3-((tert-butoxycarbonyl)amino)propyl)carbamoyl)-2-(2-(4-fluorophenyl)butanamido)-4-methylthiophene-3-carboxylate;
methyl 5-((3-aminopropyl)carbamoyl)-2-(2-(4-fluorophenyl)butanamido)-4-methylthiophene-3-carboxylate;
methyl 5-((2-(2-((tert-butoxycarbonyl)amino)ethoxy)ethyl)carbamoyl)-2-(2-(4-fluorophenyl)butanamido)-4-methylthiophene-3-carboxylate;
methyl 2-(2-(4-fluorophenyl)butanamido)-5-(((6S,9S)-6-isopropyl-2,2,9-trimethyl-4,7,10-trioxo-3-oxa-5,8,11-triazatridecan-13-yl)carbamoyl)-4-methylthiophene-3-carboxylate;
methyl 5-((2-((S)-2-((S)-2-amino-3-methylbutanamido)propanamido)ethyl)carbamoyl)-2-(2-(4-fluorophenyl)butanamido)-4-methylthiophene-3-carboxylate;
methyl 5-(((6S,9S,12S)-6,9-diisopropyl-2,2,12-trimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3,14-dioxa-5,8,11-triazahexadecan-16-yl)carbamoyl)-2-(2-(4-fluorophenyl)butanamido)-4-methylthiophene-3-carboxylate;
methyl 5-((3-((S)-2-((S)-2-amino-3-methylbutanamido)-3-methylbutanamido)propyl)carbamoyl)-2-(2-(4-fluorophenyl)butanamido)-4-methylthiophene-3-carboxylate;
methyl 5-((2-(2-((S)-2-((S)-2-amino-3-methylbutanamido)-3-methylbutanamido)ethoxy)ethyl)carbamoyl)-2-(2-(4-fluorophenyl)butanamido)-4-methylthiophene-3-carboxylate;
methyl 2-(2-(benzo[d][1,3]dioxol-5-yl)butanamido)-5-carbamoyl-4-methylthiophene-3-carboxylate;
2-(tert-butyl) 4-methyl 5-(2-(benzo[d][1,3]dioxol-5-yl)butanamido)-3-methylthiophene-2,4-dicarboxylate;
2-(tert-butyl) 4-methyl 5-(2-(3,5-difluorophenyl)butanamido)-3-methylthiophene-2,4-dicarboxylate:
methyl 5-carbamoyl-2-(2-(3,5-difluorophenyl)butanamido)-4-methylthiophene-3-carboxylate;
methyl 5-carbamoyl-2-(2-(4-cyanophenyl)butanamido)-4-methylthiophene-3-carboxylate;
methyl 5-((2-aminoethyl)carbamoyl)-2-(2-(benzo[d][1,3]dioxol-5-yl)butanamido)-4-methylthiophene-3-carboxylate;
methyl 2-(2-(benzo[d][1,3]dioxol-5-yl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(benzo[d][1,3]dioxol-5-yl)butanamido)-4-methylthiophene-3-carboxylate;
methyl 5-((2-aminoethyl)carbamoyl)-2-(2-(3,5-difluorophenyl)butanamido)-4-methylthiophene-3-carboxylate;
methyl 2-(2-(3,5-difluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(3,5-difluorophenyl)butanamido)-4-methylthiophene-3-carboxylate;
methyl 5-((2-aminoethyl)carbamoyl)-2-(2-(4-cyanophenyl)butanamido)-4-methylthiophene-3-carboxylate;
methyl 2-(2-(4-cyanophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(4-cyanophenyl)butanamido)-4-methylthiophene-3-carboxylate;
methyl 2-(2-(4-fluorophenyl)butanamido)-4-methyl-5-(((6S,9S,12S)-6,9,12-triisopropyl-2,2-dimethyl-4,7,10,13-tetraoxo-3,17-dioxa-5,8,11,14-tetraazanonadecan-19-yl)carbamoyl)thiophene-3-carboxylate;
methyl 5-(((8S,11S,14S)-14-amino-8,11-diisopropyl-15-methyl-7,10,13-trioxo-3-oxa-6,9,12-triazahexadecyl)carbamoyl)-2-(2-(4-fluorophenyl)butanamido)-4-methylthiophene-3-carboxylate;
2-(tert-butyl) 4-methyl 5-(2-(3,4-difluorophenyl)butanamido)-3-methylthiophene-2,4-dicarboxylate;
2-(tert-butyl) 4-methyl 3-methyl-5-(2-(m-tolyl)butanamido)thiophene-2,4-dicarboxylate;
2-(tert-butyl) 4-methyl 3-methyl-5-(2-(3-(trifluoromethyl)phenyl)butanamido)thiophene-2,4-dicarboxylate;
methyl 5-carbamoyl-2-(2-(3,4-difluorophenyl)butanamido)-4-methylthiophene-3-carboxylate;
methyl 5-carbamoyl-4-methyl-2-(2-(m-tolyl)butanamido)thiophene-3-carboxylate;
methyl 5-carbamoyl-4-methyl-2-(2-(3-(trifluoromethyl)phenyl)butanamido)thiophene-3-carboxylate;
methyl 5-((2-aminoethyl)carbamoyl)-2-(2-(3,4-difluorophenyl)butanamido)-4-methylthiophene-3-carboxylate;
methyl 2-(2-(3,4-difluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(3,4-difluorophenyl)butanamido)-4-methylthiophene-3-carboxylate;
methyl 5-((2-aminoethyl)carbamoyl)-4-methyl-2-(2-(m-tolyl)butanamido)thiophene-3-carboxylate;
methyl 4-methyl-2-(2-(m-tolyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(m-tolyl)butanamido)thiophene-3-carboxylate;
methyl 5-((2-aminoethyl)carbamoyl)-4-methyl-2-(2-(3-(trifluoromethyl)phenyl)butanamido)thiophene-3-carboxylate;
methyl 4-methyl-2-(2-(3-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(3-(trifluoromethyl)phenyl)butanamido)thiophene-3-carboxylate;
methyl 5-(((R)-1-(tert-butoxy)-3-methyl-1-oxobutan-2-yl)carbamoyl)-2-(2-(4-fluorophenyl)butanamido)-4-methylthiophene-3-carboxylate;
(5-(2-(4-fluorophenyl)butanamido)-4-(methoxycarbonyl)-3-methylthiophene-2-carbonyl)-D-valine;
2-(tert-butyl) 4-methyl 3-methyl-5-(2-(o-tolyl)butanamido)thiophene-2,4-dicarboxylate;
methyl 5-carbamoyl-4-methyl-2-(2-(o-tolyl)butanamido)thiophene-3-carboxylate;
methyl 5-((2-aminoethyl)carbamoyl)-4-methyl-2-(2-(o-tolyl)butanamido)thiophene-3-carboxylate;
methyl 4-methyl-2-(2-(o-tolyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(o-tolyl)butanamido)thiophene-3-carboxylate;
2-(tert-butyl) 4-methyl 3-methyl-5-(2-(2-(trifluoromethyl)phenyl)butanamido)thiophene-2,4-dicarboxylate;
methyl 5-carbamoyl-4-methyl-2-(2-(2-(trifluoromethyl)phenyl)butanamido)thiophene-3-carboxylate;
methyl 4-methyl-2-(2-(2-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(2-(trifluoromethyl)phenyl)butanamido)thiophene-3-carboxylate;
methyl 5-(((R)-1-(((R)-1-(tert-butoxy)-3-methyl-1-oxobutan-2-yl)amino)-3-methyl-1-oxobutan-2-yl)carbamoyl)-2-(2-(4-fluorophenyl)butanamido)-4-methylthiophene-3-carboxylate; and
(5-(2-(4-fluorophenyl)butanamido)-4-(methoxycarbonyl)-3-methylthiophene-2-carbonyl)-D-valyl-D-valine.

TABLE 1

| Cmpd | Structure |
|---|---|
| 1 | 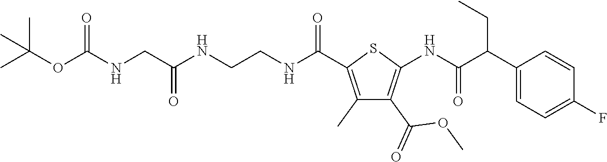 [SMILES: CCC(C(=O)Nc1sc(C(=O)NCCNC(=O)CNC(=O)OC(C)(C)C)c(C)c1C(=O)OC)c1ccc(F)cc1] |
| | methyl 5-((2-(2-((tert-butoxycarbonyl)amino)acetamido)ethyl)carbamoyl)-2-(2-(4- |
| | fluorophenyl)butanamido)-4-methylthiophene-3-carboxylate |
| 2 | 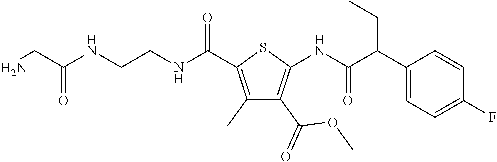 [SMILES: CCC(C(=O)Nc1sc(C(=O)NCCNC(=O)CN)c(C)c1C(=O)OC)c1ccc(F)cc1] |
| | methyl 5-((2-(2-aminoacetamido)ethyl)carbamoyl)-2-(2-(4- |
| | fluorophenyl)butanamido)-4-methylthiophene-3-carboxylate |
| 3 | 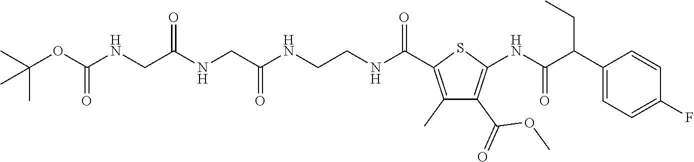 [SMILES: CCC(C(=O)Nc1sc(C(=O)NCCNC(=O)CNC(=O)CNC(=O)OC(C)(C)C)c(C)c1C(=O)OC)c1ccc(F)cc1] |
| | methyl 5-((2,2-dimethyl-4,7,10-trioxo-3-oxa-5,8,11-triazatridecan-13- |
| | yl)carbamoyl)-2-(2-(4-fluorophenyl)butanamido)-4-methylthiophene-3-carboxylate |
| 4 | 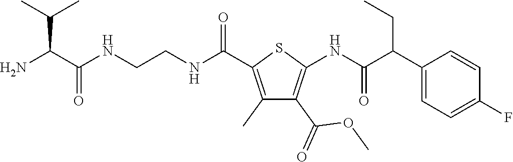 [SMILES: CCC(C(=O)Nc1sc(C(=O)NCCNC(=O)[C@@H](N)C(C)C)c(C)c1C(=O)OC)c1ccc(F)cc1] |
| | methyl 5-((2-((S)-2-amino-3-methylbutanamido)ethyl)carbamoyl)-2-(2-(4- |
| | fluorophenyl)butanamido)-4-methylthiophene-3-carboxylate |
| 5 | 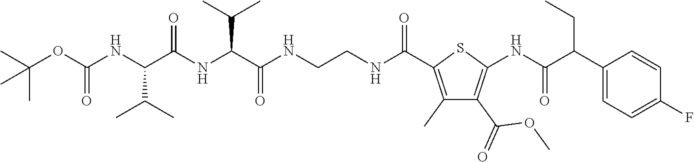 [SMILES: CCC(C(=O)Nc1sc(C(=O)NCCNC(=O)[C@@H](NC(=O)[C@@H](NC(=O)OC(C)(C)C)C(C)C)C(C)C)c(C)c1C(=O)OC)c1ccc(F)cc1] |
| | methyl 5-(((6S,9S)-6,9-diisopropyl-2,2-dimethyl-4,7,10-trioxo-3-oxa-5,8,11- |
| | triazatridecan-13-yl)carbamoyl)-2-(2-(4-fluorophenyl)butanamido)-4- |
| | methylthiophene-3-carboxylate |
| 6 | 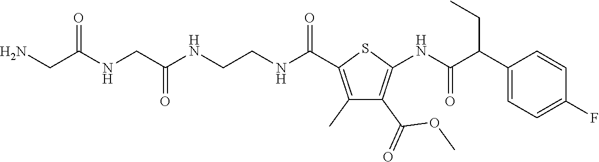 [SMILES: CCC(C(=O)Nc1sc(C(=O)NCCNC(=O)CNC(=O)CN)c(C)c1C(=O)OC)c1ccc(F)cc1] |
| | methyl 5-((2-(2-(2-aminoacetamido)acetamido)ethyl)carbamoyl)-2-(2-(4- |
| | fluorophenyl)butanamido)-4-methylthiophene-3-carboxylate |
| 7 | 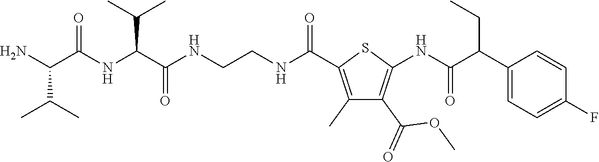 [SMILES: CCC(C(=O)Nc1sc(C(=O)NCCNC(=O)[C@@H](NC(=O)[C@@H](N)C(C)C)C(C)C)c(C)c1C(=O)OC)c1ccc(F)cc1] |
| | methyl 5-((2-((S)-2-((S)-2-amino-3-methylbutanamido)-3- |
| | methylbutanamido)ethyl)carbamoyl)-2-(2-(4-fluorophenyl)butanamido)-4- |
| | methylthiophene-3-carboxylate |
| 8 | 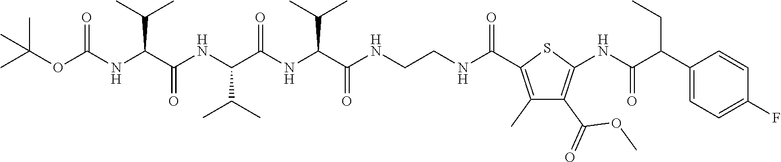 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc(F)cc1] |
| | methyl 2-(2-(4-fluorophenyl)butanamido)-4-methyl-5-(((6S,9S,12S)-6,9,12- |
| | triisopropyl-2,2-dimethyl-4,7,10,13-tetraoxo-3-oxa-5,8,11,14-tetraazahexadecan- |
| | 16-yl)carbamoyl)thiophene-3-carboxylate |
| 9 | 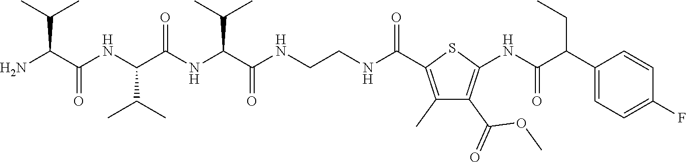 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccc(F)cc1] |
| | methyl 5-((2-((S)-2-((S)-2-((S)-2-amino-3-methylbutanamido)-3- |
| | methylbutanamido)-3-methylbutanamido)ethyl)carbamoyl)-2-(2-(4- |
| | fluorophenyl)butanamido)-4-methylthiophene-3-carboxylate |
| 10 | 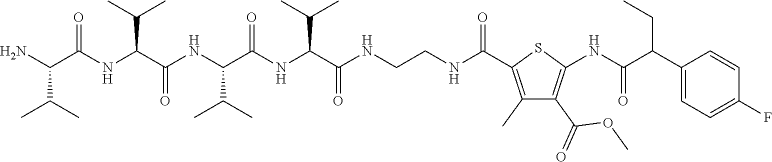 [SMILES: CCC(C(=O)Nc1sc(C(=O)NCCNC(=O)[C@@H](NC(=O)[C@@H](NC(=O)[C@@H](NC(=O)[C@@H](N)C(C)C)C(C)C)C(C)C)C(C)C)c(C)c1C(=O)OC)c1ccc(F)cc1] |
| | methyl 5-(((5S,8S,11S,14S)-14-amino-5,8,11-triisopropyl-15-methyl-4,7,10,13- |
| | tetraoxo-3,6,9,12-tetraazahexadecyl)carbamoyl)-2-(2-(4-fluorophenyl)butanamido)- |
| | 4-methylthiophene-3-carboxylate |
| 11 | 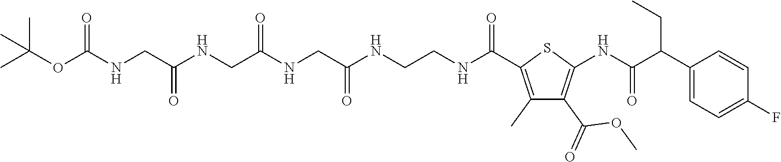 [SMILES: CCC(C(=O)Nc1sc(C(=O)NCCNC(=O)CNC(=O)CNC(=O)CNC(=O)OC(C)(C)C)c(C)c1C(=O)OC)c1ccc(F)cc1] |
| | methyl 5-((2,2-dimethyl-4,7,10,13-tetraoxo-3-oxa-5,8,11,14-tetraazahexadecan-16- |
| | yl)carbamoyl)-2-(2-(4-fluorophenyl)butanamido)-4-methylthiophene-3-carboxylate |
| 12 | 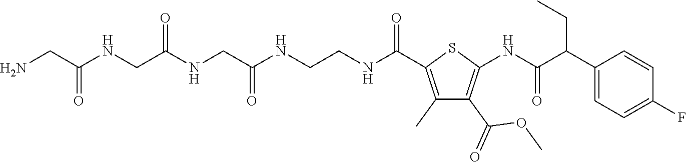 [SMILES: CCC(C(=O)Nc1sc(C(=O)NCCNC(=O)CNC(=O)CNC(=O)CN)c(C)c1C(=O)OC)c1ccc(F)cc1] |
| | methyl 5-((2-(2-(2-(2-aminoacetamido)acetamido)acetamido)ethyl)carbamoyl)-2- |
| | (2-(4-fluorophenyl)butanamido)-4-methylthiophene-3-carboxylate |
| 13 | 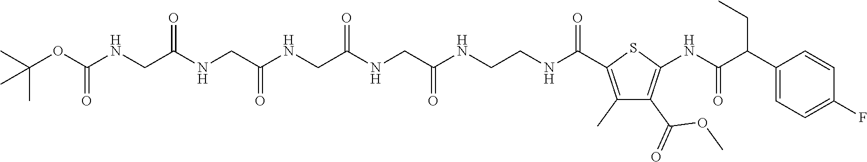 [SMILES: CCC(C(=O)Nc1sc(C(=O)NCCNC(=O)CNC(=O)CNC(=O)CNC(=O)CNC(=O)OC(C)(C)C)c(C)c1C(=O)OC)c1ccc(F)cc1] |
| | methyl 5-((2,2-dimethyl-4,7,10,13,16-pentaoxo-3-oxa-5,8,11,14,17- |
| | pentaazanonadecan-19-yl)carbamoyl)-2-(2-(4-fluorophenyl)butanamido)-4- |
| | methylthiophene-3-carboxylate |
| 14 | 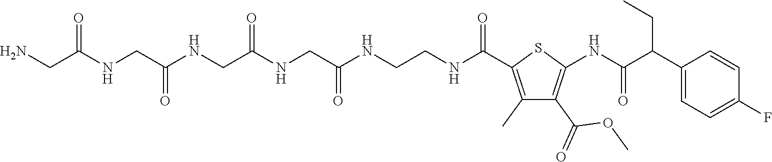 [SMILES: CCC(C(=O)Nc1sc(C(=O)NCCNC(=O)CNC(=O)CNC(=O)CNC(=O)CN)c(C)c1C(=O)OC)c1ccc(F)cc1] |
| | methyl 5-((14-amino-4,7,10,13-tetraoxo-3,6,9,12-tetraazatetradecyl)carbamoy1)-2- |
| | (2-(4-fluorophenyl)butanamido)-4-methylthiophene-3-carboxylate |
| 15 | 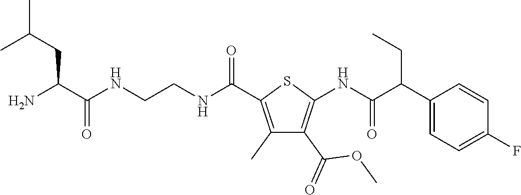 [SMILES: CCC(C(=O)Nc1sc(C(=O)NCCNC(=O)[C@@H](N)CC(C)C)c(C)c1C(=O)OC)c1ccc(F)cc1] |
| | methyl 5-((2-((S)-2-amino-4-methylpentanamido)ethyl)carbamoyl)-2-(2-(4- |
| | fluorophenyl)butanamido)-4-methylthiophene-3-carboxylate |
| 16 | 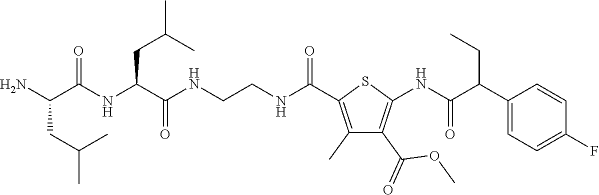 [SMILES: CCC(C(=O)Nc1sc(C(=O)NCCNC(=O)[C@H](CC(C)C)NC(=O)[C@@H](N)CC(C)C)c(C)c1C(=O)OC)c1ccc(F)cc1] |
| | methyl 5-((2-((S)-2-((S)-2-amino-4-methylpentanamido)-4- |
| | methylpentanamido)ethyl)carbamoyl)-2-(2-(4-fluorophenyl)butanamido)-4- |
| | methylthiophene-3-carboxylate |
| 17 | 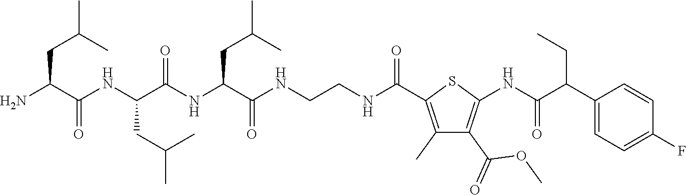 [SMILES: CCC(C(=O)Nc1sc(C(=O)NCCNC(=O)[C@H](CC(C)C)NC(=O)[C@H](CC(C)C)NC(=O)[C@@H](N)CC(C)C)c(C)c1C(=O)OC)c1ccc(F)cc1] |
| | methyl 5-((2-((S)-2-((S)-2-((S)-2-amino-4-methylpentanamido)-4- |
| | methylpentanamido)-4-methylpentanamido)ethyl)carbamoyl)-2-(2-(4- |
| | fluorophenyl)butanamido)-4-methylthiophene-3-carboxylate |
| 18 | 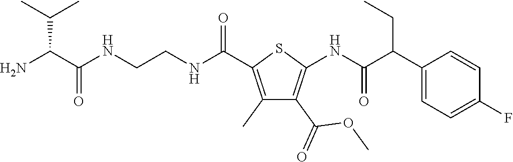 [SMILES: CCC(C(=O)Nc1sc(C(=O)NCCNC(=O)[C@H](N)C(C)C)c(C)c1C(=O)OC)c1ccc(F)cc1] |
| | methyl 5-((2-((R)-2-amino-3-methylbutanamido)ethyl)carbamoyl)-2-(2-(4- |
| | fluorophenyl)butanamido)-4-methylthiophene-3-carboxylate |
| 19 | 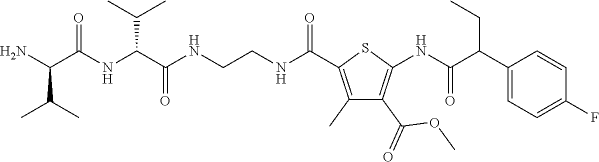 [SMILES: CCC(C(=O)Nc1sc(C(=O)NCCNC(=O)[C@H](NC(=O)[C@H](N)C(C)C)C(C)C)c(C)c1C(=O)OC)c1ccc(F)cc1] |
| | methyl 5-((2-((R)-2-((R)-2-amino-3-methylbutanamido)-3- |
| | methylbutanamido)ethyl)carbamoyl)-2-(2-(4-fluorophenyl)butanamido)-4- |
| | methylthiophene-3-carboxylate |
| 20 | 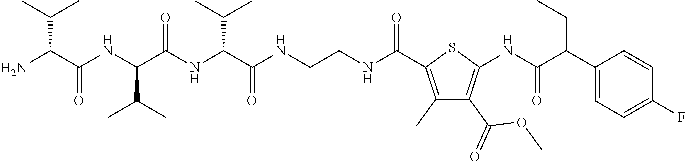 [SMILES: CCC(C(=O)Nc1sc(C(=O)NCCNC(=O)[C@H](NC(=O)[C@H](NC(=O)[C@H](N)C(C)C)C(C)C)C(C)C)c(C)c1C(=O)OC)c1ccc(F)cc1] |
| | methyl 5-((2-((R)-2-((R)-2-((R)-2-amino-3-methylbutanamido)-3- |
| | methylbutanamido)-3-methylbutanamido)ethyl)carbamoyl)-2-(2-(4- |
| | fluorophenyl)butanamido)-4-methylthiophene-3-carboxylate |
| 21 | 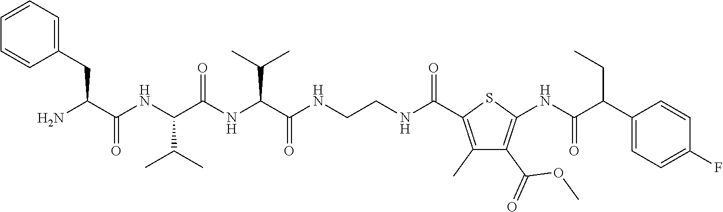 [SMILES: CCC(C(=O)Nc1sc(C(=O)NCCNC(=O)[C@@H](NC(=O)[C@@H](NC(=O)[C@@H](N)Cc2ccccc2)C(C)C)C(C)C)c(C)c1C(=O)OC)c1ccc(F)cc1] |
| | methyl 5-((2-((S)-2-((S)-2-((S)-2-amino-3-phenylpropanamido)-3- |
| | methylbutanamido)-3-methylbutanamido)ethyl)carbamoy1)-2-(2-(4- |
| | fluorophenyl)butanamido)-4-methylthiophene-3-carboxylate |
| 22 | 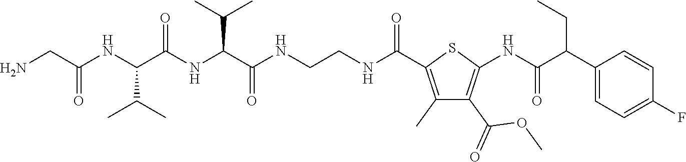 [SMILES: CCC(C(=O)Nc1sc(C(=O)NCCNC(=O)[C@@H](NC(=O)[C@@H](NC(=O)CN)C(C)C)C(C)C)c(C)c1C(=O)OC)c1ccc(F)cc1] |
| | methyl 5-((2-((S)-2-((S)-2-(2-aminoacetamido)-3-methylbutanamido)-3- |
| | methylbutanamido)ethyl)carbamoyl)-2-(2-(4-fluorophenyl)butanamido)-4- |
| | methylthiophene-3-carboxylate |
| 23 | 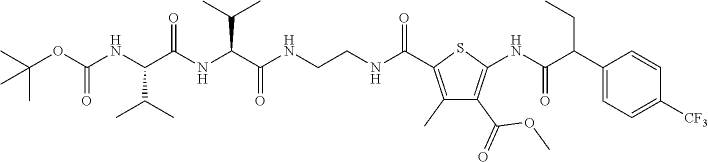 [SMILES: CCC(C(=O)Nc1sc(C(=O)NCCNC(=O)[C@@H](NC(=O)[C@@H](NC(=O)OC(C)(C)C)C(C)C)C(C)C)c(C)c1C(=O)OC)c1ccc(C(F)(F)F)cc1] |
| | methyl 5-(((6S,9S)-6,9-diisopropyl-2,2-dimethyl-4,7,10-trioxo-3-oxa-5,8,11- |
| | triazatridecan-13-yl)carbamoyl)-4-methyl-2-(2-(4- |
| | (trifluoromethyl)phenyl)butanamido)thiophene-3-carboxylate |
| 24 | 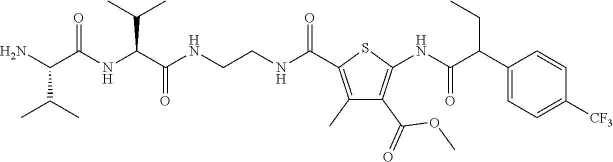 [SMILES: CCC(C(=O)Nc1sc(C(=O)NCCNC(=O)[C@@H](NC(=O)[C@@H](N)C(C)C)C(C)C)c(C)c1C(=O)OC)c1ccc(C(F)(F)F)cc1] |
| | methyl 5-((2-((S)-2-((S)-2-amino-3-methylbutanamido)-3- |
| | methylbutanamido)ethyl)carbamoyl)-4-methyl-2-(2-(4- |
| | (trifluoromethyl)phenyl)butanamido)thiophene-3-carboxylate |
| 25 | 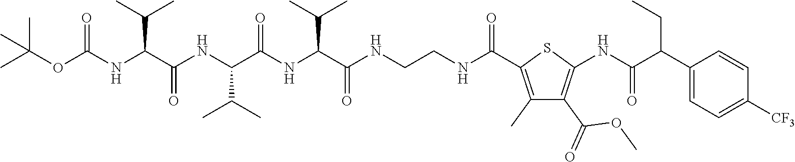 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc(C(F)(F)F)cc1] |
| | methyl 4-methyl-2-(2-(4-(trifluoromethyl)phenyl)butanamido)-5-(((6S,9S,12S)- |
| | 6,9,12-triisopropyl-2,2-dimethyl-4,7,10,13-tetraoxo-3-oxa-5,8,11,14- |
| | tetraazahexadecan-16-yl)carbamoyl)thiophene-3-carboxylate |
| 26 | 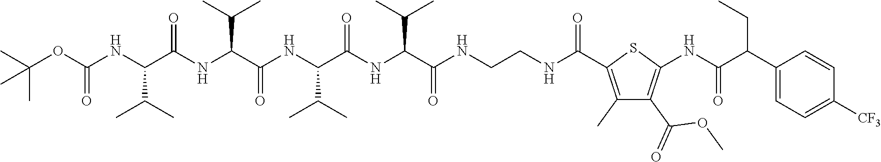 [SMILES: CCC(C(=O)Nc1sc(C(=O)NCCNC(=O)[C@@H](NC(=O)[C@@H](NC(=O)[C@@H](NC(=O)[C@@H](NC(=O)OC(C)(C)C)C(C)C)C(C)C)C(C)C)C(C)C)c(C)c1C(=O)OC)c1ccc(C(F)(F)F)cc1] |
| | methyl 4-methyl-5-(((6S,9S,12S,15S)-6,9,12,15-tetraisopropyl-2,2-dimethyl- |
| | 4,7,10,13,16-pentaoxo-3-oxa-5,8,11,14,17-pentaazanonadecan-19-yl)carbamoy1)-2- |
| | (2-(4-(trifluoromethyl)phenyl)butanamido)thiophene-3-carboxylate |
| 27 | 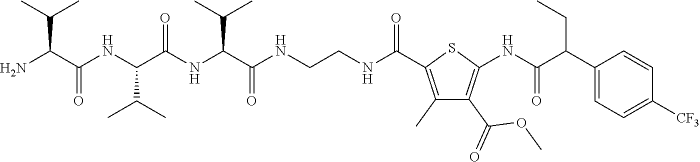 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccc(C(F)(F)F)cc1] |
| | methyl 5-((2-((S)-2-((S)-2-((S)-2-amino-3-methylbutanamido)-3- |
| | methylbutanamido)-3-methylbutanamido)ethyl)carbamoyl)-4-methyl-2-(2-(4- |
| | (trifluoromethyl)phenyl)butanamido)thiophene-3-carboxylate |
| 28 | 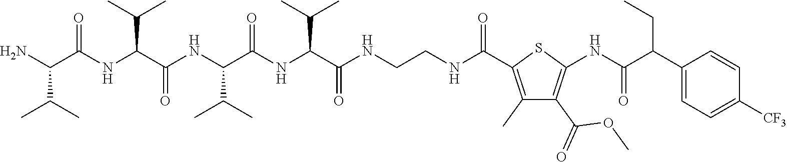 [SMILES: CCC(C(=O)Nc1sc(C(=O)NCCNC(=O)[C@@H](NC(=O)[C@@H](NC(=O)[C@@H](NC(=O)[C@@H](N)C(C)C)C(C)C)C(C)C)C(C)C)c(C)c1C(=O)OC)c1ccc(C(F)(F)F)cc1] |
| | methyl 5-(((5S,8S,11S,14S)-14-amino-5,8,11-triisopropyl-15-methyl-4,7,10,13- |
| | tetraoxo-3,6,9,12-tetraazahexadecyl)carbamoyl)-4-methyl-2-(2-(4- |
| | (trifluoromethyl)phenyl)butanamido)thiophene-3-carboxylate |
| 29 | 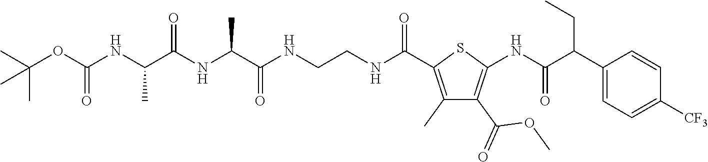 [SMILES: CCC(C(=O)Nc1sc(C(=O)NCCNC(=O)[C@H](C)NC(=O)[C@H](C)NC(=O)OC(C)(C)C)c(C)c1C(=O)OC)c1ccc(C(F)(F)F)cc1] |
| | methyl 4-methyl-5-(((6S,9S)-2,2,6,9-tetramethyl-4,7,10-trioxo-3-oxa-5,8,11- |
| | triazatridecan-13-yl)carbamoyl)-2-(2-(4- |
| | (trifluoromethyl)phenyl)butanamido)thiophene-3-carboxylate |
| 30 | 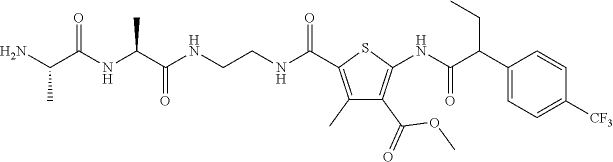 [SMILES: CCC(C(=O)Nc1sc(C(=O)NCCNC(=O)[C@H](C)NC(=O)[C@H](C)N)c(C)c1C(=O)OC)c1ccc(C(F)(F)F)cc1] |
| | methyl 5-((2-((S)-2-((S)-2-aminopropanamido)propanamido)ethyl)carbamoy1)-4- |
| | methyl-2-(2-(4-(trifluoromethyl)phenyl)butanamido)thiophene-3-carboxylate |
| 31 | 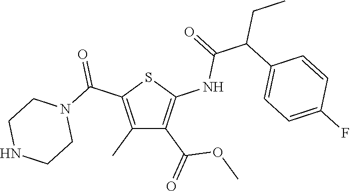 [SMILES: CCC(C(=O)Nc1sc(C(=O)N2CCNCC2)c(C)c1C(=O)OC)c1ccc(F)cc1] |
| | methyl 2-(2-(4-fluorophenyl)butanamido)-4-methyl-5-(piperazine-1- |
| | carbonyl)thiophene-3-carboxylate |
| 32 | 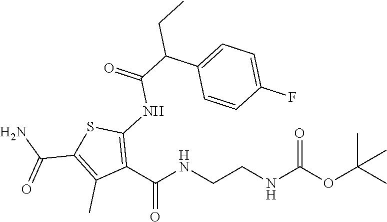 [SMILES: CCC(C(=O)Nc1sc(C(N)=O)c(C)c1C(=O)NCCNC(=O)OC(C)(C)C)c1ccc(F)cc1] |
| | tert-butyl (2-(5-carbamoyl-2-(2-(4-fluorophenyl)butanamido)-4-methylthiophene-3- |
| | carboxamido)ethyl)carbamate |
| 33 | 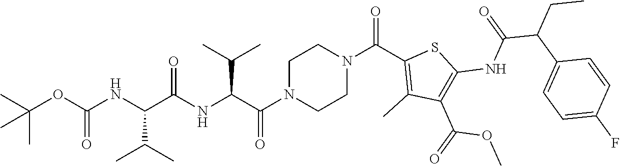 [SMILES: CCC(C(=O)Nc1sc(C(=O)N2CCN(C(=O)[C@@H](NC(=O)[C@@H](NC(=O)OC(C)(C)C)C(C)C)C(C)C)CC2)c(C)c1C(=O)OC)c1ccc(F)cc1] |
| | methyl 5-(4-((tert-butoxycarbonyl)-L-valy1-L-valy1)piperazine-1-carbony1)-2-(2-(4- |
| | fluorophenyl)butanamido)-4-methylthiophene-3-carboxylate |
| 34 | 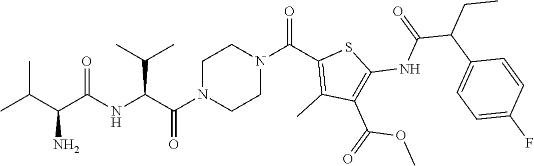 [SMILES: CCC(C(=O)Nc1sc(C(=O)N2CCN(C(=O)[C@@H](NC(=O)[C@@H](N)C(C)C)C(C)C)CC2)c(C)c1C(=O)OC)c1ccc(F)cc1] |
| | methyl 5-(4-(L-valyl-L-valyl)piperazine-1-carbonyl)-2-(2-(4- |
| | fluorophenyl)butanamido)-4-methylthiophene-3-carboxylate |
| 35 | 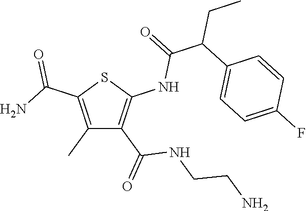 [SMILES: CCC(C(=O)Nc1sc(C(N)=O)c(C)c1C(=O)NCCN)c1ccc(F)cc1] |
| | N4-(2-aminoethyl)-5-(2-(4-fluorophenyl)butanamido)-3-methylthiophene-2,4- |
| | dicarboxamide |
| 36 | 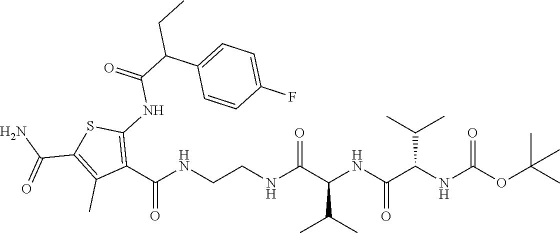 [SMILES: CCC(C(=O)Nc1sc(C(N)=O)c(C)c1C(=O)NCCNC(=O)[C@@H](NC(=O)[C@@H](NC(=O)OC(C)(C)C)C(C)C)C(C)C)c1ccc(F)cc1] |
| | tert-butyl ((2S)-1-(((2S)-1-((2-(5-carbamoy1-2-(2-(4-fluorophenyl)butanamido)-4- |
| | methylthiophene-3-carboxamido)ethyl)amino)-3-methyl-1-oxobutan-2-yl)amino)-3- |
| | methyl-1-oxobutan-2-yl)carbamate |
| 37 | 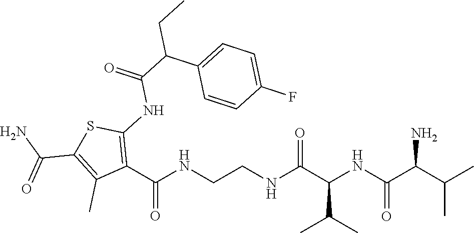 [SMILES: CCC(C(=O)Nc1sc(C(N)=O)c(C)c1C(=O)NCCNC(=O)[C@@H](NC(=O)[C@@H](N)C(C)C)C(C)C)c1ccc(F)cc1] |
| | N4-(2-((S)-2-((S)-2-amino-3-methylbutanamido)-3-methylbutanamido)ethyl)-5-(2- |
| | (4-fluorophenyl)butanamido)-3-methylthiophene-2,4-dicarboxamide |
| 38 | 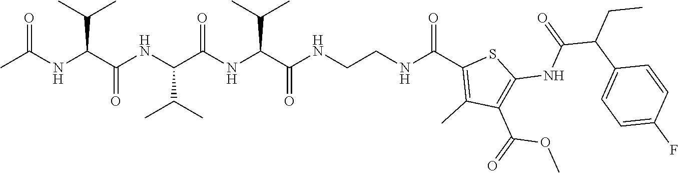 [SMILES: CCC(C(=O)Nc1sc(C(=O)NCCNC(=O)[C@@H](NC(=O)[C@@H](NC(=O)[C@@H](NC(C)=O)C(C)C)C(C)C)C(C)C)c(C)c1C(=O)OC)c1ccc(F)cc1] |
| | methyl 2-(2-(4-fluorophenyl)butanamido)-4-methyl-5-(((5S,8S,11S)-5,8,11- |
| | triisopropyl-4,7,10,13-tetraoxo-3,6,9,12-tetraazatetradecyl)carbamoyl)thiophene-3- |
| | carboxylate |
| 39 | 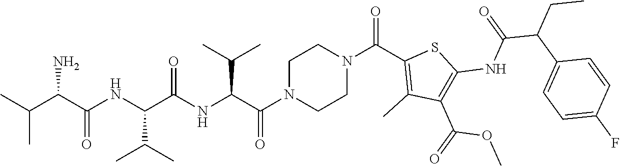 [SMILES: CCC(C(=O)Nc1sc(C(=O)N2CCN(C(=O)[C@@H](NC(=O)[C@@H](NC(=O)[C@@H](N)C(C)C)C(C)C)C(C)C)CC2)c(C)c1C(=O)OC)c1ccc(F)cc1] |
| | methyl 5-(4-(L-valyl-L-valyl-L-valyl)piperazine-1-carbonyl)-2-(2-(4- |
| | fluorophenyl)butanamido)-4-methylthiophene-3-carboxylate |
| 40 | 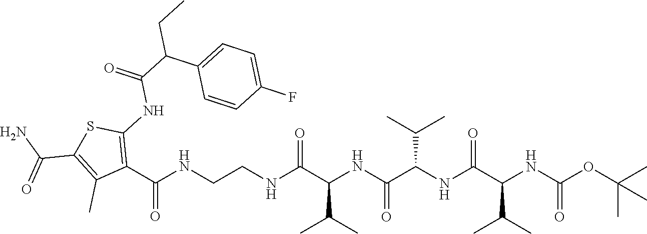 [SMILES: CCC(C(=O)Nc1sc(C(N)=O)c(C)c1C(=O)NCCNC(=O)[C@@H](NC(=O)[C@@H](NC(=O)[C@@H](NC(=O)OC(C)(C)C)C(C)C)C(C)C)C(C)C)c1ccc(F)cc1] |
| | tert-butyl ((7S,10S,13S)-1-(5-carbamoyl-2-(2-(4-fluorophenyl)butanamido)-4- |
| | methylthiophen-3-yl)-7,10-diisopropyl-14-methyl-1,6,9,12-tetraoxo-2,5,8,11- |
| | tetraazapentadecan-13-yl)carbamate |
| 41 | 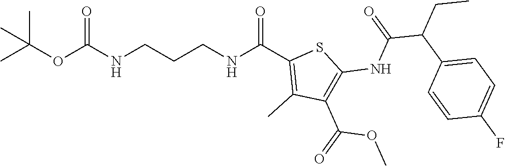 [SMILES: CCC(C(=O)Nc1sc(C(=O)NCCCNC(=O)OC(C)(C)C)c(C)c1C(=O)OC)c1ccc(F)cc1] |
| | methyl 5-((3-((tert-butoxycarbonyl)amino)propyl)carbamoyl)-2-(2-(4- |
| | fluorophenyl)butanamido)-4-methylthiophene-3-carboxylate |
| 42 | 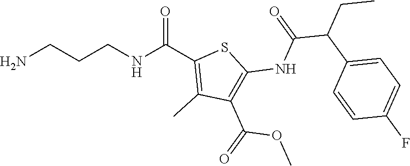 [SMILES: CCC(C(=O)Nc1sc(C(=O)NCCCN)c(C)c1C(=O)OC)c1ccc(F)cc1] |
| | methyl 5-((3-aminopropyl)carbamoy1)-2-(2-(4-fluorophenyl)butanamido)-4- |
| | methylthiophene-3-carboxylate |
| 43 | 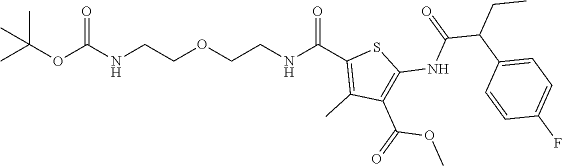 [SMILES: CCC(C(=O)Nc1sc(C(=O)NCCOCCNC(=O)OC(C)(C)C)c(C)c1C(=O)OC)c1ccc(F)cc1] |
| | methyl 5-((2-(2-((tert-butoxycarbonyl)amino)ethoxy)ethyl)carbamoyl)-2-(2-(4- |
| | fluorophenyl)butanamido)-4-methylthiophene-3-carboxylate |
| 44 | 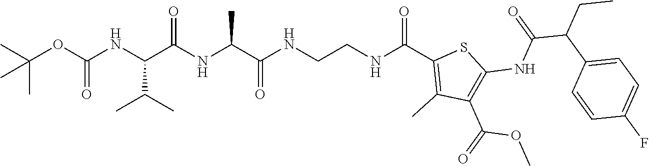 [SMILES: CCC(C(=O)Nc1sc(C(=O)NCCNC(=O)[C@H](C)NC(=O)[C@@H](NC(=O)OC(C)(C)C)C(C)C)c(C)c1C(=O)OC)c1ccc(F)cc1] |
| | methyl 2-(2-(4-fluorophenyl)butanamido)-5-(((6S,9S)-6-isopropyl-2,2,9-trimethyl- |
| | 4,7,10-trioxo-3-oxa-5,8,11-triazatridecan-13-yl)carbamoyl)-4-methylthiophene-3- |
| | carboxylate |
| 45 | 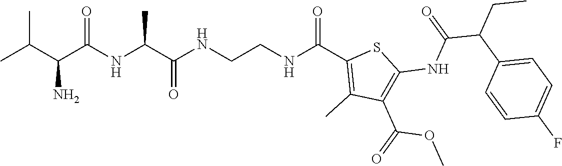 [SMILES: CCC(C(=O)Nc1sc(C(=O)NCCNC(=O)[C@H](C)NC(=O)[C@@H](N)C(C)C)c(C)c1C(=O)OC)c1ccc(F)cc1] |
| | methyl 5-((2-((S)-2-((S)-2-amino-3- |
| | methylbutanamido)propanamido)ethyl)carbamoyl)-2-(2-(4- |
| | fluorophenyl)butanamido)-4-methylthiophene-3-carboxylate |
| 46 | 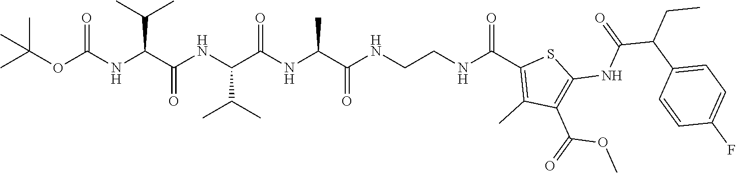 [SMILES: CCC(C(=O)Nc1sc(C(=O)NCCNC(=O)[C@H](C)NC(=O)[C@@H](NC(=O)[C@@H](NC(=O)OC(C)(C)C)C(C)C)C(C)C)c(C)c1C(=O)OC)c1ccc(F)cc1] |
| | methyl 5-(((6S,9S,12S)-6,9-diisopropyl-2,2,12-trimethyl-4,7,10,13-tetraoxo-3-oxa- |
| | 5,8,11,14-tetraazahexadecan-16-yl)carbamoyl)-2-(2-(4-fluorophenyl)butanamido)- |
| | 4-methylthiophene-3-carboxylate |
| 47 | 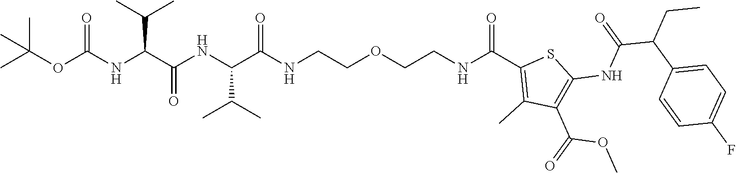 [SMILES: CCC(C(=O)Nc1sc(C(=O)NCCOCCNC(=O)[C@@H](NC(=O)[C@@H](NC(=O)OC(C)(C)C)C(C)C)C(C)C)c(C)c1C(=O)OC)c1ccc(F)cc1] |
| | methyl 5-(((6S,9S)-6,9-diisopropyl-2,2-dimethyl-4,7,10-trioxo-3,14-dioxa-5,8,11- |
| | triazahexadecan-16-yl)carbamoyl)-2-(2-(4-fluorophenyl)butanamido)-4- |
| | methylthiophene-3-carboxylate |
| 48 | 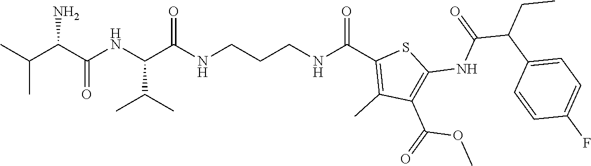 [SMILES: CCC(C(=O)Nc1sc(C(=O)NCCCNC(=O)[C@@H](NC(=O)[C@@H](N)C(C)C)C(C)C)c(C)c1C(=O)OC)c1ccc(F)cc1] |
| | methyl 5-((3-((S)-2-((S)-2-amino-3-methylbutanamido)-3- |
| | methylbutanamido)propyl)carbamoyl)-2-(2-(4-fluorophenyl)butanamido)-4- |
| | methylthiophene-3-carboxylate |
| 49 | 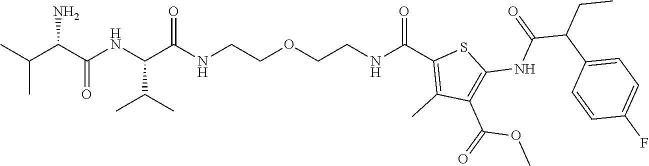 [SMILES: CCC(C(=O)Nc1sc(C(=O)NCCOCCNC(=O)[C@@H](NC(=O)[C@@H](N)C(C)C)C(C)C)c(C)c1C(=O)OC)c1ccc(F)cc1] |
| | methyl 5-((2-(2-((S)-2-((S)-2-amino-3-methylbutanamido)-3- |
| | methylbutanamido)ethoxy)ethyl)carbamoyl)-2-(2-(4-fluorophenyl)butanamido)-4- |
| | methylthiophene-3-carboxylate |
| 50 | 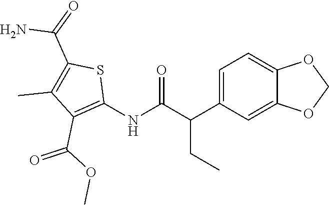 [SMILES: CCC(C(=O)Nc1sc(C(N)=O)c(C)c1C(=O)OC)c1ccc2c(c1)OCO2] |
| | methyl 2-(2-(benzo[d][1,3]dioxol-5-yl)butanamido)-5-carbamoyl-4- |
| | methylthiophene-3-carboxylate |
| 51 | 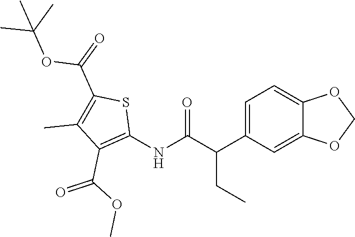 [SMILES: CCC(C(=O)Nc1sc(C(=O)OC(C)(C)C)c(C)c1C(=O)OC)c1ccc2c(c1)OCO2] |
| | 2-(tert-butyl) 4-methyl 5-(2-(benzo[d][1,3]dioxol-5-yl)butanamido)-3- |
| | methylthiophene-2,4-dicarboxylate |
| 52 | 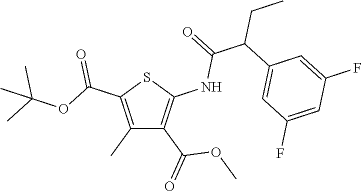 [SMILES: CCC(C(=O)Nc1sc(C(=O)OC(C)(C)C)c(C)c1C(=O)OC)c1cc(F)cc(F)c1] |
| | 2-(tert-butyl) 4-methyl 5-(2-(3,5-difluorophenyl)butanamido)-3-methylthiophene- |
| | 2,4-dicarboxylate |
| 53 | 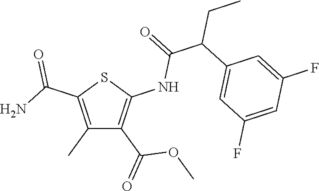 [SMILES: CCC(C(=O)Nc1sc(C(N)=O)c(C)c1C(=O)OC)c1cc(F)cc(F)c1] |
| | methyl 5-carbamoyl-2-(2-(3,5-difluorophenyl)butanamido)-4-methylthiophene-3- |
| | carboxylate |
| 54 | 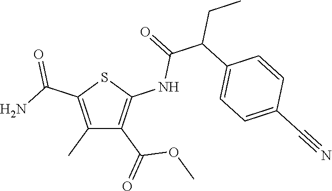 [SMILES: CCC(C(=O)Nc1sc(C(N)=O)c(C)c1C(=O)OC)c1ccc(C#N)cc1] |
| | methyl 5-carbamoyl-2-(2-(4-cyanophenyl)butanamido)-4-methylthiophene-3- |
| | carboxylate |
| 55 | 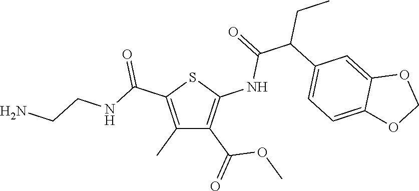 [SMILES: CCC(C(=O)Nc1sc(C(=O)NCCN)c(C)c1C(=O)OC)c1ccc2c(c1)OCO2] |
| | methyl 5-((2-aminoethyl)carbamoyl)-2-(2-(benzo[d][1,3]dioxol-5-yl)butanamido)- |
| | 4-methylthiophene-3-carboxylate |
| 56 | 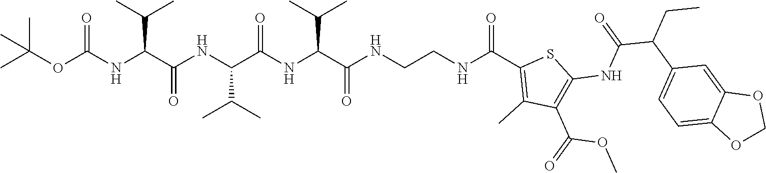 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc2c(c1)OCO2] |
| | methyl 2-(2-(benzo[d][1,3]dioxol-5-yl)butanamido)-4-methyl-5-(((6S,9S,12S)- |
| | 6,9,12-triisopropyl-2,2-dimethyl-4,7,10,13-tetraoxo-3-oxa-5,8,11,14- |
| | tetraazahexadecan-16-yl)carbamoyl)thiophene-3-carboxylate |
| 57 | 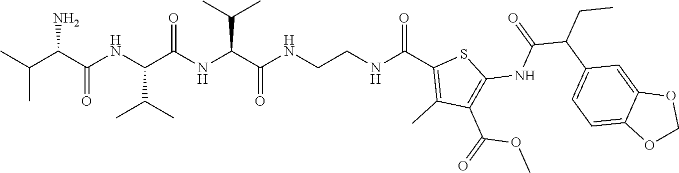 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccc2c(c1)OCO2] |
| | methyl 5-((2-((S)-2-((S)-2-((S)-2-amino-3-methylbutanamido)-3- |
| | methylbutanamido)-3-methylbutanamido)ethyl)carbamoyl)-2-(2- |
| | (benzo[d][1,3]dioxol-5-yl)butanamido)-4-methylthiophene-3-carboxylate |
| 58 | 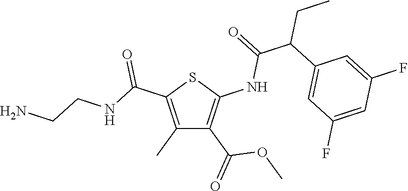 [SMILES: CCC(C(=O)Nc1sc(C(=O)NCCN)c(C)c1C(=O)OC)c1cc(F)cc(F)c1] |
| | methyl 5-((2-aminoethyl)carbamoyl)-2-(2-(3,5-difluorophenyl)butanamido)-4- |
| | methylthiophene-3-carboxylate |
| 59 | 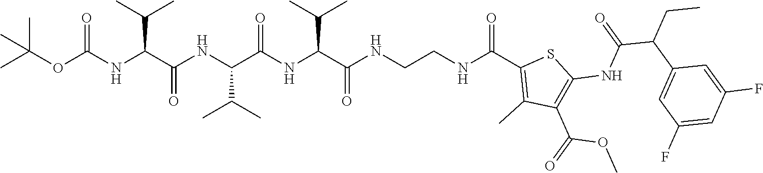 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1cc(F)cc(F)c1] |
| | methyl 2-(2-(3,5-difluorophenyl)butanamido)-4-methyl-5-(((6S,9S,12S)-6,9,12- |
| | triisopropyl-2,2-dimethyl-4,7,10,13-tetraoxo-3-oxa-5,8,11,14-tetraazahexadecan- |
| | 16-yl)carbamoyl)thiophene-3-carboxylate |
| 60 | 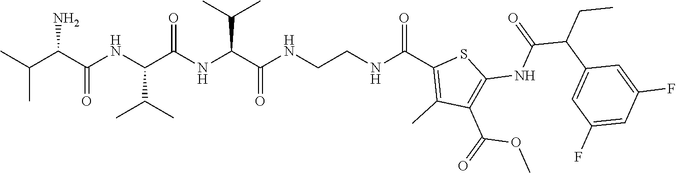 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1cc(F)cc(F)c1] |
| | methyl 5-((2-((S)-2-((S)-2-((S)-2-amino-3-methylbutanamido)-3- |
| | methylbutanamido)-3-methylbutanamido)ethyl)carbamoyl)-2-(2-(3,5- |
| | difluorophenyl)butanamido)-4-methylthiophene-3-carboxylate |
| 61 | 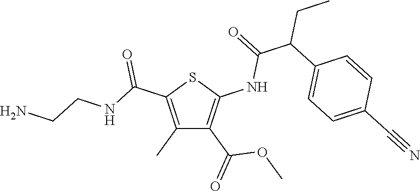 [SMILES: CCC(C(=O)Nc1sc(C(=O)NCCN)c(C)c1C(=O)OC)c1ccc(C#N)cc1] |
| | methyl 5-((2-aminoethyl)carbamoyl)-2-(2-(4-cyanophenyl)butanamido)-4- |
| | methylthiophene-3-carboxylate |
| 62 | 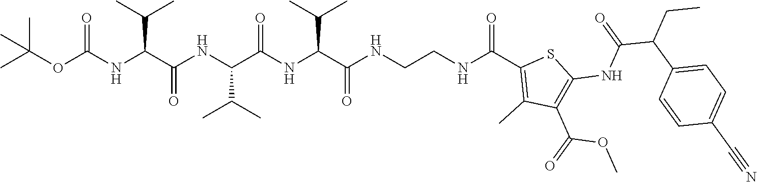 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc(C#N)cc1] |
| | methyl 2-(2-(4-cyanophenyl)butanamido)-4-methyl-5-(((6S,9S,12S)-6,9,12- |
| | triisopropyl-2,2-dimethyl-4,7,10,13-tetraoxo-3-oxa-5,8,11,14-tetraazahexadecan- |
| | 16-yl)carbamoyl)thiophene-3-carboxylate |
| 63 | 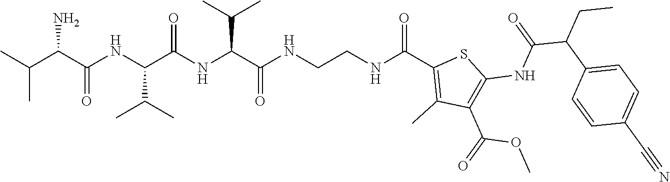 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccc(C#N)cc1] |
| | methyl 5-((2-((S)-2-((S)-2-((S)-2-amino-3-methylbutanamido)-3- |
| | methylbutanamido)-3-methylbutanamido)ethyl)carbamoyl)-2-(2-(4- |
| | cyanophenyl)butanamido)-4-methylthiophene-3-carboxylate |
| 64 | 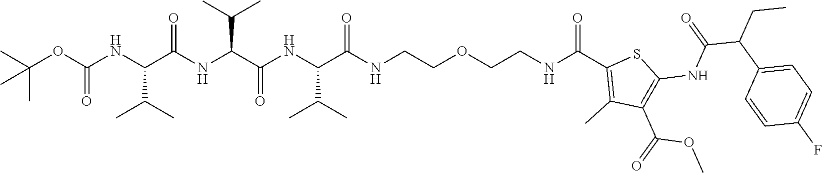 [SMILES: CCC(C(=O)Nc1sc(C(=O)NCCOCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc(F)cc1] |
| | methyl 2-(2-(4-fluorophenyl)butanamido)-4-methyl-5-(((6S,9S,12S)-6,9,12- |
| | triisopropyl-2,2-dimethyl-4,7,10,13-tetraoxo-3,17-dioxa-5,8,11,14- |
| | tetraazanonadecan-19-yl)carbamoyl)thiophene-3-carboxylate |
| 65 | 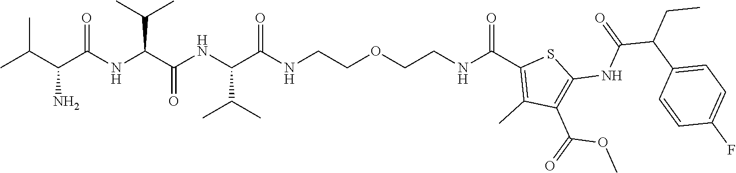 [SMILES: CCC(C(=O)Nc1sc(C(=O)NCCOCCNC(=O)[C@@H](NC(=O)[C@@H](NC(=O)[C@H](N)C(C)C)C(C)C)C(C)C)c(C)c1C(=O)OC)c1ccc(F)cc1] |
| | methyl 5-(((8S,11S,14S)-14-amino-8,11-diisopropyl-15-methyl-7,10,13-trioxo-3- |
| | oxa-6,9,12-triazahexadecyl)carbamoyl)-2-(2-(4-fluorophenyl)butanamido)-4- |
| | methylthiophene-3-carboxylate |
| 66 | 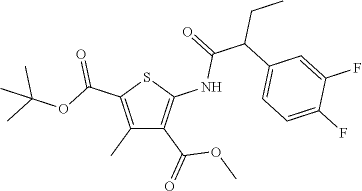 [SMILES: CCC(C(=O)Nc1sc(C(=O)OC(C)(C)C)c(C)c1C(=O)OC)c1ccc(F)c(F)c1] |
| | 2-(tert-butyl) 4-methyl 5-(2-(3,4-difluorophenyl)butanamido)-3-methylthiophene- |
| | 2,4-dicarboxylate |
| 67 | 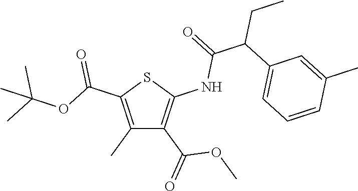 [SMILES: CCC(C(=O)Nc1sc(C(=O)OC(C)(C)C)c(C)c1C(=O)OC)c1cccc(C)c1] |
| | 2-(tert-butyl) 4-methyl 3-methyl-5-(2-(m-tolyl)butanamido)thiophene-2,4- |
| | dicarboxylate |
| 68 | 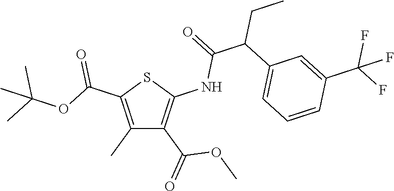 [SMILES: CCC(C(=O)Nc1sc(C(=O)OC(C)(C)C)c(C)c1C(=O)OC)c1cccc(C(F)(F)F)c1] |
| | 2-(tert-butyl) 4-methyl 3-methyl-5-(2-(3- |
| | (trifluoromethyl)phenyl)butanamido)thiophene-2,4-dicarboxylate |
| 69 | 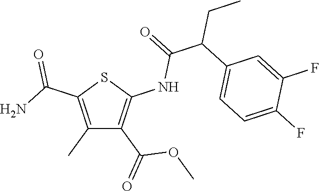 [SMILES: CCC(C(=O)Nc1sc(C(N)=O)c(C)c1C(=O)OC)c1ccc(F)c(F)c1] |
| | methyl 5-carbamoy1-2-(2-(3,4-difluorophenyl)butanamido)-4-methylthiophene-3- |
| | carboxylate |
| 70 | 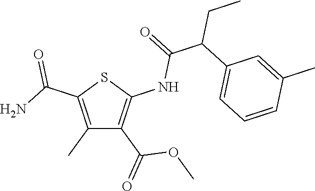 [SMILES: CCC(C(=O)Nc1sc(C(N)=O)c(C)c1C(=O)OC)c1cccc(C)c1] |
| | methyl 5-carbamoyl-4-methyl-2-(2-(m-tolyl)butanamido)thiophene-3-carboxylate |
| 71 | 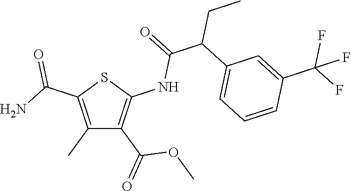 [SMILES: CCC(C(=O)Nc1sc(C(N)=O)c(C)c1C(=O)OC)c1cccc(C(F)(F)F)c1] |
| | methyl 5-carbamoyl-4-methyl-2-(2-(3- |
| | (trifluoromethyl)phenyl)butanamido)thiophene-3-carboxylate |
| 72 | 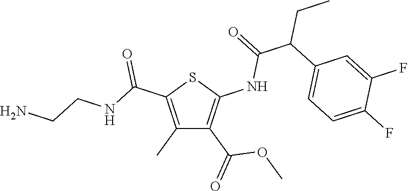 [SMILES: CCC(C(=O)Nc1sc(C(=O)NCCN)c(C)c1C(=O)OC)c1ccc(F)c(F)c1] |
| | methyl 5-((2-aminoethyl)carbamoyl)-2-(2-(3,4-difluorophenyl)butanamido)-4- |
| | methylthiophene-3-carboxylate |
| 73 | 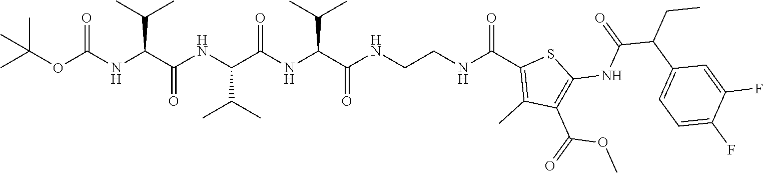 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc(F)c(F)c1] |
| | methyl 2-(2-(3,4-difluorophenyl)butanamido)-4-methyl-5-(((6S,9S,12S)-6,9,12- |
| | triisopropyl-2,2-dimethyl-4,7,10,13-tetraoxo-3-oxa-5,8,11,14-tetraazahexadecan- |
| | 16-yl)carbamoyl)thiophene-3-carboxylate |
| 74 | 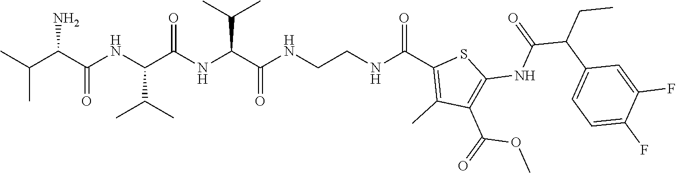 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccc(F)c(F)c1] |
| | methyl 5-((2-((S)-2-((S)-2-((S)-2-amino-3-methylbutanamido)-3- |
| | methylbutanamido)-3-methylbutanamido)ethyl)carbamoyl)-2-(2-(3,4- |
| | difluorophenyl)butanamido)-4-methylthiophene-3-carboxylate |
| 75 | 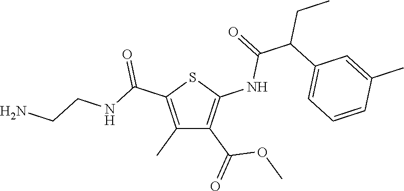 [SMILES: CCC(C(=O)Nc1sc(C(=O)NCCN)c(C)c1C(=O)OC)c1cccc(C)c1] |
| | methyl 5-((2-aminoethyl)carbamoyl)-4-methyl-2-(2-(m- |
| | tolyl)butanamido)thiophene-3-carboxylate |
| 76 | 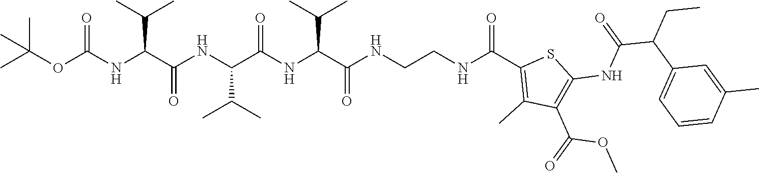 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1cccc(C)c1] |
| | methyl 4-methyl-2-(2-(m-tolyl)butanamido)-5-(((6S,9S,12S)-6,9,12-triisopropyl- |
| | 2,2-dimethyl-4,7,10,13-tetraoxo-3-oxa-5,8,11,14-tetraazahexadecan-16- |
| | yl)carbamoyl)thiophene-3-carboxylate |
| 77 | 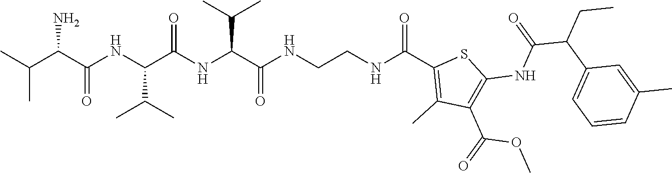 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1cccc(C)c1] |
| | methyl 5-((2-((S)-2-((S)-2-((S)-2-amino-3-methylbutanamido)-3- |
| | methylbutanamido)-3-methylbutanamido)ethyl)carbamoyl)-4-methyl-2-(2-(m- |
| | tolyl)butanamido)thiophene-3-carboxylate |
| 78 | 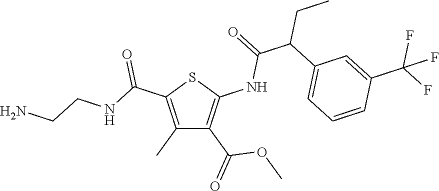 [SMILES: CCC(C(=O)Nc1sc(C(=O)NCCN)c(C)c1C(=O)OC)c1cccc(C(F)(F)F)c1] |
| | methyl 5-((2-aminoethyl)carbamoyl)-4-methyl-2-(2-(3- |
| | (trifluoromethyl)phenyl)butanamido)thiophene-3-carboxylate |
| 79 | 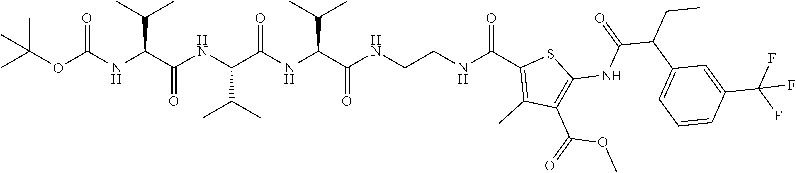 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1cccc(C(F)(F)F)c1] |
| | methyl 4-methyl-2-(2-(3-(trifluoromethyl)phenyl)butanamido)-5-(((6S,9S,12S)- |
| | 6,9,12-triisopropyl-2,2-dimethyl-4,7,10,13-tetraoxo-3-oxa-5,8,11,14- |
| | tetraazahexadecan-16-yl)carbamoyl)thiophene-3-carboxylate |
| 80 | 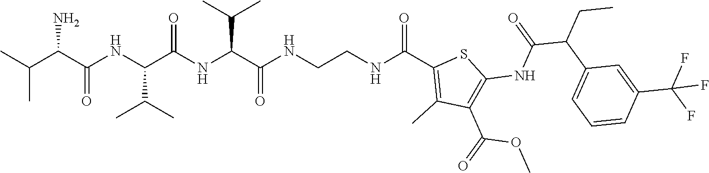 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1cccc(C(F)(F)F)c1] |
| | methyl 5-((2-((S)-2-((S)-2-((S)-2-amino-3-methylbutanamido)-3- |
| | methylbutanamido)-3-methylbutanamido)ethyl)carbamoyl)-4-methyl-2-(2-(3- |
| | (trifluoromethyl)phenyl)butanamido)thiophene-3-carboxylate |
| 81 | 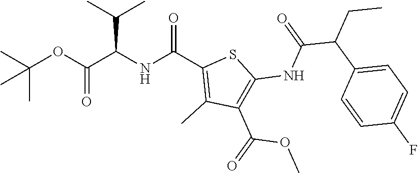 [SMILES: CCC(C(=O)Nc1sc(C(=O)N[C@@H](C(=O)OC(C)(C)C)C(C)C)c(C)c1C(=O)OC)c1ccc(F)cc1] |
| | methyl 5-(((R)-1-(tert-butoxy)-3-methyl-1-oxobutan-2-yl)carbamoyl)-2-(2-(4- |
| | fluorophenyl)butanamido)-4-methylthiophene-3-carboxylate |
| 82 | 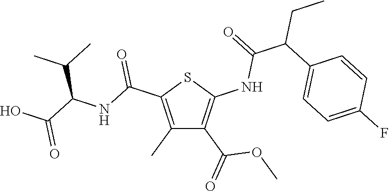 [SMILES: CCC(C(=O)Nc1sc(C(=O)N[C@@H](C(=O)O)C(C)C)c(C)c1C(=O)OC)c1ccc(F)cc1] |
| | (5-(2-(4-fluorophenyl)butanamido)-4-(methoxycarbonyl)-3-methylthiophene-2- |
| | carbonyl)-D-valine |
| 83 | 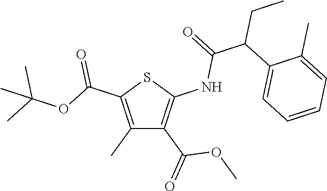 [SMILES: CCC(C(=O)Nc1sc(C(=O)OC(C)(C)C)c(C)c1C(=O)OC)c1ccccc1C] |
| | 2-(tert-butyl) 4-methyl 3-methyl-5-(2-(o-tolyl)butanamido)thiophene-2,4- |
| | dicarboxylate |
| 84 | 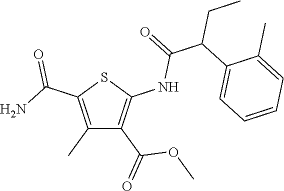 [SMILES: CCC(C(=O)Nc1sc(C(N)=O)c(C)c1C(=O)OC)c1ccccc1C] |
| | methyl 5-carbamoyl-4-methyl-2-(2-(o-tolyl)butanamido)thiophene-3-carboxylate |
| 85 | 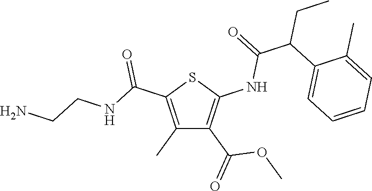 [SMILES: CCC(C(=O)Nc1sc(C(=O)NCCN)c(C)c1C(=O)OC)c1ccccc1C] |
| | methyl 5-((2-aminoethyl)carbamoyl)-4-methyl-2-(2-(o-tolyl)butanamido)thiophene- |
| | 3-carboxylate |
| 86 | 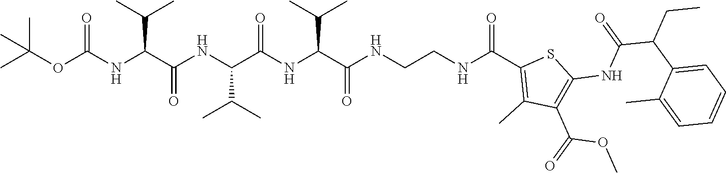 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccccc1C] |
| | methyl 4-methyl-2-(2-(o-tolyl)butanamido)-5-(((6S,9S,12S)-6,9,12-triisopropyl- |
| | 2,2-dimethyl-4,7,10,13-tetraoxo-3-oxa-5,8,11,14-tetraazahexadecan-16- |
| | yl)carbamoyl)thiophene-3-carboxylate |
| 87 | 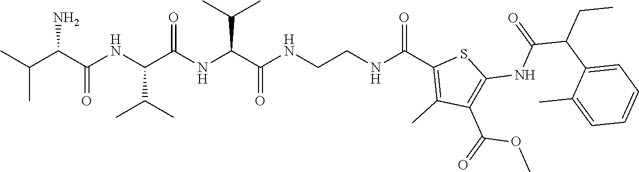 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccccc1C] |
| | methyl 5-((2-((S)-2-((S)-2-((S)-2-amino-3-methylbutanamido)-3- |
| | methylbutanamido)-3-methylbutanamido)ethyl)carbamoyl)-4-methyl-2-(2-(o- |
| | tolyl)butanamido)thiophene-3-carboxylate |
| 88 | 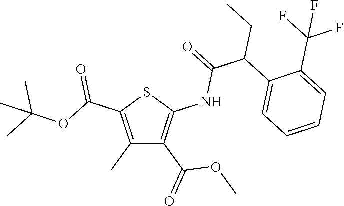 [SMILES: CCC(C(=O)Nc1sc(C(=O)OC(C)(C)C)c(C)c1C(=O)OC)c1ccccc1C(F)(F)F] |
| | 2-(tert-butyl) 4-methyl 3-methyl-5-(2-(2- |
| | (trifluoromethyl)phenyl)butanamido)thiophene-2,4-dicarboxylate |
| 89 | 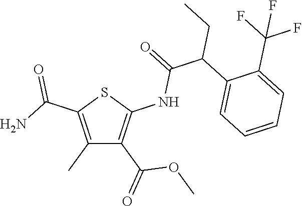 [SMILES: CCC(C(=O)Nc1sc(C(N)=O)c(C)c1C(=O)OC)c1ccccc1C(F)(F)F] |
| | methyl 5-carbamoyl-4-methyl-2-(2-(2- |
| | (trifluoromethyl)phenyl)butanamido)thiophene-3-carboxylate |
| 90 | 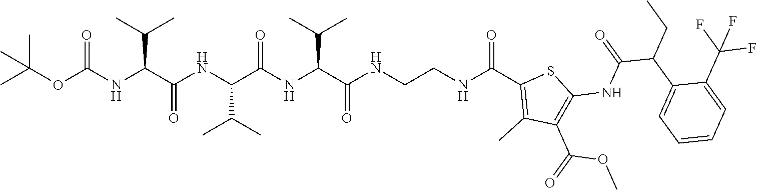 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccccc1C(F)(F)F] |
| | methyl 4-methyl-2-(2-(2-(trifluoromethyl)phenyl)butanamido)-5-(((6S,9S,12S)- |
| | 6,9,12-triisopropyl-2,2-dimethyl-4,7,10,13-tetraoxo-3-oxa-5,8,11,14- |
| | tetraazahexadecan-16-yl)carbamoyl)thiophene-3-carboxylate |
| 91 | 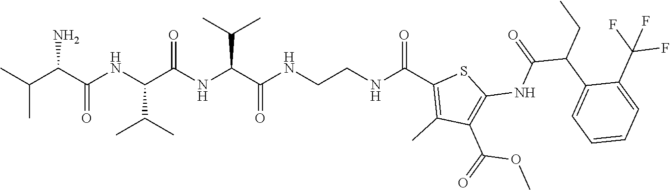 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccccc1C(F)(F)F] |
| | methyl 5-((2-((S)-2-((S)-2-((S)-2-amino-3-methylbutanamido)-3- |
| | methylbutanamido)-3-methylbutanamido)ethyl)carbamoyl)-4-methyl-2-(2-(2- |
| | (trifluoromethyl)phenyl)butanamido)thiophene-3-carboxylate |
| 92 | 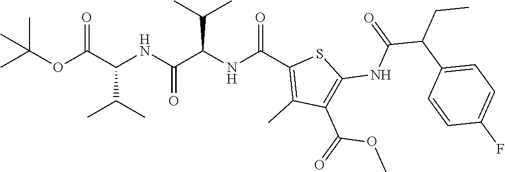 [SMILES: CCC(C(=O)Nc1sc(C(=O)N[C@@H](C(=O)N[C@@H](C(=O)OC(C)(C)C)C(C)C)C(C)C)c(C)c1C(=O)OC)c1ccc(F)cc1] |
| | methyl 5-(((R)-1-(((R)-1-(tert-butoxy)-3-methyl-1-oxobutan-2-yl)amino)-3-methyl- |
| | 1-oxobutan-2-yl)carbamoyl)-2-(2-(4-fluorophenyl)butanamido)-4-methylthiophene- |
| | 3-carboxylate |
| 93 | 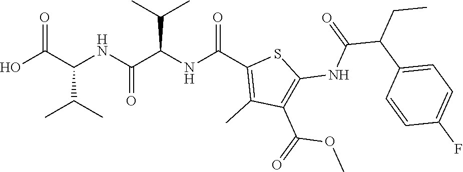 [SMILES: CCC(C(=O)Nc1sc(C(=O)N[C@@H](C(=O)N[C@@H](C(=O)O)C(C)C)C(C)C)c(C)c1C(=O)OC)c1ccc(F)cc1] |
| | (5-(2-(4-fluorophenyl)butanamido)-4-(methoxycarbonyl)-3-methylthiophene-2- |
| | carbonyl)-D-valyl-D-valine |

The compounds described herein can form salts with acids and/or bases, and such salts are included in the present disclosure. In certain other embodiments, the salts are pharmaceutically acceptable salts. The term “salts” embraces addition salts of free acids and/or bases that are useful within the methods of the disclosure. Pharmaceutically unacceptable salts can nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts can be prepared from an inorganic acid or from an organic acid. Examples of inorganic acids include sulfate, hydrogen sulfate, hemisulfate, hydrochloric, hydrobromic, hydriodic, nitric, carbonic, sulfuric, and phosphoric acids (including hydrogen phosphate and dihydrogen phosphate). Appropriate organic acids can be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate).

Suitable pharmaceutically acceptable base addition salts of compounds of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, ammonium, N,N′-dibenzylethylene-diamine, chloroprocaine, choline, diethanolamine, ethylenediamine, meglumine (N-methylglucamine) and procaine.

All of these salts can be prepared from the corresponding compound by reacting, for example, the appropriate acid or base with the compound. Salts can be comprised of a fraction of less than one, one, or more than one molar equivalent of acid or base with respect to any compound of the disclosure. In certain other embodiments, the at least one compound of the disclosure is a component of a pharmaceutical composition further including at least one pharmaceutically acceptable carrier.

The compounds of the disclosure can possess one or more stereocenters, and each stereocenter can exist independently in either the (R) or (S) configuration. In certain other embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain other embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain other embodiments, the compounds described herein exist in solvated forms with pharmaceutically acceptable solvents such as water, and ethanol. In other embodiments, the compounds described herein exist in unsolvated form.

In certain other embodiments, the compounds of the disclosure exist as tautomers. All tautomers are included within the scope of the compounds recited herein.

In certain other embodiments, compounds described herein are prepared as prodrugs. A “prodrug” is an agent converted into the parent drug in vivo. In certain other embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain other embodiments, sites on, for example, the aromatic ring portion of compounds of the disclosure are susceptible to various metabolic reactions. Incorporation of appropriate substituents on the aromatic ring structures can reduce, minimize or eliminate this metabolic pathway. In certain other embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain other embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain other embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and in the art.

General methods for the preparation of compound as described herein are modified by the use of appropriate reagents and conditions, for the introduction of the various moieties found in the formula as provided herein.

### Synthesis

The present disclosure further provides methods of preparing the compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

### Pharmaceutical Compositions

In one aspect, the present disclosure provides a pharmaceutical composition comprising at least one compound of the present disclosure and at least one pharmaceutically acceptable carrier and/or excipient.

### Methods

The disclosure includes methods of treating, ameliorating, and/or preventing an orthopoxvirus infection in a human subject. In certain embodiments, the orthopoxvirus infection is caused by Molluscum contagiosum virus (MCV). In certain embodiments, the orthopoxvirus infection is caused by camelpox virus. In certain embodiments, the orthopoxvirus infection is caused by cowpox virus. In certain embodiments, the orthopoxvirus infection is caused by mousepox virus. In certain embodiments, the orthopoxvirus infection is caused by horsepox virus. In certain embodiments, the orthopoxvirus infection is caused by monkeypox virus. In certain embodiments, the orthopoxvirus infection is caused by raccoonpox virus. In certain embodiments, the orthopoxvirus infection is caused by tanapox virus. In certain embodiments, the orthopoxvirus infection is caused by variola (smallpox virus). In certain embodiments, the orthopoxvirus infection is caused by Yoka poxvirus. In certain embodiments, the orthopoxvirus infection is caused by cervidpoxvirus (deerpox). In certain embodiments, the orthopoxvirus infection is caused by avipoxvirus (fowlpox). In certain embodiments, the orthopoxvirus infection is caused by capripoxvirus (goatpox). In certain embodiments, the orthopoxvirus infection is caused by leporipoxvirus (myxoma virus). In certain embodiments, the orthopoxvirus infection is caused by parapoxvirus (orf virus). In certain embodiments, the orthopoxvirus infection is caused by suipoxvirus (swinepox). In certain embodiments, the orthopoxvirus infection is caused by vatapoxvirus (Yaba-like disease virus). In certain embodiments, the method comprises administering to the subject a therapeutically effective amount of at least one compound of the disclosure, or pharmaceutically acceptable salts, solvates, enantiomers, diastereomers, geometric isomers, or tautomers thereof, or at least one pharmaceutical composition of the present disclosure.

In certain embodiments, folding and/or function of processivity factor mD4 is inhibited in the virus. In certain embodiments, DNA polymerase processivity is disrupted in the virus.

In certain embodiments, the orthopoxvirus infection is caused by a MCV.

In certain embodiments, wherein the subject is a mammal. In certain embodiments, the mammal is a human.

In certain embodiments, the at least one compound and/or pharmaceutical composition is administered topically.

### Administration/Dosage/Formulations

The regimen of administration can affect what constitutes an effective amount. The therapeutic formulations can be administered to the subject either prior to or after the onset of a disease or disorder contemplated in the disclosure. Further, several divided dosages, as well as staggered dosages can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present disclosure to a patient, preferably a mammal, more preferably a human, can be carried out using known procedures, at dosages and for periods of time effective to treat a disease or disorder contemplated in the disclosure. An effective amount of the therapeutic compound necessary to achieve a therapeutic effect can vary according to factors such as the state of the disease or disorder in the patient; the age, sex, and weight of the patient; and the ability of the therapeutic compound to treat a disease or disorder contemplated in the disclosure. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the disclosure is from about 1 and 5,000 mg/kg of body weight/per day. The pharmaceutical compositions useful for practicing the disclosure can be administered to deliver a dose of from 1 ng/kg/day and 100 mg/kg/day. One of ordinary skill in the art would be able to study the relevant factors and make the determination regarding the effective amount of the therapeutic compound without undue experimentation.

A medical doctor, e.g., physician or veterinarian, having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

In certain other embodiments, the compositions of the disclosure are formulated using one or more pharmaceutically acceptable excipients or carriers. In other embodiments, the pharmaceutical compositions of the disclosure comprise a therapeutically effective amount of a compound of the disclosure and a pharmaceutically acceptable carrier. In yet other embodiments, the compound of the disclosure is the only biologically active agent (i.e., capable of treating, ameliorating, and/or preventing diseases and disorders discussed herein) in the composition. In yet other embodiments, the compound of the disclosure is the only biologically active agent (i.e., capable of treating, ameliorating, and/or preventing diseases and disorders discussed herein) in therapeutically effective amounts in the composition.

In certain other embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration can be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 300 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therein between.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain other embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated in the disclosure.

Formulations can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents.

Routes of administration of any of the compositions of the disclosure include intravitreal, oral, nasal, rectal, intravaginal, parenteral, buccal, sublingual or topical. The compounds for use in the disclosure can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravitreal, intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intravitreal, intraperitoneal, intramuscular, intrasternal injection, and kidney dialytic infusion techniques.

### Topical Administration

An obstacle for topical administration of pharmaceuticals is the stratum comeum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum comeum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient can be as high as the solubility limit of the active ingredient in the solvent. Formulations for topical administration can further comprise one or more of the additional ingredients described herein.

Enhancers of permeation can be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure can contain liposomes. The composition of the liposomes and their use are known in the art (for example, see U.S. Pat. No. 6,323,219).

In alternative embodiments, the topically active pharmaceutical composition can be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum comeum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition can further comprise a hydrotropic agent, which functions to increase disorder in the structure of the stratum comeum, and thus allows increased transport across the stratum comeum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds can be synthetically- or naturally derived.

### Buccal Administration

A pharmaceutical composition of the disclosure can be prepared, packaged, or sold in a formulation suitable for buccal administration. Such formulations may, for example, be in the form of tablets or lozenges made using conventional methods, and can contain, for example, 0.1 to 20% (w/w) of the active ingredient, the balance comprising an orally dissolvable or degradable composition and, optionally, one or more of the additional ingredients described herein. Alternately, formulations suitable for buccal administration can comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and can further comprise one or more of the additional ingredients described herein. The examples of formulations described herein are not exhaustive and it is understood that the disclosure includes additional modifications of these and other formulations not described herein, but which are known to those of skill in the art.

### Controlled Release Formulations and Drug Delivery Systems

In certain other embodiments, the formulations of the present disclosure can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form. In certain embodiments, the compounds of the disclosure can be formulated for sustained release over a period of 3-12 months.

For sustained release, the compounds can be formulated with a suitable polymer or hydrophobic material that provides sustained release properties to the compounds. As such, the compounds useful within the methods of the disclosure can be administered in the form of microparticles, for example by injection, or in the form of wafers or discs by implantation.

In one embodiment of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present disclosure depends on the age, sex and weight of the patient, the current medical condition of the patient and the progression of a disease or disorder contemplated in the disclosure. The skilled artisan is able to determine appropriate dosages depending on these and other factors.

A suitable dose of a compound of the present disclosure can be in the range of from about 0.01 mg to about 5,000 mg per day, such as from about 0.1 mg to about 1,000 mg, for example, from about 1 mg to about 500 mg, such as about 5 mg to about 250 mg per day.

The dose can be administered in a single dosage or in multiple dosages, for example from 1 to 5 or more times per day. When multiple dosages are used, the amount of each dosage can be the same or different. For example, a dose of 1 mg per day can be administered as two 0.5 mg doses, with about a 12-hour interval between doses.

It is understood that the amount of compound dosed per day can be administered, in non-limiting examples, every day, every other day, every 2 days, every 3 days, every 4 days, or every 5 days.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain other embodiments, patients require intermittent treatment on a long-term basis upon any recurrence of symptoms and/or infection.

The compounds for use in the method of the disclosure can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 5 or more times per day). When multiple daily doses are used, the unit dosage form can be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Example 1: Compound Synthesis

### Synthesis of Core Intermediate(s)

### Methyl 5-((2-aminoethyl)carbamoyl)-4-methyl-2-(2-(4-(trifluoromethyl)phenyl)butanamido)thiophene-3-carboxylate

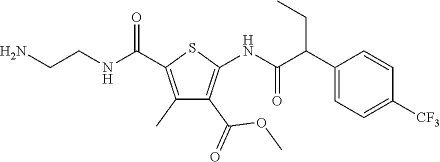 [SMILES: CCC(C(=O)Nc1sc(C(=O)NCCN)c(C)c1C(=O)OC)c1ccc(C(F)(F)F)cc1]

### Step (1): 2-(4-(Trifluoromethyl)phenyl)butanoic acid

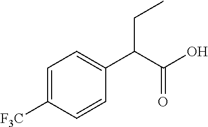 [SMILES: CCC(C(=O)O)c1ccc(C(F)(F)F)cc1]

Diisopropylamnne (1.95 g, 19.3 mmol) was dissolved in anhydrous tetrahydrofuran. The solution was cooled to −78° C. using a dry ice/acetone bath, and n-butyllithium (2.5 M in hexanes, 7.70 mL, 19.3 mmol) was added slowly. The solution was stirred for 1 hour at −78° C. Ethyl 4-(trifluoromethyl)phenylacetate (4.47 g, 19.3 mmol) was added. The solution was stirred for 1 hour at −78° C., then ethyl iodide (3.00 g, 19.3 mmol) was added. The cooling bath was removed and the reaction was allowed to warm to room temperature and was stirred overnight. The reaction was then diluted with saturated aqueous sodium bicarbonate and extracted with ethyl acetate. The extracts were concentrated and chromatographed (120 g silica column; hexanes/ethyl acetate) to give 3.64 g of ethyl 2-(4-(trifluoromethyl)phenyl)butanoate as an off-white solid.

This material was dissolved in tetrahydrofuran and lithium hydroxide hydrate (1.18 g, 28 mmol) was added. To the suspension, was added water until the solid was mostly dissolved, then the reaction was stirred overnight at room temperature. The reaction was acidified with 1N hydrochloric acid and extracted with ethyl acetate. The extracts were concentrated to provide the title compound as a white solid (3.14 g). ¹H NMR (300 MHz, CDCl₃) δ: 7.35 (2H, d, J=8.6 Hz), 7.20 (2H, d, J=8.6 Hz), 3.90 (1H, t, J=7.6 Hz), 1.69-1.84 (m, 2H), 0.91 (3H, t, J=7.6 Hz).

### Step (2): 4-(Methoxycarbonyl)-3-methyl-S-(2-(4-(trifluoromethyl)phenyl)butanamido)thiophene-2-carboxylic acid

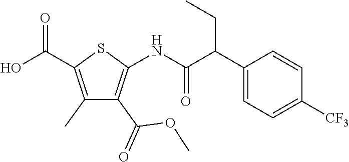 [SMILES: CCC(C(=O)Nc1sc(C(=O)O)c(C)c1C(=O)OC)c1ccc(C(F)(F)F)cc1]

2-(4-(Trifluoromethyl)phenyl)butanoic acid (3.14 g, 13.6 mmol) was dissolved in anhydrous dichloromethane, then oxalyl chloride (2.0 M in dichloromethane, 6.78 mL, 13.6 mmol) and dimethylformamide (5 drops, catalytic) were added. After 2 hours at room temperature, the reaction was concentrated in vacuo. The residue was taken up in anhydrous pyridine (25 mL) and 2-(tert-butyl) 4-methyl 5-amino-3-methylthiophene-2,4-dicarboxylate (3.68 g, 13.6 mmol) was added. The mixture was stirred for 3 days at room temperature then diluted with brine and extracted with ethyl acetate. The extracts were concentrated and then dissolved in 4 N hydrogen chloride in dioxane (75 mL). The reaction was stirred at room temperature overnight then concentrated and chromatographed (120 g silica column; hexanes/ethyl acetate) to provide the title compound as an off-white solid (2.10 g). MS m/z 430.42 (M+H). ¹H NMR (300 MHz, CDCl₃) δ: 11.11 (s, 1H), 7.22 (2H, d, J=8.6 Hz), 7.13 (2H, d, J=8.6 Hz), 6.23 (br s, 1H), 4.81 (m, 1H), 3.43 (s, 3H), 1.81 (m, 2H), 0.95 (m, 3H).

### Step (3): Methyl 5-((2-aminoethyl)carbamoyl)-4-methyl-2-(2-(4-(trifluoromethyl)phenyl)butanamido)thiophene-3-carboxylate

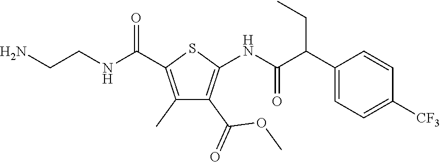 [SMILES: CCC(C(=O)Nc1sc(C(=O)NCCN)c(C)c1C(=O)OC)c1ccc(C(F)(F)F)cc1]

4-(Methoxycarbonyl)-3-methyl-5-(2-(4-(trifluoromethyl)phenyl)butanamido)thiophene-2-carboxylic acid (384 mg, 0.90 mmol) was dissolved in anhydrous dichloromethane, then oxalyl chloride (2.0 M in dichloromethane, 0.450 mL, 0.90 mmol) and dimethylformamide (5 drops, catalytic) were added. After one hour, diisopropylethylamine (289 mg, 2.24 mmol) and tert-butyl (2-aminoethyl)carbamate (173 mg, 1.08 mmol) were added. The reaction was stirred overnight at room temperature.

The reaction was then diluted with saturated aqueous sodium bicarbonate and extracted with dichloromethane. The extracts were concentrated and chromatographed (40 g silica column: hexanes/ethyl acetate) to provide methyl 5-((2-((tert-butoxycarbonyl)amino)ethyl)carbamoyl)-4-methyl-2-(2-(4-(trifluoromethyl)phenyl)butanamido)thiophene-3-carboxylate (345 mg). This material was dissolved in 4 N hydrochloric acid in dioxane (20 mL) and stirred overnight. The solution was the lyophilized to provide the hydrochloride salt of the title compound as a light tan solid (345 mg). MS m/z 472.42 (M+H). ¹H NMR (300 MHz, CDCl₃) δ: 11.23 (s, 1H), 8.22 (br s, 2H), 7.80 (br s, 1H), 7.45-7.60 (m, 2H), 7.38-7.41 (m, 2H) 3.20-7.95 (m, 8H) 2.12-2.47 (m, 4H), 0.68-0.90 (m, 3H).

In view of the present disclosure and U.S. Patent Application No. 63/248,670, which is hereby incorporated by reference in its entirety, one skilled in the art of synthetic chemistry would be enabled to prepare any of a number of alternate core intermediates, including but not limited to methyl 5-carbamoyl-4-methyl-2-(2-phenylbutanamido)thiophene-3-carboxylate (FIG. 4).

### Conjugated Compounds

### Methyl 5-((2(2-((tert-butoxycarbonyl)amino)acetamido)ethyl)carbamoyl)-2-(2-(4-fluorophenyl)butanamido)-4-methylthiophene-3-carboxylate (1)

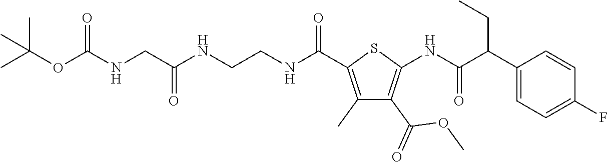 [SMILES: CCC(C(=O)Nc1sc(C(=O)NCCNC(=O)CNC(=O)OC(C)(C)C)c(C)c1C(=O)OC)c1ccc(F)cc1]

Methyl 5-(2-aminoethylcarbamoyl)-2-(2-(4-fluorophenyl)butanamido)-4-methylthiophene-3-carboxylate trifluoroacetate (250 mg, 0.47 mmol), N-Boc glycine (123 mg, 0.70 mmol), DIEA (245 μL, 1.41 mmol) and HATU (357 mg, 0.94 mmol) were taken up into 2 mL dimethylformamide. The reaction was stirred overnight at room temperature. Ethyl acetate was added, then the mixture washed with water and brine. The solvent was removed in vacuo to provide the crude product (260 mg), which was used in the subsequent reaction without further purification. A 55 mg portion of the crude product was purified by preparative HPLC to provide the title compound (42 mg). MS m/z 579.73 (M+H). ¹H NMR (300 MHz, methanol-d₄) δ: 7.39 (dd, J=5.6, 8.5 Hz, 2H), 7.20-7.01 (m, 2H), 3.84 (s, 3H), 3.79-3.61 (m, 4H), 3.48-3.35 (m, 4H), 2.52 (s, 3H), 2.21 (quind, J=7.3, 14.1 Hz, 1H), 1.97-1.76 (m, 1H), 1.41 (s, 9H), 0.91 (t, J=7.3 Hz, 3H).

### Methyl 5-((2(2-aminoacetamido)ethyl)carbamoyl)-2-(2-(4-fluorophenyl)butanamido)-4-methylthiophene-3-carboxylate (2)

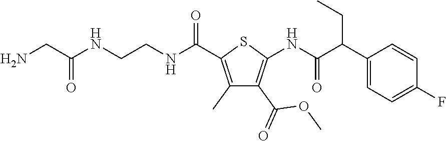 [SMILES: CCC(C(=O)Nc1sc(C(=O)NCCNC(=O)CN)c(C)c1C(=O)OC)c1ccc(F)cc1]

Methyl 5-((2-(2-((tert-butoxycarbonyl)amino)acetamido)ethyl)carbamoyl)-2-(2-(4-fluorophenyl)butanamido)-4-methylthiophene-3-carboxylate (260 mg, 0.45 mmol) was dissolved in dichloromethane (2 mL) and trifluoroacetic acid (1 mL), and the reaction was stirred for 1 hr. The solvent was removed in vacuo and the crude material was purified by preparative HPLC to provide the title compound as a trifluoroacetate salt (152 mg, 58% yield). MS m/z 479.62 (M+H). ¹H NMR (300 MHz, methanol-d₄) δ: 7.54-7.28 (m, 2H), 7.22-6.96 (m, 2H), 3.85 (s, 3H), 3.78-3.70 (m, 1H), 3.65 (d, J=1.2 Hz, 3H), 3.45 (d, J=2.3 Hz, 4H), 2.53 (s, 3H), 2.20 (dd, J=7.0, 14.1 Hz, 1H), 2.01-1.77 (m, 1H), 0.91 (t, J=7.3 Hz, 3H).

### Methyl 5-((2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate (3)

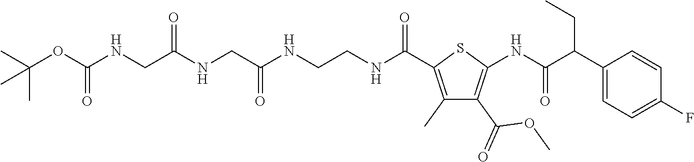 [SMILES: CCC(C(=O)Nc1sc(C(=O)NCCNC(=O)CNC(=O)CNC(=O)OC(C)(C)C)c(C)c1C(=O)OC)c1ccc(F)cc1]

Methyl 5-((2-(2-aminoacetamido)ethyl)carbamoyl)-2-(2-(4-fluorophenyl)butanamido)-4-methylthiophene-3-carboxylate trifluoroacetate (135 mg, 0.23 mmol), N-Boc glycine (51 mg, 0.29 mmol), diisopropylethylamine (120 μL, 0.69 mmol) and HATU (131 mg, 0.35 mmol) were dissolved in dimethylformamide (1 mL) and the reaction was stirred overnight at room temperature. Ethyl acetate was added, and the mixture was washed with water and brine. The solvent was removed in vacuo to provide the crude product which was purified by preparative HPLC to provide the title compound (98 mg, 56% yield). MS m/z 636.78 (M+H). ¹H NMR (300 MHz, methanol-d₄) δ: 7.40 (dd, J=5.6, 8.5 Hz, 2H), 7.11 (t, J=8.5 Hz, 2H), 3.84 (s, 5H), 3.78-3.70 (m, 3H), 3.65 (s, 2H), 3.42 (dd, J=3.8, 9.1 Hz, 4H), 2.52 (s, 3H), 2.33-2.09 (m, 1H), 2.03-1.78 (m, 1H), 1.42 (s, 9H), 0.91 (t, J=7.6 Hz, 3H).

### Methyl 5-((2-((S)-2-amino-3-methylbutanamido)ethyl)carbamoyl)-2-(2-(4-fluorophenyl)butanamido)-4-methylthiophene-3-carboxylate (4)

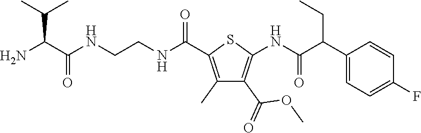 [SMILES: CCC(C(=O)Nc1sc(C(=O)NCCNC(=O)[C@@H](N)C(C)C)c(C)c1C(=O)OC)c1ccc(F)cc1]

Methyl 5-((2-aminoethyl)carbamoyl)-2-(2-(4-fluorophenyl)butanamido)-4-methylthiophene-3-carboxylate trifluoroacetate (230 mg, 0.43 mmol), N-Boc-L-valine (131 mg, 0.65 mmol), diisopropylethylamine (224 μL, 1.29 mmol) and HATU (327 mg, 0.86 mmol) were taken up into 2 mL dimethylformamide, and the reaction was stirred overnight at room temperature. Ethyl acetate was added, and the mixture was washed with water and brine. The solvent was removed in vacuo and crude material was purified by normal phase column chromatography, using a 0-10% (MeOH/dichloromethane eluent gradient. Next, 260 mg of isolated material was added to a 1:2 mixture of trifluoroacetic acid and dichloromethane (3 mL). The mixture was stirred for 1 hr and the solvent was removed in vacuo. The material was purified by preparative HPLC to provide the title compound as a trifluoroacetate salt (182 mg, 65% yield). MS m/z 521.65 (M+H). ¹H NMR (300 MHz, methanol-d₄) δ: 7.53-7.25 (m, 2H), 7.21-7.01 (m, 2H), 3.85 (s, 3H), 3.74 (s, 1H), 3.59 (d, J=5.3 Hz, 1H), 3.57-3.36 (m, 4H), 2.54 (s, 3H), 2.28-2.11 (m, 2H), 2.02-1.74 (m, 1H), 1.03 (t, J=7.6 Hz, 6H), 0.91 (t, J=7.3 Hz, 3H).

### Methyl 5-(((6S,9S)-6,9-diisopropyl-2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate (5)

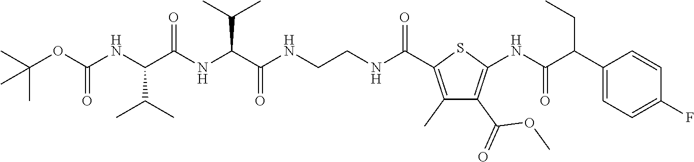 [SMILES: CCC(C(=O)Nc1sc(C(=O)NCCNC(=O)[C@@H](NC(=O)[C@@H](NC(=O)OC(C)(C)C)C(C)C)C(C)C)c(C)c1C(=O)OC)c1ccc(F)cc1]

Methyl 5-((2-(2-aminoacetamido)ethyl)carbamoyl)-2-(2-(4-fluorophenyl)butanamido)-4-methylthiophene-3-carboxylate trifluoroacetate (90 mg, 0.14 mmol), N-Boc-L-valine (43 mg, 0.21 mmol), diisopropylethylamine (73 μL, 0.42 mmol) and HATU (106 mg, 0.28 mmol) were taken up into 2 mL dimethylformamide, and the reaction was stirred overnight at room temperature. Ethyl acetate was added, and the mixture was washed with water and brine. The solvent was removed in vacuo and crude material was purified by preparative HPLC to provide the title compound (52 mg, 51% yield). MS m/z 720.91 (M+H). ¹H NMR (300 MHz, methanol-d₄) δ: 7.40 (dd, J=5.3, 8.8 Hz, 2H), 7.11 (t, J=8.8 Hz, 2H), 4.09 (s, 1H), 3.88-3.83 (m, 4H), 3.78-3.63 (m, 2H), 3.49-3.35 (m, 4H), 2.53 (s, 3H), 2.36-1.75 (m, 4H), 1.42 (s, 9H), 1.01-0.78 (m, 16H).

### Methyl 5-((2-(2-(2-aminoacetamido)acetamido)ethyl)carbamoyl)-2-(2-(4-fluorophenyl)butanamido)-4-methylthiophene-3-carboxylate (6)

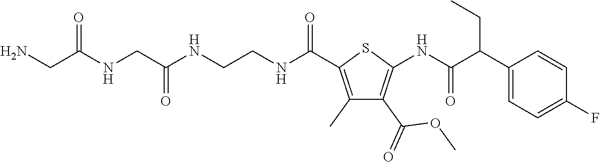 [SMILES: CCC(C(=O)Nc1sc(C(=O)NCCNC(=O)CNC(=O)CN)c(C)c1C(=O)OC)c1ccc(F)cc1]

Methyl 5-((2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate (88 mg, 0.14 mmol) was taken up into 2 mL dichloromethane and 1 mL of trifluoroacetic acid and the reaction was stirred for 1 hr. The solvent was removed in vacuo and the crude material was purified by preparative HPLC to provide the title compound as a trifluoroacetate salt (80 mg, 86% yield). MS m/z 536.68 (M+H). ¹H NMR (300 MHz, methanol-d₄) δ: 7.40 (dd, J=5.3, 8.8 Hz, 2H), 7.11 (t, J=8.8 Hz, 2H), 3.94-3.81 (m, 5H), 3.76 (s, 3H), 3.43 (dd, J=5.3, 12.9 Hz, 4H), 2.53 (s, 3H), 2.29-2.09 (m, 1H), 2.04-1.77 (m, 1H), 0.91 (t, J=7.3 Hz, 3H).

### Methyl 5-((2-((S)-2-((S)-2-amino-3-methylbutanamido)-3-methylbutanamido)ethyl)carbamoyl)-2-(2-(4-fluorophenyl)butanamido)-4-methylthiophene-3-carboxylate (7)

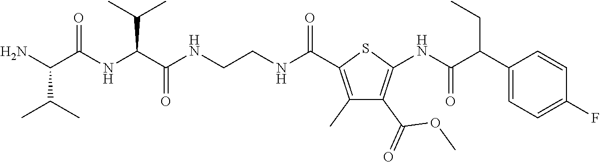 [SMILES: CCC(C(=O)Nc1sc(C(=O)NCCNC(=O)[C@@H](NC(=O)[C@@H](N)C(C)C)C(C)C)c(C)c1C(=O)OC)c1ccc(F)cc1]

Methyl 5-(((6S,9S)-6,9-diisopropyl-2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate (70 mg, 0.10 mmol) was taken up into 1 mL dichloromethane and 0.5 mL of trifluoroacetic acid, and the reaction was stirred for 1 hr. The solvent was removed in vacuo and placed under high vacuum overnight to provide the title compound as a trifluoroacetate salt (75 mg, 93% yield). MS m/z 620.57 (M+H). ¹H NMR (300 MHz, methanol-d₄) δ: 7.40 (dd, J=5.6, 8.5 Hz, 2H), 7.11 (t, J=8.8 Hz, 2H), 4.20-4.04 (m, 1H), 3.85 (s, 3H), 3.80-3.60 (m, 4H), 3.51-3.34 (m, 4H), 2.54 (s, 3H), 2.33-1.96 (m, 3H), 1.95-1.77 (m, 1H), 1.05-0.78 (m, 15H).

### Methyl 2-(2-(4-fluorophenyl)butanamido)-4-methyl-S-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate (8)

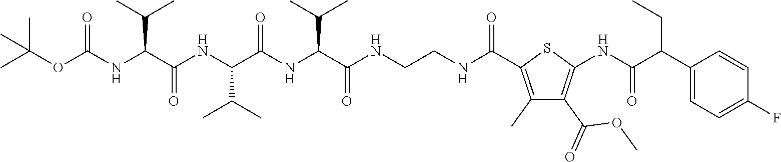 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc(F)cc1]

Methyl 5-((2-((S)-2-amino-3-methylbutanamido)ethyl)carbamoyl)-2-(2-(4-fluorophenyl)butanamido)-4-methylthiophene-3-carboxylate trifluoroacetate (90 mg, 0.14 mmol), N-Boc-L-Val-Val (66 mg, 0.21 mmol), diisopropylethylamine (73 μL, 0.42 mmol) and HATU (106 mg, 0.28 mmol) were taken up into 1 mL dimethylformamide, and the reaction was stirred overnight at room temperature. Ethyl acetate was added, and the mixture was washed with water and brine. The solvent was removed in vacuo and the crude material was purified by preparative HPLC to provide the title compound (30 mg, 26% yield). MS m/z 819.92 (M+H). ¹H NMR (300 MHz, methanol-d₄) δ: 7.40 (dd, J=5.3, 8.8 Hz, 2H), 7.19-6.99 (m, 2H), 4.23-4.01 (m, 2H), 3.90-3.79 (m, 4H), 3.78-3.62 (m, 2H), 3.42 (d, J=5.3 Hz, 4H), 2.53 (s, 3H), 2.29-2.11 (m, 1H), 2.09-1.75 (m, 4H), 1.43 (s, 10H), 0.99-0.80 (m, 21H).

### Methyl 5-((2-((S)-2-((S)-2-((S)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate (9)

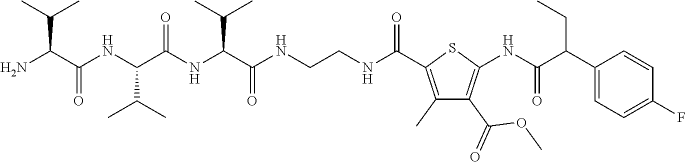 [SMILES: CCC(C(=O)Nc1sc(C(=O)NCCNC(=O)[C@@H](NC(=O)[C@@H](NC(=O)[C@@H](N)C(C)C)C(C)C)C(C)C)c(C)c1C(=O)OC)c1ccc(F)cc1]

Methyl 2-(2-(4-fluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate (20 mg, 0.024 mmol) was taken up into 1 mL dichloromethane and 0.5 mL of trifluoroacetic acid, and the reaction was stirred for 1 hr. The solvent was removed in vacuo and the crude material was placed under high vacuum overnight to provide the title compound as a trifluoroacetate salt (18 mg, 95% yield). ¹H NMR (300 MHz, methanol-d₄) δ: 7.45-7.26 (m, 2H), 7.19-6.94 (m, 2H), 4.25-3.95 (m, 2H), 3.86-3.75 (m, 3H), 3.68 (d, J=6.4 Hz, 2H), 3.35 (d, J=4.1 Hz, 3H), 2.60-2.42 (m, 3H), 2.29-1.67 (m, 5H), 1.05-0.74 (m, 19H).

### Methyl 5-(((5S,8S,11S,14S)-14-amino-5,8,11-triisopropyl-15-methyl-4,7,10,13-tetraoxo-3,6,9,12-tetraazahexadecyl)carbamoyl)-2-(2-(4-fluorophenyl)butanamido)-4-methylthiophene-3-carboxylate (10)

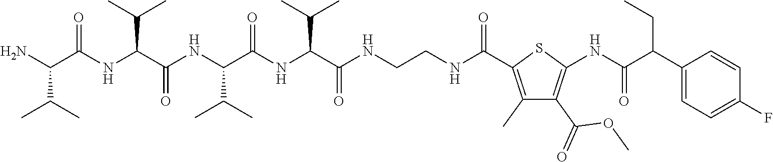 [SMILES: CCC(C(=O)Nc1sc(C(=O)NCCNC(=O)[C@@H](NC(=O)[C@@H](NC(=O)[C@@H](NC(=O)[C@@H](N)C(C)C)C(C)C)C(C)C)C(C)C)c(C)c1C(=O)OC)c1ccc(F)cc1]

Methyl 5-((2-((S)-2-((S)-2-amino-3-methylbutanamido)-3-methylbutanamido)ethyl)carbamoyl)-2-(2-(4-fluorophenyl)butanamido)-4-methylthiophene-3-carboxylate trifluoroacetate (150 mg, 0.2 mmol), N-Boc-L-val-val (80 mg, 0.25 mmol), diisopropylethylamine (174 μL, 1.0 mmol) and HATU (95 mg, 0.25 mmol) were taken up into 2 mL dimethylformamide, and the reaction was stirred overnight at room temperature.

Water was added and the product was filtered off to provide crude intermediate (32 mg) which was added to a 1:2 mixture of trifluoroacetic acid/dichloromethane (2 mL). The mixture was stirred for 1 hr, and solvent was removed in vacuo to provide the crude product. The crude product was purified by preparative HPLC to provide the title compound as a trifluoroacetate salt (18 mg, 10% yield). MS m/z 818.83 (M+H). ¹H NMR (300 MHz, methanol-d₄) δ: 7.36 (dd, J=5.6, 8.5 Hz, 2H), 7.20-6.96 (m, 2H), 4.26-4.13 (m, 2H), 4.06 (t, J=8.2 Hz, 1H), 3.81 (s, 3H), 3.77-3.64 (m, 2H), 3.46-3.30 (m, 4H), 2.49 (s, 3H), 2.27-1.77 (m, 7H), 1.39-1.39 (m, 1H), 1.08-0.69 (m, 27H).

### Methyl 5-((2,2-dimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate (11)

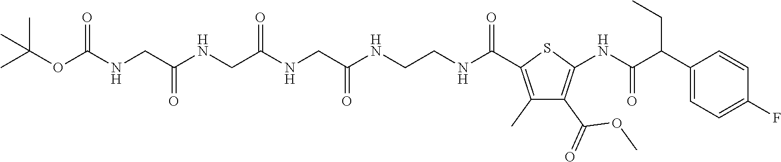 [SMILES: CCC(C(=O)Nc1sc(C(=O)NCCNC(=O)CNC(=O)CNC(=O)CNC(=O)OC(C)(C)C)c(C)c1C(=O)OC)c1ccc(F)cc1]

Methyl 5-((2-(2-(2-aminoacetamido)acetamido)ethyl)carbamoyl)-2-(2-(4-fluorophenyl)butanamido)-4-methylthiophene-3-carboxylate trifluoroacetate (80 mg, 0.12 mmol), N-Boc glycine (27 mg, 0.15 mmol), diisopropylethylamine (84 μL, 0.48 mmol) and HATU (57 mg, 0.15 mmol) were taken up into 1 mL dimethylformamide and the reaction was stirred overnight at room temperature. Ethyl acetate was added and the mixture was washed with water and brine. The solvent was removed in vacuo and to obtain the crude product which was purified by normal phase chromatography using a 0-10% MeOH/dichloromethane eluent gradient to provide the title compound (81 mg, 92% yield). MS m/z 693.68 (M+H). ¹H NMR (300 MHz, methanol-d₄) δ: 7.50-7.32 (m, 2H), 7.20-7.05 (m, 2H), 3.90 (s, 2H), 3.85 (s, 5H), 3.78-3.68 (m, 4H), 3.50-3.35 (m, 4H), 2.53 (s, 3H), 2.20 (dd, J=7.0, 14.1 Hz, 1H), 1.88 (td, J=7.8, 13.6 Hz, 1H), 1.47-1.47 (m, 1H), 1.44-1.43 (m, 1H), 1.47-1.39 (m, 11H), 0.98-0.84 (m, 3H).

### Methyl 5-((2-(2-(2-(2-aminoacetamido)acetamido)acetamido)ethyl)carbamoyl)-2-(2-(4-fluorophenyl)butanamido)-4-methylthiophene-3-carboxylate (12)

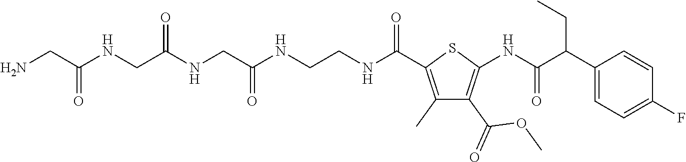 [SMILES: CCC(C(=O)Nc1sc(C(=O)NCCNC(=O)CNC(=O)CNC(=O)CN)c(C)c1C(=O)OC)c1ccc(F)cc1]

Methyl 5-((2,2-dimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate (60 mg, 0.087 mmol) was taken up into 1 mL dichloromethane and 0.5 mL of trifluoroacetic acid and the reaction was stirred for 1 hr. The solvent was removed in vacuo and placed under high vacuum overnight to provide the title compound as a trifluoroacetate salt (65 mg, 86% yield). MS m/z 593.53 (M+H). ¹H NMR (300 MHz, methanol-d₄) δ: 7.49-7.30 (m, 2H), 7.23-7.03 (m, 2H), 3.98 (s, 2H), 3.87-3.84 (m, 5H), 3.76 (s, 3H), 3.43 (dd, J=5.3, 11.7 Hz, 4H), 2.53 (s, 3H), 2.30-2.12 (m, 1H), 1.97-1.78 (m, 1H), 0.97-0.85 (m, 3H).

### Methyl 5-((2,2-dimethyl-4,7,10,13,16-pentaoxo-3-oxa-5,8,11,14,17-pentaazanonadecan-19-yl)carbamoyl)-2-(2-(4-fluorophenyl)butanamido)-4-methylthiophene-3-carboxylate (13)

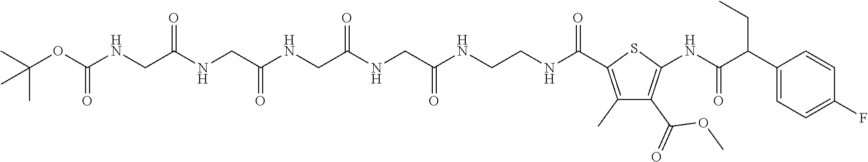 [SMILES: CCC(C(=O)Nc1sc(C(=O)NCCNC(=O)CNC(=O)CNC(=O)CNC(=O)CNC(=O)OC(C)(C)C)c(C)c1C(=O)OC)c1ccc(F)cc1]

Methyl 5-((2-(2-(2-aminoacetamido)acetamido)ethyl)carbamoyl)-2-(2-(4-fluorophenyl)butanamido)-4-methylthiophene-3-carboxylate trifluoroacetate (60 mg, 0.08 mmol), N-Boc glycine (18 mg, 0.10 mmol), diisopropylethylamine (56 μL, 0.32 mmol) and HATU (38 mg, 0.10 mmol) were taken up into 1 mL dimethylformamide and the reaction was stirred overnight at room temperature. Ethyl acetate was added, and the mixture was washed with water and brine. The solvent was removed in vacuo and the crude material was purified by preparative HPLC to provide the title compound (30 mg, 50% yield). MS m/z 750.69 (M+H). ¹H NMR (300 MHz, methanol-d₄) δ: 7.41 (br. s., 2H), 7.12 (br. s., 2H), 3.95-3.82 (m, 9H), 3.76 (br. s., 3H), 3.45 (d, J=7.6 Hz, 4H), 2.54 (br. s., 3H), 2.34-2.12 (m, 1H), 2.04-1.80 (m, 1H), 1.44 (br. s., 8H), 1.02-0.81 (m, 3H).

### Methyl 5-((14-amino-4,7,10,13-tetraoxo-3,6,9,12-tetraazatetradecyl)carbamoyl)-2-(2-(4-fluorophenyl)butanamido)-4-methylthiophene-3-carboxylate (14)

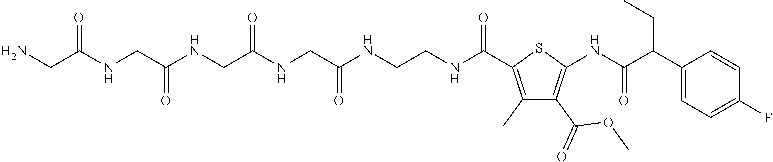 [SMILES: CCC(C(=O)Nc1sc(C(=O)NCCNC(=O)CNC(=O)CNC(=O)CNC(=O)CN)c(C)c1C(=O)OC)c1ccc(F)cc1]

Methyl 5-((2,2-dimethyl-4,7,10,13,16-pentaoxo-3-oxa-5,8,11,14,17-pentaazanonadecan-19-yl)carbamoyl)-2-(2-(4-fluorophenyl)butanamido)-4-methylthiophene-3-carboxylate (21 mg, 0.028 mmol) was taken up into 1 mL dichloromethane and 0.5 mL of trifluoroacetic acid, and the reaction was stirred for 1 hr. The solvent was removed in vacuo and the crude material was placed under high vacuum overnight to provide the title compound as a trifluoroacetate salt (20 mg, 93% yield). MS m/z 650.67 (M+H). ¹H NMR (300 MHz, methanol-d₄) δ: 7.48-7.31 (m, 2H), 7.24-6.97 (m, 2H), 4.07-3.66 (m, 13H), 3.43 (dd, J=5.0, 11.4 Hz, 4H), 2.53 (s, 3H), 2.21 (td, J=7.0, 14.1 Hz, 1H), 1.92 (d, J=8.2 Hz, 1H), 0.91 (t, J=7.3 Hz, 3H).

### Methyl 5-((2-((S)-2-amino-4-methylpentanamido)ethyl)carbamoyl)-2-(2-(4-fluorophenyl)butanamido)-4-methylthiophene-3-carboxylate (15)

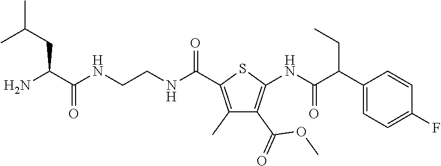 [SMILES: CCC(C(=O)Nc1sc(C(=O)NCCNC(=O)[C@@H](N)CC(C)C)c(C)c1C(=O)OC)c1ccc(F)cc1]

Boc-leucine (21 mg, 0.092 mmol), PyBop (48 mg, 0.092 mmol), and diisopropylethylamine (49 mg, 0.38 mmol) were combined in anhydrous dimethylformamide (1 mL). The mixture was stirred for 15 minutes and then methyl 5-((2-aminoethyl)carbamoyl)-2-(2-(4-fluorophenyl)butanamido)-4-methylthiophene-3-carboxylate hydrochloride (35 mg, 0.076 mmol) was added. The reaction was stirred overnight at room temperature and then diluted with brine. The mixture was extracted with ethyl acetate, concentrated, and chromatographed (12 g silica column, ethyl acetate/hexanes) to provide methyl 5-((2-((S)-2-((tert-butoxycarbonyl)amino)-4-methylpentanamido)ethyl)carbamoyl)-2-(2-(4-fluorophenyl)butanamido)-4-methylthiophene-3-carboxylate (26 mg). This material was dissolved in 4N HCl in dioxane (1 mL) and stirred for 4 hr. The reaction was then diluted with saturated aqueous sodium bicarbonate and extracted with dichloromethane. The extracts were concentrated and chromatographed (4 g silica column, methanol/dichloromethane) to provide a colorless oil. The oil was converted to its hydrochloride salt with 2N hydrogen chloride in diethyl ether, diluted with dioxane, and then lyophilized to provide the hydrochloride salt of the title compound as a white powder (21 mg). MS m/z 535.22 (M+H). ¹H NMR (300 MHz, CDCl₃) δ: 11.41 (s, 1H), 7.84 (br s, 1H), 7.24-7.28 (m, 2H), 6.98-7.17 (m, 2H), 6.69 (br s, 1H), 3.83 (s, 3H), 3.35-3.58 (m, 7H), 2.49 (s, 3H), 1.2-2.3 (m, 6H), 0.82-0.98 (m, 9H).

### Methyl 5-((2-((S)-2-((S)-2-amino-4-methylpentanamido)-4-methylpentanamido)ethyl)carbamoyl)-2-(2-(4-fluorophenyl)butanamido)-4-methylthiophene-3-carboxylate (16)

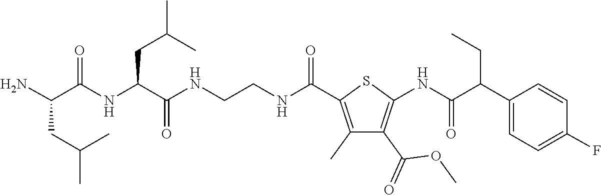 [SMILES: CCC(C(=O)Nc1sc(C(=O)NCCNC(=O)[C@H](CC(C)C)NC(=O)[C@@H](N)CC(C)C)c(C)c1C(=O)OC)c1ccc(F)cc1]

Boc-leucine (8 mg, 0.034 mmol), PyBop (18 mg, 0.034 mmol), and diisopropylethylamine (11 mg, 0.084 mmol) were combined in anhydrous dimethylformamide (1 mL). The mixture was stirred for 15 minutes and then methyl 5-((2-((S)-2-amino-4-methylpentanamido)ethyl)carbamoyl)-2-(2-(4-fluorophenyl)butanamido)-4-methylthiophene-3-carboxylate hydrochloride (16 mg, 0.028 mmol) was added. The reaction was stirred overnight at room temperature and then diluted with brine. The mixture was extracted with ethyl acetate, concentrated, and chromatographed (12 g silica column, ethyl acetate/hexanes) to provide methyl 5-((2-((S)-2-((tert-butoxycarbonyl)amino)-4-methylpentanamido)ethyl)carbamoyl)-2-(2-(4-fluorophenyl)butanamido)-4-methylthiophene-3-carboxylate (17 mg). This material was dissolved in 4N HCl in dioxane (1 mL) and stirred for 3 hr. The reaction was then diluted with saturated aqueous sodium bicarbonate and extracted with dichloromethane. The extracts were concentrated and chromatographed (4 g silica column, dichloromethane/methanol/ammonium hydroxide) to give a colorless oil. The oil was converted to its hydrochloride salt with 4N HCl in dioxane and lyophilized to provide the hydrochloride salt of the title compound as a white powder (17 mg). MS m/z 648.71 (M+H). ¹H NMR (300 MHz, CDCl₃) δ: 11.42 (s, 1H), 7.81 (br s, 1H), 7.31-7.38 (m, 2H), 6.99-7.13 (m, 2H), 6.75 (br s, 1H), 4.30-4.4.0 (m, 1H), 3.83 (s, 3H), 3.16-3.57 (m, 6H), 2.57 (s, 3H), 2.04-2.26 (m, 4H), 1.05-1.90 (m, 7H), 0.80-1.10 (m, 15H).

### Methyl 5-((2-((S)-2-((S)-2-((S)-2-amino-4-methylpentanamido)-4-methylpentanamido)-4-methylpentanamido)ethyl)carbamoyl)-2-(2-(4-fluorophenyl)butanamido)-4-methylthiophene-3-carboxylate (17)

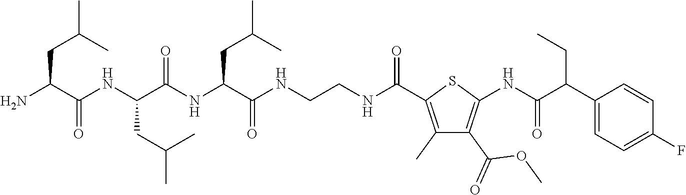 [SMILES: CCC(C(=O)Nc1sc(C(=O)NCCNC(=O)[C@H](CC(C)C)NC(=O)[C@H](CC(C)C)NC(=O)[C@@H](N)CC(C)C)c(C)c1C(=O)OC)c1ccc(F)cc1]

Boc-leucine (7 mg, 0.030 mmol), PyBop (16 mg, 0.030 mmol), and diisopropylethylamine (10 mg, 0.075 mmol) were combined in anhydrous dimethylformamide (1 mL). The mixture was stirred for 15 minutes and then methyl 5-((2-((S)-2-((S)-2-amino-4-methylpentanamido)-4-methylpentanamido)ethyl)carbamoyl)-2-(2-(4-fluorophenyl)butanamido)-4-methylthiophene-3-carboxylate hydrochloride (17 mg, 0.025 mmol) was added. The reaction was stirred overnight at room temperature and then diluted with brine. The mixture was extracted with ethyl acetate, concentrated, and chromatographed (12 g silica column, ethyl acetate/hexanes) to provide methyl 5-(((6S,9S)-6,9-diisobutyl-2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate (13 mg). This material was dissolved in 4N hydrogen chloride in dioxane (1 mL) and stirred overnight. The reaction was then diluted with saturated aqueous sodium bicarbonate and extracted with ethyl acetate. The extracts were concentrated and chromatographed (4 g silica column, methanol/dichloromethane/ammonium hydroxide) to give a colorless oil. The oil was converted to its hydrochloride salt with 4N hydrogen chloride in dioxane and then lyophilized to provide the hydrochloride salt of the title compound as a white powder (11 mg). MS m/z 761.78 (M+H). ¹H NMR (300 MHz, CDCl₃) δ: 11.42 (s, 1H), 7.81-7.88 (m, 1H), 7.28-7.36 (m, 2H), 6.97-7.10 (m, 2H), 6.78-6.82 (m, 1H), 4.17-4.44 (m, 2H), 3.81 (s, 3H), 3.00-3.75 (m, 12H), 2.59 (s, 3H), 0.88-1.97 (m, 30H).

### Methyl 5-((2-((R)-2-amino-3-methylbutanamido)ethyl)carbamoyl)-2-(2-(4-fluorophenyl)butanamido)-4-methylthiophene-3-carboxylate (18)

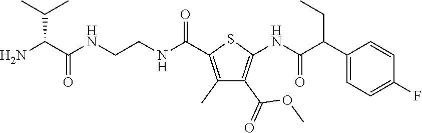 [SMILES: CCC(C(=O)Nc1sc(C(=O)NCCNC(=O)[C@H](N)C(C)C)c(C)c1C(=O)OC)c1ccc(F)cc1]

The hydrochloride salt of the title compound was prepared as described for compound 4, except using Boc-D-valine in place of Boc-L-valine. MS m/z 521.77 (M+H). ¹H NMR (300 MHz, methanol-d₄) δ: 7.53-7.25 (m, 2H), 7.21-7.01 (m, 2H), 3.85 (s, 3H), 3.74 (s, 1H), 3.59 (d, J=5.3 Hz, 1H), 3.57-3.36 (m, 4H), 2.54 (s, 3H), 2.28-2.11 (m, 2H), 2.02-1.74 (m, 1H), 1.03 (t, J=7.6 Hz, 6H), 0.91 (t, J=7.3 Hz, 3H).

### Methyl 5-((2-((R)-2-((R)-2-amino-3-methylbutanamido)-3-methylbutanamido)ethyl)carbamoyl)-2-(2-(4-fluorophenyl)butanamido)-4-methylthiophene-3-carboxylate (19)

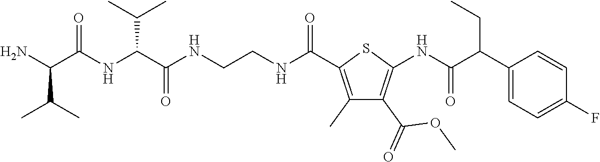 [SMILES: CCC(C(=O)Nc1sc(C(=O)NCCNC(=O)[C@H](NC(=O)[C@H](N)C(C)C)C(C)C)c(C)c1C(=O)OC)c1ccc(F)cc1]

The hydrochloride salt of the title compound was prepared as described for compound 7, except using Boc-D-valine in place of Boc-L-valine. MS m/z 620.78 (M+H). ¹H NMR (300 MHz, methanol-d₄) δ: 7.40 (dd, J=5.6, 8.5 Hz, 2H), 7.11 (t, J=8.8 Hz, 2H), 4.20-4.04 (m, 1H), 3.85 (s, 3H), 3.80-3.60 (m, 4H), 3.51-3.34 (m, 4H), 2.54 (s, 3H), 2.33-1.96 (m, 3H), 1.95-1.77 (m, 1H), 1.05-0.78 (m, 15H).

### Methyl 5-((2-((R)-2-((R)-2-((R)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate (20)

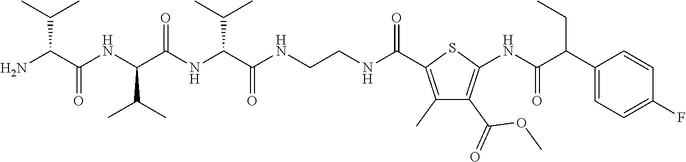 [SMILES: CCC(C(=O)Nc1sc(C(=O)NCCNC(=O)[C@H](NC(=O)[C@H](NC(=O)[C@H](N)C(C)C)C(C)C)C(C)C)c(C)c1C(=O)OC)c1ccc(F)cc1]

The hydrochloride salt of the title compound was prepared as described for compound 9, except using Boc-D-valine in place of Boc-L-valine. MS m/z 719.86 (M+H). ¹H NMR (300 MHz, methanol-d₄) δ: 7.45-7.26 (m, 2H), 7.19-6.94 (m, 2H), 4.25-3.95 (m, 2H), 3.86-3.75 (m, 3H), 3.68 (d, J=6.4 Hz, 2H), 3.35 (d, J=4.1 Hz, 3H), 2.60-2.42 (m, 3H), 2.29-1.67 (m, 5H), 1.05-0.74 (m, 19H).

### Methyl 5-((2-((S)-2-((S)-2-((S)-2-amino-3-phenylpropanamido)-3-methylbutanamido)-3-methylbutanamido)ethyl)carbamoyl)-2-(2-(4-fluorophenyl)butanamido)-4-methylthiophene-3-carboxylate (21)

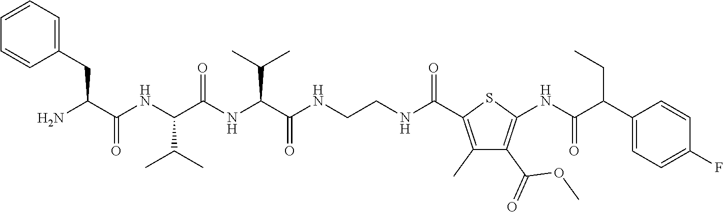 [SMILES: CCC(C(=O)Nc1sc(C(=O)NCCNC(=O)[C@@H](NC(=O)[C@@H](NC(=O)[C@@H](N)Cc2ccccc2)C(C)C)C(C)C)c(C)c1C(=O)OC)c1ccc(F)cc1]

Boc-phenylalanine (13 mg, 0.050 mmol) was suspended in dichloromethane (1 mL). Oxalyl chloride (2 M in dichloromethane, 0.025 mL, 0.063 mol) and dimethylformamide (0.010 mL) were added. The mixture was stirred at room temperature for 1 hr. Methyl 5-((2-((S)-2-((S)-2-amino-3-methylbutanamido)-3-methylbutanamido)ethyl)carbamoyl)-2-(2-(4-fluorophenyl)butanamido)-4-methylthiophene-3-carboxylate (26 mg, 0.042 mmol) was added and the reaction was stirred overnight at room temperature. The reaction was then diluted with saturated aqueous sodium bicarbonate and extracted with dichloromethane. The extracts were concentrated and chromatographed (4 g silica column; hexanes/ethyl acetate) to provide methyl 5-(((6S,9S,12S)-6-benzyl-9,12-diisopropyl-2,2-dimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate as a colorless oil. The oil was dissolved in 4 N hydrogen chloride in dioxane (1 mL) and stirred at room temperature overnight. The solution was lyophilized to provide the hydrochloride salt of the title compound as a white solid (6 mg). MS m/z 768.01 (M+H). ¹H NMR (300 MHz, CDCl₃) δ: 11.43 (s, 1H), 6.9-7.8 (m, 11H), 3.81 (s, 3H), 3.03-3.81 (m, 12H), 2.56 (s, 3H), 0.86-2.03 (m, 21H).

### Methyl 5-((2-((S)-2-((S)-2-(2-aminoacetamido)-3-methylbutanamido)-3-methylbutanamido)ethyl)carbamoyl)-2-(2-(4-fluorophenyl)butanamido)-4-methylthiophene-3-carboxylate (22)

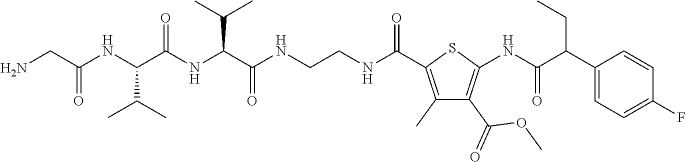 [SMILES: CCC(C(=O)Nc1sc(C(=O)NCCNC(=O)[C@@H](NC(=O)[C@@H](NC(=O)CN)C(C)C)C(C)C)c(C)c1C(=O)OC)c1ccc(F)cc1]

Boc-glycine (11 mg, 0.063 mmol) was dissolved in dichloromethane (1 mL), then oxalyl chloride (2 M in dichloromethane, 0.032 mL, 0.063 mol) and dimethylformamide (0.010 mL) were added. The mixture was stirred at room temperature for 1 hour. Methyl 5-((2-((S)-2-((S)-2-amino-3-methylbutanamido)-3-methylbutanamido)ethyl)carbamoyl)-2-(2-(4-fluorophenyl)butanamido)-4-methylthiophene-3-carboxylate (25 mg, 0.042 mmol) was added and the reaction was stirred overnight at room temperature. The reaction was then diluted with saturated aqueous sodium bicarbonate and extracted with dichloromethane. The extracts were concentrated and chromatographed (4 g silica column; hexanes/ethyl acetate) to provide methyl 5-(((9S,12S)-9,12-diisopropyl-2,2-dimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate as a colorless oil. The oil was dissolved in 4 N hydrogen chloride in dioxane (1 mL) and stirred at room temperature overnight. The solution was lyophilized to provide the hydrochloride salt of the title compound as a white solid (9 mg). MS m/z 677.91 (M+H). ¹H NMR (300 MHz, CDCl₃) δ: 11.40 (s, 1H), 7.80 (br s, 1H), 7.29-7.40 (m, 2H), 7.02-7.21 (m, 3H), 3.83 (s, 3H), 3.03-3.81 (m, 14H), 2.54 (s, 3H), 0.86-2.03 (m, 18H).

### Methyl 5-(((6S,9S)-6,9-diisopropyl-2,2-dimethyl-4,7,10-trioxo-3-oxa-5,8,11-triazatridecan-13-yl)carbamoyl)-4-methyl-2-(2-(4-(trifluoromethyl)phenyl)butanamido)thiophene-3-carboxylate (23)

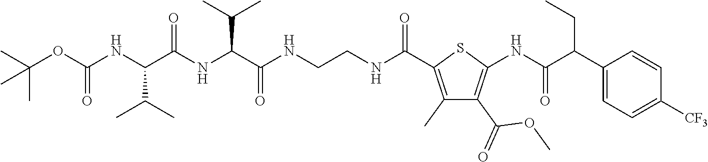 [SMILES: CCC(C(=O)Nc1sc(C(=O)NCCNC(=O)[C@@H](NC(=O)[C@@H](NC(=O)OC(C)(C)C)C(C)C)C(C)C)c(C)c1C(=O)OC)c1ccc(C(F)(F)F)cc1]

Boc-Val-Val-OH (191 mg, 0.603 mmol), PyBOP (314 mg, 0.603 mmol), and diisopropylethylamine (156 mg, 1.21 mmol) were combined in anhydrous dimethylformamide (1 mL). The mixture was stirred for 15 minutes and then methyl 5-(2-aminoethylcarbamoyl)-2-(2-(4-(trifluoromethyl)phenyl)butanamido)-4-methylthiophene-3-carboxylate·HCl (204 mg, 0.402 mmol) was added. The reaction was stirred overnight at room temperature and then diluted with brine. The mixture was extracted with ethyl acetate, concentrated, and chromatographed (12 g silica column, ethyl acetate/hexanes) to provide the title compound as a white solid (240 mg). MS m/z 770.54 (M+H). ¹H NMR (300 MHz, CDCl₃) δ: 11.22 (s, 1H), 7.99 (s, 3H), 7.45 (d, 2H, J=8.8 Hz), 7.42 (d, 2H, J=8.8 Hz), 4.2 (m, 2H), 3.80 (s, 3H), 3.4-3.6 (m 3H), 2.48 (s, 3H), 1.8-2.3 (m, 7H), 1.36-1.40 (m, 9H), 0.8-1.0 (m, 15H).

### Methyl 5-((2-((S)-2-((S)-2-amino-3-methylbutanamido)-3-methylbutanamido)ethyl)carbamoyl)-4-methyl-2-(2-(4-(trifluoromethyl)phenyl)butanamido)thiophene-3-carboxylate (24)

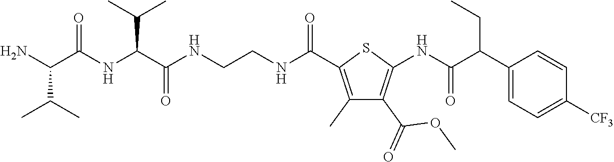 [SMILES: CCC(C(=O)Nc1sc(C(=O)NCCNC(=O)[C@@H](NC(=O)[C@@H](N)C(C)C)C(C)C)c(C)c1C(=O)OC)c1ccc(C(F)(F)F)cc1]

Methyl 5-(((6S,9S)-6,9-diisopropyl-2,2-dimethyl-4,7,10-trioxo-3-oxa-5,8,11-triazatridecan-13-yl)carbamoyl)-4-methyl-2-(2-(4-(trifluoromethyl)phenyl)butanamido)thiophene-3-carboxylate (230 mg, 0.299 mmol) was dissolved in 4 N hydrogen chloride in dioxane (10 mL) and stirred overnight. The solution was lyophilized to provide the hydrochloride salt of the title compound as a white solid (204 mg). MS m/z 670.57 (M+H). ¹H NMR (300 MHz, DMSO-d₆) δ: 11.2 (s, 1H), 7.99 (br s, 1H), 7.65 (d, 2H, J=8.8 Hz), 7.45 (d, 2H, J=8.8 Hz), 3.0-4.0 (m, 21H), 0.8-1.0 (m, 15H).

### Methyl 4-methyl-2-(2-(4-(trifluoromethyl)phenyl)butanamido)-5-(((6S,9S 12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate (25)

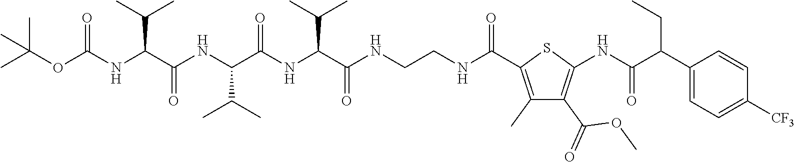 [SMILES: CCC(C(=O)Nc1sc(C(=O)NCCNC(=O)[C@@H](NC(=O)[C@@H](NC(=O)[C@@H](NC(=O)OC(C)(C)C)C(C)C)C(C)C)C(C)C)c(C)c1C(=O)OC)c1ccc(C(F)(F)F)cc1]

Boc-Val-OH (16 mg, 0.074 mmol), PyBOP (30 mg, 0.074 mmol), and diisopropylethylamine (22 mg, 0.17 mmol) were combined in anhydrous dimethylformamide (1 mL). The mixture was stirred for 15 minutes and then methyl 5-((2-((S)-2-((S)-2-amino-3-methylbutanamido)-3-methylbutanamido)ethyl)carbamoyl)-4-methyl-2-(2-(4-(trifluoromethyl)phenyl)butanamido)thiophene-3-carboxylate (40 mg, 0.057 mmol) was added. The reaction was stirred overnight at room temperature and then diluted with brine. The mixture was extracted with ethyl acetate, concentrated, and chromatographed (12 g silica column, ethyl acetate/hexanes) to provide the title compound as a white solid (17 mg). MS m/z 869.68 (M+H). ¹H NMR (300 MHz, CDCl₃) δ: 11.65 (s, 1H), 8.02 (s, 1H), 7.55-7.65 (m, 2H), 7.43-7.55 (m, 2H), 7.0-7.4 (m, 2H), 5.24-5.45 (m, 2H), 4.0-4.5 (4H), 3.78-3.82 (m, 3H), 3.3-3.6 (m, 4H), 2.8-3.0 (m, 6H), 2.45-2.6 (m, 2H), 1.8-2.4 (m, 6H), 1.2-1.5 (m, 10H), 0.75-1.0 (m, 21H).

### Methyl 4-methyl-5-(((6S,9S,12S,15S)-6,9,12,15-tetraisopropyl-2,2-dimethyl-4,7,10,13,16-pentaoxo-3-oxa-5,8,11,14,17-pentaazanonadecan-19-yl)carbamoyl)-2-(2-(4-(trifluoromethyl)phenyl)butanamido)thiophene-3-carboxylate (26)

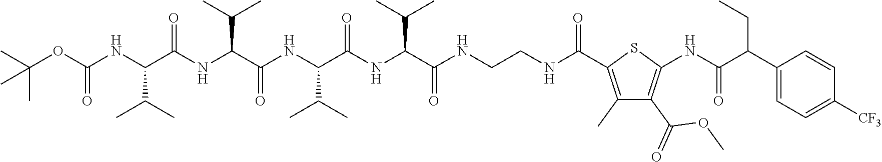 [SMILES: CCC(C(=O)Nc1sc(C(=O)NCCNC(=O)[C@@H](NC(=O)[C@@H](NC(=O)[C@@H](NC(=O)[C@@H](NC(=O)OC(C)(C)C)C(C)C)C(C)C)C(C)C)C(C)C)c(C)c1C(=O)OC)c1ccc(C(F)(F)F)cc1]

Boc-Val-Val-OH (23 mg, 0.074 mmol), PyBOP (30 mg, 0.074 mmol), and diisopropylethylamine (22 mg, 0.17 mmol) were combined in anhydrous dimethylformamide (1 mL). The mixture was stirred for 15 minutes and then methyl 5-((2-((S)-2-((S)-2-amino-3-methylbutanamido)-3-methylbutanamido)ethyl)carbamoyl)-4-methyl-2-(2-(4-(trifluoromethyl)phenyl)butanamido)thiophene-3-carboxylate (40 mg, 0.057 mmol) was added. The reaction was stirred overnight at room temperature and then diluted with brine. The mixture was extracted with ethyl acetate, concentrated, and chromatographed (12 g silica column, ethyl acetate/hexanes) to provide the title compound as a white solid (31 mg). MS m/z 968.81 (M+H). ¹H NMR (300 MHz, CDCl₃) δ: 11.43-11.60 (m, 1H), 8.0-8.2 (m, 2H), 7.70 (d, 2H), 7.50 (d, 2H), 3.9-4.4 (m, 2H), 3.83 (s, 3H), 3.3-3.6 (m, 4H), 2.8-3.0 (m, 10H), 2.45-2.60 (m, 2H), 1.7-2.3 (m, 8H), 1.2-1.5 (m, 10H), 0.75-1.1 (m, 27H).

Compounds 27-93 can be prepared according to the synthetic routes described elsewhere herein and/or methods known to those skilled in the art in view of the teachings provided elsewhere herein.

### Example 2: TriValine-7269-Conjugate (i.e., Compound 9) Targets mD4 and Blocks Infection of the mD4 Surrogate

In certain embodiments, cellular processes such as uptake or metabolic stability can be a useful starting point for optimization of compound 7269, and analogues thereof. As a means of increasing the bioavailability of compound 7269, short peptide-conjugated analogs of compound 7269 were prepared, as described elsewhere herein.

In certain embodiments, valine peptides were conjugated to compound 7269 so as to increase in lipophilicity. Initially, compound 7269 was individually conjugated to mono-, di-, tri- and tetra-valine to amino acids and/or peptides, wherein, in certain embodiments, a linking moiety was incorporated.

When evaluated for their abilities to block infection of the surrogate virus mD4-VV, the mono-, di-, and tetra-valine conjugates of compound 7269 all proved to be cytotoxic.

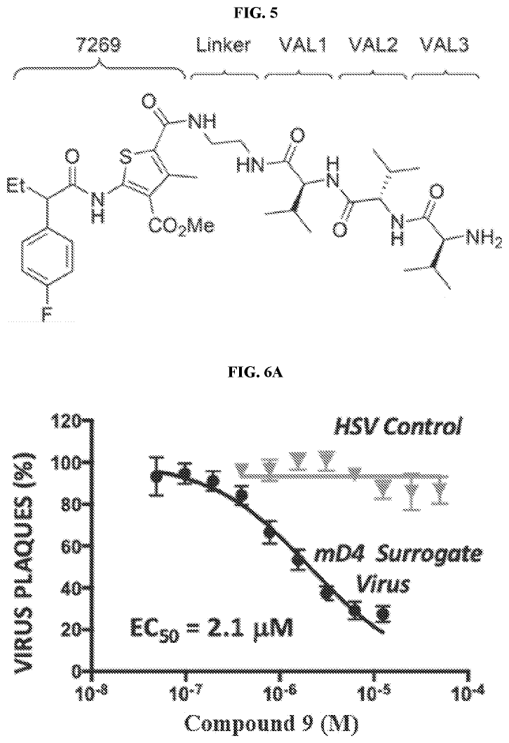

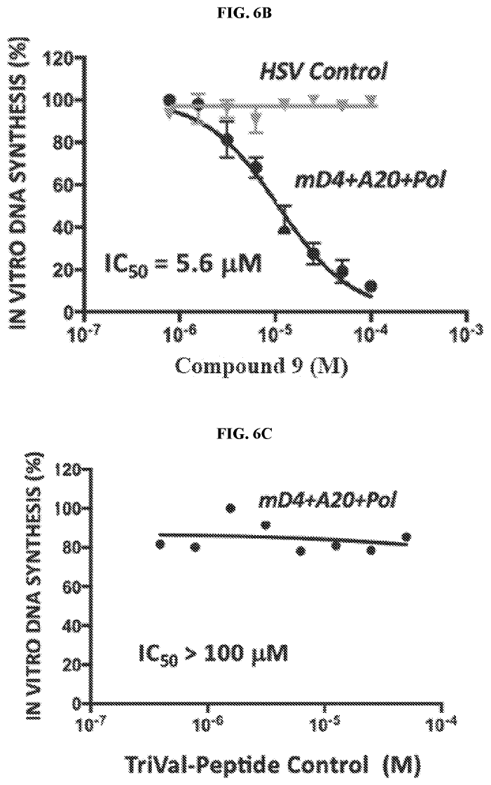

In remarkable contrast, the TriValine-7269 conjugate, compound 9 (FIG. 5) was not only able to block infection of the mD4-VV surrogate virus but did so with a potency (EC₅₀=2.1 μM) that was 6.3-fold greater than that of compound 7269 (FIG. 6A). Compound 9 also specifically targeted mD4 in an in vitro mechanistic assay with an IC₅₀=5.0 μM (FIG. 6B) which was only slightly improved over that of unconjugated compound 7269 (IC₅₀=6.8 μM). The unconjugated TriValine peptide (i.e., ((L)-Val)₃) had no effect (FIG. 6C).

These results indicate that the TriValine moiety does not affect the mechanism (IC₅₀) of the 7269 moiety to directly target mD4. Without wishing to be bound by theory, these results indicate that the increase in antiviral potency (EC₅₀) in the infection assay (FIG. 6A) is derived from the TriValine moiety, supporting the benefit of integrating the individual properties of a peptide with those of a small molecule.

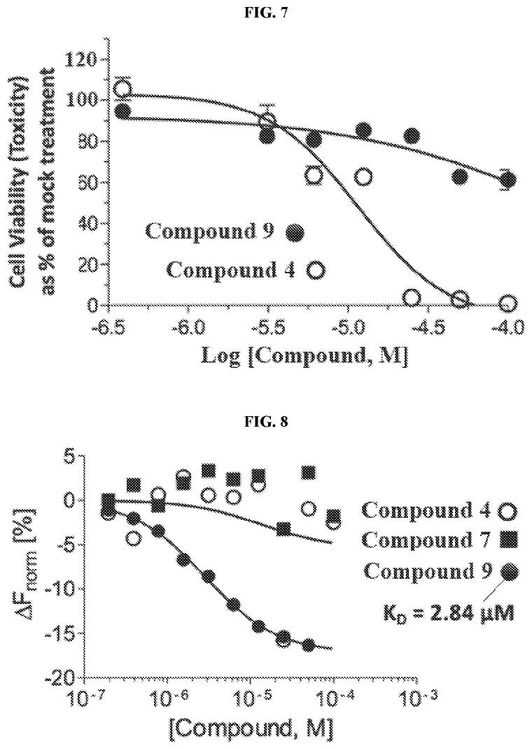

Significantly, compound 9 exhibited no measurable toxicity (CC₅₀>100 μM) when tested in the 3-Day Cell Viability Assay (FIG. 7). This is in contrast to unconjugated compound 7269, which, as described above, was at the verge of toxicity (CC₅₀=103.2 μM). Finally, as confirmed by Micro-Scale Thermophoresis, compound 9 binds to the mD4 viral target (K_{D}=2.84 μM) (FIG. 8).

The present disclosure further provides exemplary mD4 anti-processivity data (Table 2) and/or antiviral mD4/NV activity data (Table 3) for selected compounds.

Table 2 provides 4D4 anti-processivity activity data for exemplary compounds.

Table 3 provides data for exemplary compounds with measurable activity in the antiviral mD4/VV assay.

TABLE 2
mD4 anti-processivity activity for selected compounds

| Compound | IC₅₀ (μM) |
|---|---|
| 5 | 20.0 |
| 8 | 10.7 |
| 9 | 5.0 |
| 20 | 3.0 |
| 26 | 4.1 |

TABLE 3
Antiviral mD4/VV activity for selected compounds

| Cmpd | EC₅₀ (μM) |
|---|---|
| 1 | 29.5 |
| 4 | 22.5 |
| 5 | 16.9 |
| 7 | 23.2 |
| 9 | 2.1 |
| 15 | 28.1 |
| 18 | 14.1 |
| 19 | ~10 |
| 20 | 11.8 |
| 21 | 61.4 |
| 22 | 12.1 |
| 24 | 13.0 |
| 26 | 1.1 |
| 28 | 5.9 |
| 29 | 31.3 |
| 30 | 31.3 |
| 32 | 29.5 |
| 40 | 38.9 |
| 42 | 20 |
| 43 | 29.9 |
| 44 | 35.9 |
| 45 | 33 |
| 48 | 20.5 |
| 49 | 33.4 |
| 53 | 25.8 |
| 54 | 42 |
| 55 | 12.3 |
| 57 | ~25 |
| 58 | 8.7 |
| 60 | 3.5 |
| 61 | 36.1 |
| 69 | 4 |
| 70 | 6 |
| 71 | 4.1 |
| 72 | 7.8 |
| 74 | 1.1 |
| 75 | 2.5 |
| 77 | 9.1 |
| 78 | 2.5 |
| 84 | 26.2 |
| 85 | 20.5 |
| 87 | 30.6 |
| 89 | 12.5 |
| 91 | 2.4 |
| 92 | 35.5 |
| 93 | 13.6 |

### Example 3: Linker and/or Conjugated Amino Acid Modifications

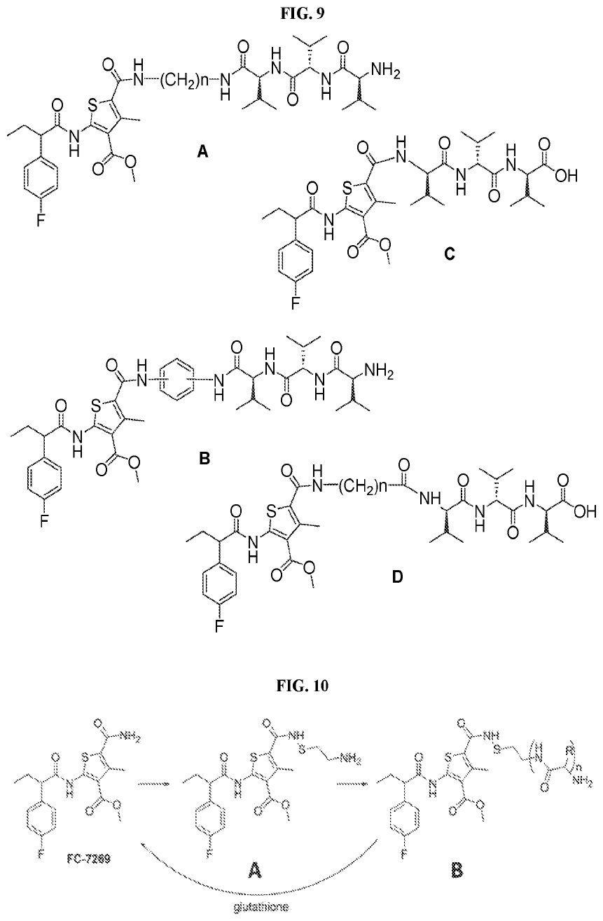

In certain embodiments, the length and geometry of the linker can be varied by using any of a number of alternative divalent species (e.g., —NH(CH₂)₂NH—, —NH(CH₂)₃NH—, —NH(CH₂)₄NH—, —NH(CH₂)₂O(CH₂)₂NH—, 1,4-piperazinyl, 1,2-diaminophenyl, 1,3-diaminophenyl, 1,4-diaminophenyl) (FIG. 9). In certain embodiments, unnatural amino acids (e.g., D-amino acids, substituted L-amino acids, and/or homologated D- and/or L-amino acids) can be utilized. In certain embodiments, unnatural amino acids can permit the use of alternative linking moieties and/or can provide favorable metabolic profiles and/or pharmacokinetics.

For example, cleavage (e.g., by glutathione) of the amino acids conjugated to compound 7269, for example in compound 9, can provide aminoethyl linker substituted 7269, as opposed to compound 7269. Thus, in certain embodiments, the linker can comprise a sulfenamide derivative. In such embodiments, the sulfenamide linker can permit prodrug activity of the compounds of the present disclosure (FIG. 10).

### Enumerated Embodiments

The following exemplary embodiments are provided, the numbering of which is not to be construed as designating levels of importance:

Embodiment 1 provides a compound of formula (I), or a salt, solvate, enantiomer, diastereomer, geometric isomer, isotopologue, or tautomer thereof:

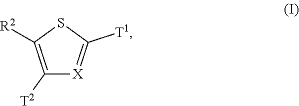

wherein:
T¹ is - - -L²-L¹-R¹ and T² is R³,
   or T¹ is R³ and T² is - - -L²-L¹-R¹;
R¹ is - - -A¹-A²-A³-A⁴;
A¹ is selected from the group consisting of

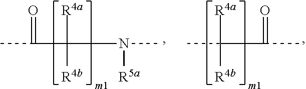

and a bond;
A² is selected from the group consisting of

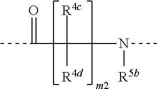

and a bond;
A³ is selected from the group consisting of

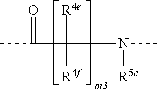

and a bond;
A⁴ is selected from the group consisting of

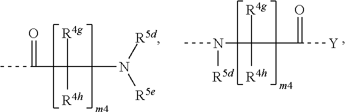

and Y;
L¹ is selected from the group consisting of a bond, —N(R^{a})(optionally substituted C₁-C₆ alkylenyl)N(R^{b})—, —N(R^{a})S (optionally substituted C₁-C₆ alkylenyl)N(R^{b}), —N(R^{a})(optionally substituted C₁-C₆ heteroalkylenyl)N(R^{b}), —N(R^{a})S (optionally substituted C₁-C₆ heteroalkylenyl)N(R^{b})—, —N(R^{a})(optionally substituted C₃-C₈ cycloalkylenyl)N(R^{b})—, —N(R^{a})S (optionally substituted C₃-C₈ cycloalkylenyl)N(R^{b})—, —N(R^{a})(optionally substituted C₂-C₆ heterocyclylenyl)N(R^{b})—, —N(R^{a})S (optionally substituted C₂-C₆ heterocyclylenyl)N(R^{b}), —N(R^{a})(optionally substituted C₁-C₆ alkylenyl)C(═O)—, —N(R^{a})S (optionally substituted C₁-C₆ alkylenyl)C(═O)—, optionally substituted C₁-C₆ alkylenyl, —N(R^{a})(optionally substituted phenylenyl)N(R^{b})—, and —N(R^{a})S (optionally substituted phenylenyl)N(R^{b})—;
L² is selected from the group consisting of —C(═O)—, N(R⁶), and a bond;
Y is selected from the group consisting of N(R^{5e})(R^{5f}), OR^{5e}, and R^{5e},
   wherein A¹, A², A³, A⁴, L¹, L², and Y are selected such that:
      a bond between any substituent selected from the group consisting of A¹, A², A³, and A⁴, and any substituent selected from the group consisting of A¹, A², A³, A⁴, L¹, and L², if present, is a C—N bond, and
      a bond between Y and any substituent selected from the group consisting of A¹, A², A³, and A⁴, if present, is a C—N or C—O bond;
R² is selected from the group consisting of N(R^{a})C(═O) (optionally substituted C₁-C₆ alkyl), N(R^{a})C(═O) (optionally substituted C₃-C₈ cycloalkyl), N(C(═O) (optionally substituted C₃-C₈ cycloalkyl))₂, N(R^{a})C(═O)O (optionally substituted phenyl), N(R^{a})C(═O)(optionally substituted C₂-C₈ heterocyclyl), N(R^{a})C(═O)N(R^{b}) (optionally substituted C₁-C₆ alkyl), C(═O)OR^{a}, and C(═O)N(R^{a})C(═O) (optionally substituted C₁-C₆ alkyl);
R³ is selected from the group consisting of H, optionally substituted C₁-C₆ alkyl, optionally substituted phenyl, CN, NO₂, C(═O)OR^{a}, and C(═O)NR^{a}R^{b};
each occurrence of R^{4a}, R^{4b}, R^{4c}, R^{4d}, R^{4e}, R^{4f}, R^{4g}, and R^{4h} is independently selected from the group consisting of H, optionally substituted C₁-C₆ alkyl, optionally substituted C₃-C₆ cycloalkyl, optionally substituted C₂-C₆ heteroalkyl, optionally substituted C₂-C₆ alkenyl, optionally substituted C₂-C₆ alkynyl, optionally substituted heterocyclyl, and optionally substituted phenyl;
each occurrence of R^{5a}, R^{5b}, R^{5c}, R^{5d}, R^{5e}, and R^{5f} is independently selected from the group consisting of H, optionally substituted C₁-C₆ alkyl, optionally substituted C₃-C₈ cycloalkyl, optionally substituted phenyl, C(═O)R^{a}, and C(═O)OR^{a},
   or two vicinal substituents selected from the group consisting of R^{4a}, R^{4b}, R^{4c}, R^{4d}, R^{4e}, R^{4f}, R^{4h}, R⁴ⁱ, R^{4j}, R^{5a}, R^{5b}, R^{5c}, R^{5d}, R^{5e}, and R^{5f} can optionally combine with the atoms to which they are bound to form an optionally substituted C₄-C₈ heterocycloalkyl;
R⁶ is selected from the group consisting of H and optionally substituted C₁-C₆ alkyl;
X is selected from the group consisting of CR⁶ and N;
m1, m2, m3, and m4 are each independently an integer selected from the group consisting of 1, 2, 3, and 4;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl,
   or geminal R^{a} and R^{b} can optionally combine with the atom to which they are bound to form an optionally substituted C₂-C₈ heterocyclyl.

Embodiment 2 provides the compound of Embodiment 1, which is selected from the group consisting of:

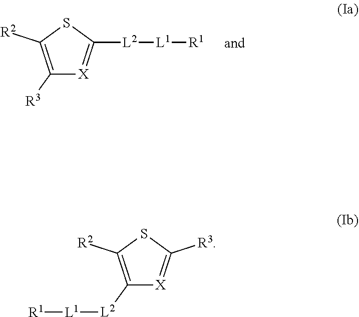

Embodiment 3 provides the compound of Embodiment 1 or 2, wherein each occurrence of optionally substituted alkyl, optionally substituted alkylenyl, optionally substituted cycloalkylenyl, optionally substituted heterocyclylenyl, optionally substituted phenylenyl, optionally substituted cycloalkyl, optionally substituted heteroalkyl, optionally substituted heterocycloalkyl, optionally substituted haloalkyl, optionally substituted alkenyl, and optionally substituted alkynyl is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OR^{I}, N(R^{I})(R^{II}), SR^{I}, C(═O)R^{I}, C(═O)OR^{I}, OC(═O)OR^{I}, C(═O)N(R^{I})(R^{II}), S(═O)₂N(R^{I})(R^{II}), N(R^{I})C(═O)R^{II}, N(R^{I})C(═NR^{II})N(R^{III})(R^{IV}), N(R^{I})S(═O)₂R^{II}, optionally substituted C₂-C₈ heterocyclyl, and optionally substituted phenyl, wherein each occurrence of R^{I}, R^{II}, R^{III}, and R^{IV} is independently selected from the group consisting of H, C₁-C₆ alkyl, C₃-C₈ cycloalkyl, C₁-C₆ haloalkyl, benzyl, and optionally substituted phenyl.

Embodiment 4 provides the compound of any one of Embodiments 1-3, wherein each occurrence of optionally substituted phenyl and optionally substituted heterocyclyl is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, ORⁱ, N(Rⁱ)(Rⁱⁱ), SRⁱ, C(═O)Rⁱ, C(═O)ORⁱ, OC(═O)ORⁱ, C(═O)N(Rⁱ)(Rⁱⁱ), S(═O)₂N(Rⁱ)(Rⁱⁱ), N(Rⁱ)C(═O)Rⁱⁱ, N(Rⁱ)C(═NRⁱⁱ)N(Rⁱⁱⁱ)(R^{iv}), N(Rⁱ)S(═O)₂Rⁱⁱ, C₂-C₈ heterocyclyl, and phenyl, wherein each occurrence of Rⁱ, Rⁱⁱ, Rⁱⁱⁱ, and R^{iv} is independently selected from the group consisting of H, C₁-C₆ alkyl, C₃-C₈ cycloalkyl, C₁-C₆ haloalkyl, benzyl, and phenyl.

Embodiment 5 provides the compound of any one of Embodiments 1-4, wherein one of the following applies:
(a) A⁴ is

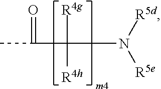

and one of the following applies:
   (i) none of A¹, A², and A³ are a bond,
   (ii) one of A¹, A², and A³ is a bond,
   (iii) two of A¹, A², and A³ are a bond, and
   (iv) each of A¹, A², and A³ are a bond;
(b) A¹ is

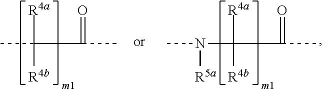

A⁴ is

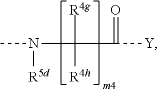

and one of the following applies:
   (i) neither of A² and A³ is a bond,
   (ii) one of A² and A³ is a bond, and
   (iii) both A² and A³ are a bond; or
(c) A¹ is

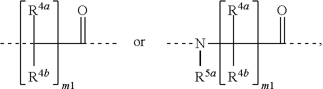

A⁴ is Y, and one of the following applies:
   (i) neither of A² and A³ is a bond,
   (ii) one of A² and A³ is a bond, and
   (iii) both A² and A³ are a bond.

Embodiment 6 provides the compound of any one of Embodiments 1-5, wherein at least one of the following applies:
(a) A¹ is selected from the group consisting of

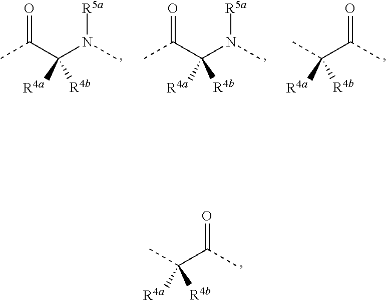

and a bond;
(b) A² is selected from the group consisting of

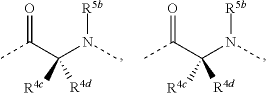

and a bond;
(c) A³ is selected from the group consisting of

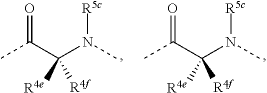

and a bond; and
(d) A⁴ is selected from the group consisting of

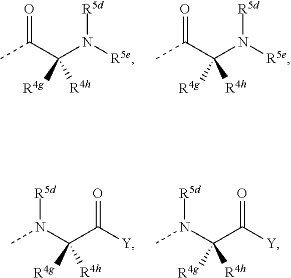

and Y.

Embodiment 7 provides the compound of any one of Embodiments 1-6, wherein R^{4a}, R^{4b}, R^{4c}, R^{4d}, R^{4e}, R^{4f}, R^{4g}, and R^{4h}, if present, are each independently selected from the group consisting of H, methyl,

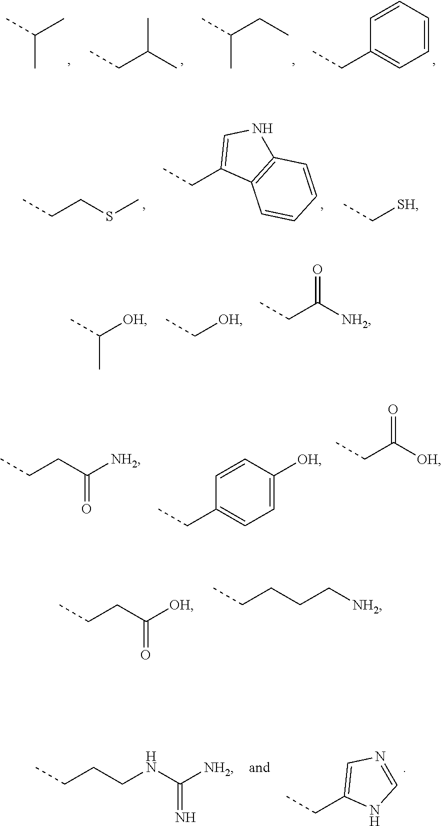

Embodiment 8 provides the compound of any one of Embodiments 1-7, wherein at least one of the following applies:
(a) at least one of R^{4a} and R^{4b} is H;
(b) at least one of R^{4c} and R^{4d} is H;
(c) at least one of R^{4e} and R^{4f} is H; and
(d) at least one of R^{4g} and R^{4h} is H.

Embodiment 9 provides the compound of any one of Embodiments 1-8, wherein at least one of the following applies:
(a) at least one selected from the group consisting of A¹, A², and A³ is selected from the group consisting of

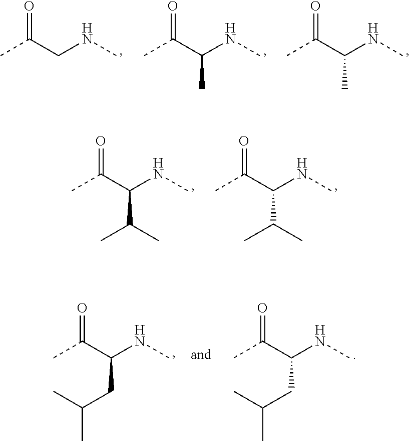

(b) A⁴ is selected from the group consisting of

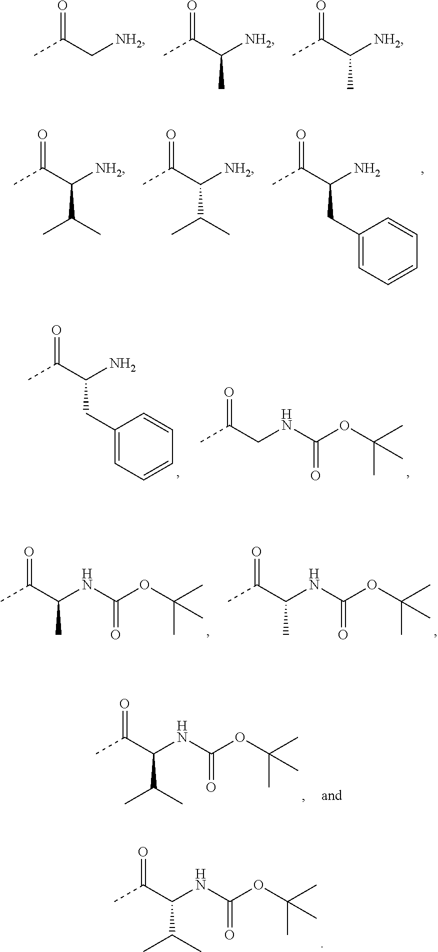

Embodiment 10 provides the compound of any one of Embodiments 1-9, wherein R¹ is selected from the group consisting of NH₂, Ot-Bu, C(═O)Ot-Bu,

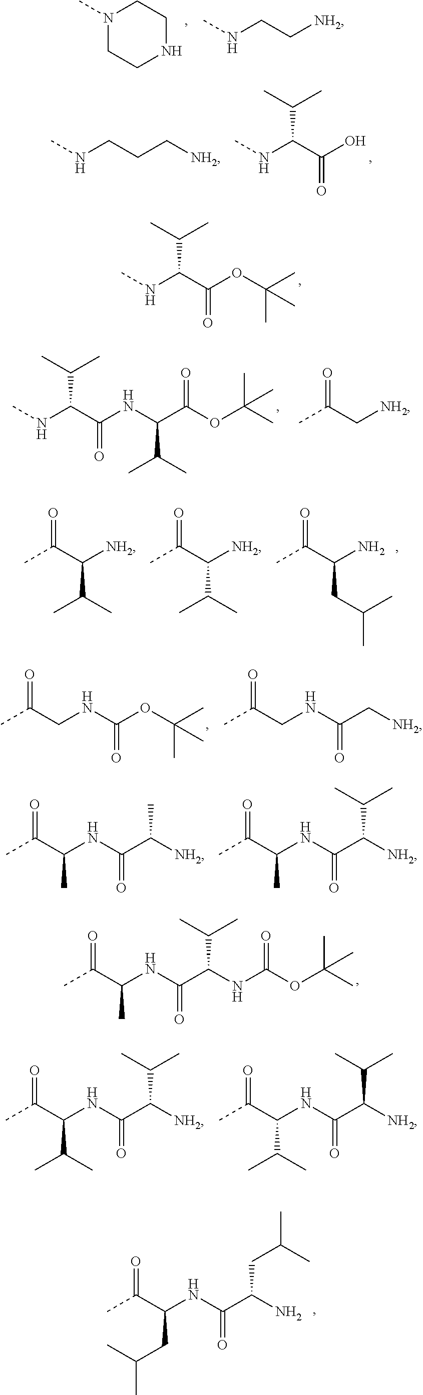

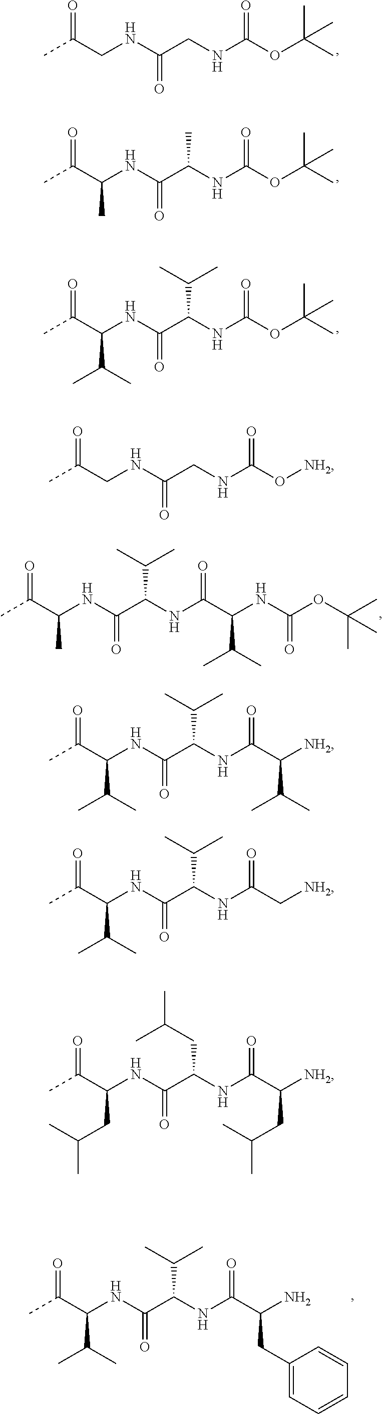

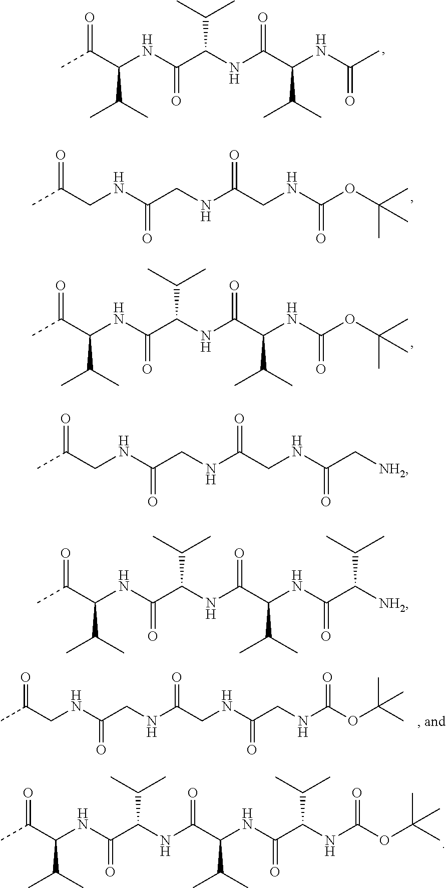

Embodiment 11 provides the compound of any one of Embodiments 1-10, wherein R² is selected from the group consisting of

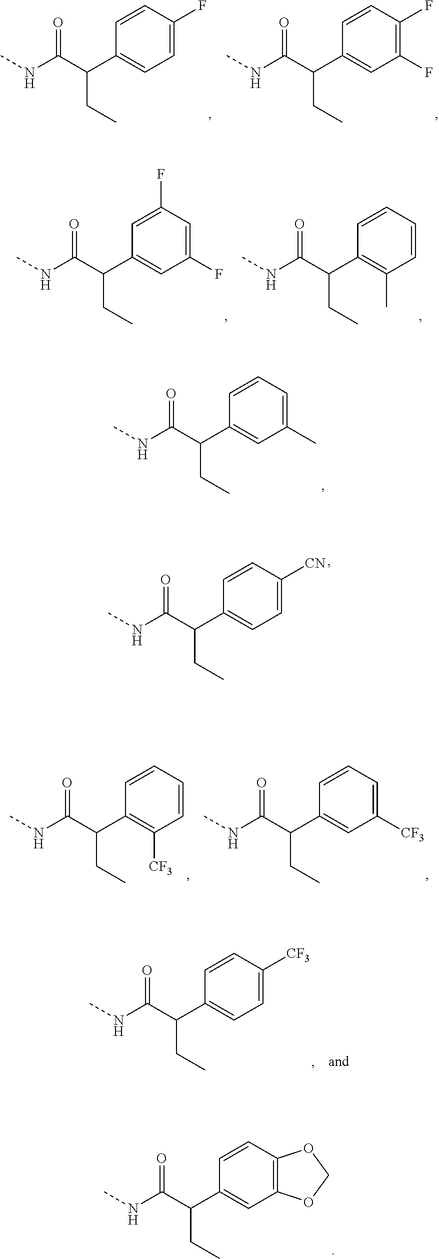

Embodiment 12 provides the compound of any one of Embodiments 1-11, wherein R³ is selected from the group consisting of H, C(═O)OMe, and C(═O)NH₂.

Embodiment 13 provides the compound of any one of Embodiments 1-12, wherein L¹ is selected from the group consisting of

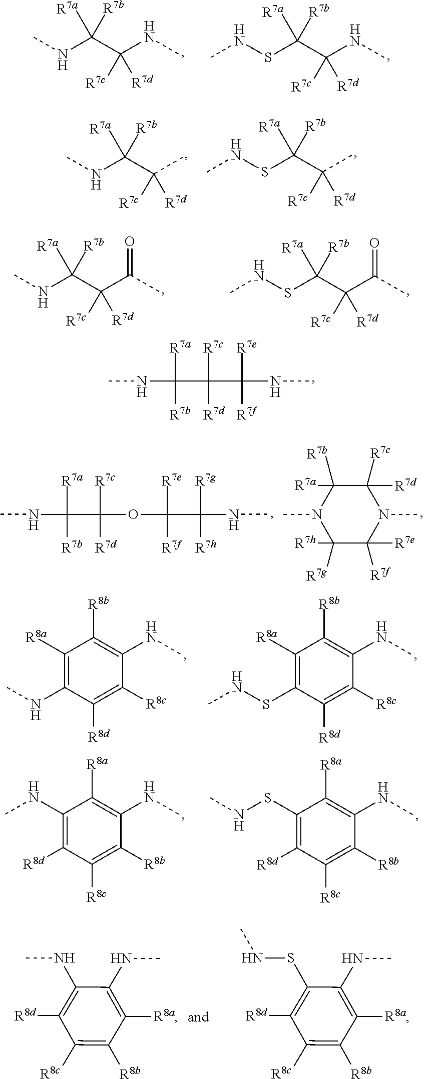

wherein:
R^{7a}, R^{7b}, R^{7c}, R^{7d}, R^{7e}, R^{7f}, R^{7g}, and R^{7h} are each independently selected from the group consisting of H and C₁-C₆ alkyl; and
R^{8a}, R^{8b}, R^{8c}, and R^{8d} are each independently selected from the group consisting of H, C₁-C₆ alkyl, C₁-C₆ alkoxy, C₁-C₃ haloalkyl, C₁-C₆ alkoxy, halogen, CN, and NO₂.

Embodiment 14 provides the compound of Embodiment 13, wherein at least one of the following applies:
(a) at least one of R^{7a}, R^{7b}, R^{7c}, R^{7d}, R^{7e}, R^{7f}, R^{7g}, and R^{7h} is H;
(b) at least two of R^{7a}, R^{7b}, R^{7c}, R^{7d}, R^{7e}, R^{7f}, R^{7g}, and R^{7h} are H;
(c) at least three of R^{7a}, R^{7b}, R^{7c}, R^{7d}, R^{7e}, R^{7f}, R^{7g}, and R^{7h} are H;
(d) at least four of R^{7a}, R^{7b}, R^{7c}, R^{7d}, R^{7e}, R^{7f}, R^{7g}, and R^{7h} are H;
(e) at least five of R^{7a}, R^{7b}, R^{7c}, R^{7d}, R^{7e}, R^{7f}, R^{7g}, and R^{7h} are H;
(f) at least six of R^{7a}, R^{7b}, R^{7c}, R^{7d}, R^{7e}, R^{7f}, R^{7g}, and R^{7h} are H;
(g) at least seven of R^{7a}, R^{7b}, R^{7c}, R^{7d}, R^{7e}, R^{7f}, R^{7g}, and R^{7h} are H;
(h) each of R^{7a}, R^{7b}, R^{7c}, R^{7d}, R^{7e}, R^{7f}, R^{7g}, and R^{7h} are H;
(i) at least one of R^{8a}, R^{8b}, R^{8c}, and R^{8d} is H;
(j) at least two of R^{8a}, R^{8b}, R^{8c}, and R^{8d} are H;
(k) at least three of R^{8a}, R^{8b}, R^{8c}, and R^{8d} are H; and
(l) each of R^{8a}, R^{8b}, R^{8c}, and R^{8d} are H.

Embodiment 15 provides the compound of any one of Embodiments 1-14, wherein L¹ is selected from the group consisting of

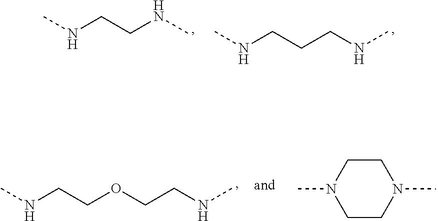

Embodiment 16 provides the compound of any one of Embodiments 1-15, wherein L² is —C(═O)—.

Embodiment 17 provides the compound of any one of Embodiments 1-16, wherein X is C(CH₃).

Embodiment 18 provides the compound of any one of Embodiments 1-17, which is selected from the group consisting of:
methyl 5-((2-(2-((tert-butoxycarbonyl)amino)acetamido)ethyl)carbamoyl)-2-(2-(4-fluorophenyl)butanamido)-4-methylthiophene-3-carboxylate;
methyl 5-((2-(2-aminoacetamido)ethyl)carbamoyl)-2-(2-(4-fluorophenyl)butanamido)-4-methylthiophene-3-carboxylate;
methyl 5-((2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate;
methyl 5-((2-((S)-2-amino-3-methylbutanamido)ethyl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate;
methyl 5-((2-(2-(2-aminoacetamido)acetamido)ethyl)carbamoyl)-2-(2-(4-fluorophenyl)butanamido)-4-methylthiophene-3-carboxylate;
methyl 5-((2-((S)-2-((S)-2-amino-3-methylbutanamido)-3-methylbutanamido)ethyl)carbamoyl)-2-(2-(4-fluorophenyl)butanamido)-4-methylthiophene-3-carboxylate;
methyl 2-(2-(4-fluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate;
methyl 5-(((5S,8S,11S,14S)-14-amino-5,8,11-triisopropyl-15-methyl-4,7,10,13-tetraoxo-3,6,9,12-tetraazahexadecyl)carbamoyl)-2-(2-(4-fluorophenyl)butanamido)-4-methylthiophene-3-carboxylate;
methyl 5-((2,2-dimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate;
methyl 5-((2-(2-(2-(2-aminoacetamido)acetamido)acetamido)ethyl)carbamoyl)-2-(2-(4-fluorophenyl)butanamido)-4-methylthiophene-3-carboxylate;
methyl 5-((2,2-dimethyl-4,7,10,13,16-pentaoxo-3-oxa-5,8,11,14,17-pentaazanonadecan-19-yl)carbamoyl)-2-(2-(4-fluorophenyl)butanamido)-4-methylthiophene-3-carboxylate;
methyl 5-((14-amino-4,7,10,13-tetraoxo-3,6,9,12-tetraazatetradecyl)carbamoyl)-2-(2-(4-fluorophenyl)butanamido)-4-methylthiophene-3-carboxylate;
methyl 5-((2-((S)-2-amino-4-methylpentanamido)ethyl)carbamoyl)-2-(2-(4-fluorophenyl)butanamido)-4-methylthiophene-3-carboxylate;
methyl 5-((2-((S)-2-((S)-2-amino-4-methylpentanamido)-4-methylpentanamido)ethyl)carbamoyl)-2-(2-(4-fluorophenyl)butanamido)-4-methylthiophene-3-carboxylate;
methyl 5-((2-((S)-2-((S)-2-((S)-2-amino-4-methylpentanamido)-4-methylpentanamido)-4-methylpentanamido)ethyl)carbamoyl)-2-(2-(4-fluorophenyl)butanamido)-4-methylthiophene-3-carboxylate;
methyl 5-((2-((R)-2-amino-3-methylbutanamido)ethyl)carbamoyl)-2-(2-(4-fluorophenyl)butanamido)-4-methylthiophene-3-carboxylate;
methyl 5-((2-((R)-2-((R)-2-amino-3-methylbutanamido)-3-methylbutanamido)ethyl)carbamoyl)-2-(2-(4-fluorophenyl)butanamido)-4-methylthiophene-3-carboxylate;
methyl 5-((2-((R)-2-((R)-2-((R)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate;
methyl 5-((2-((S)-2-((S)-2-((S)-2-amino-3-phenylpropanamido)-3-methylbutanamido)-3-methylbutanamido)ethyl)carbamoyl)-2-(2-(4-fluorophenyl)butanamido)-4-methylthiophene-3-carboxylate;
methyl 5-((2-((S)-2-((S)-2-(2-aminoacetamido)-3-methylbutanamido)-3-methylbutanamido)ethyl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3-oxa-5,8,11-triazatridecan-13-yl)carbamoyl)-4-methyl-2-(2-(4-(trifluoromethyl)phenyl)butanamido)thiophene-3-carboxylate;
methyl 5-((2-((S)-2-((S)-2-amino-3-methylbutanamido)-3-methylbutanamido)ethyl)carbamoyl)-4-methyl-2-(2-(4-(trifluoromethyl)phenyl)butanamido)thiophene-3-carboxylate;
methyl 4-methyl-2-(2-(4-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 4-methyl-5-(((6S,9S,12S,15S)-6,9,12,15-tetraisopropyl-2,2-dimethyl-4,7,10,13,16-pentaoxo-3-oxa-5,8,11,14,17-pentaazanonadecan-19-yl)carbamoyl)-2-(2-(4-(trifluoromethyl)phenyl)butanamido)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(4-(trifluoromethyl)phenyl)butanamido)thiophene-3-carboxylate;
methyl 5-(((5S,8S,11S,14S)-14-amino-5,8,11-triisopropyl-15-methyl-4,7,10,13-tetraoxo-3,6,9,12-tetraazahexadecyl)carbamoyl)-4-methyl-2-(2-(4-(trifluoromethyl)phenyl)butanamido)thiophene-3-carboxylate;
methyl 4-methyl-5-(((6S,9S)-2,2,6,9-tetramethyl-4,7,10-trioxo-3-oxa-5,8,11-triazatridecan-13-yl)carbamoyl)-2-(2-(4-(trifluoromethyl)phenyl)butanamido)thiophene-3-carboxylate:
methyl 5-((2-((S)-2-((S)-2-aminopropanamido)propanamido)ethyl)carbamoyl)-4-methyl-2-(2-(4-(trifluoromethyl)phenyl)butanamido)thiophene-3-carboxylate;
methyl 2-(2-(4-fluorophenyl)butanamido)-4-methyl-5-(piperazine-1-carbonyl)thiophene-3-carboxylate;
tert-butyl (2-(5-carbamoyl-2-(2-(4-fluorophenyl)butanamido)-4-methylthiophene-3-carboxamido)ethyl)carbamate;
methyl 5-(4-((tert-butoxycarbonyl)-L-valyl-L-valyl)piperazine-1-carbonyl)-2-(2-(4-fluorophenyl)butanamido)-4-methylthiophene-3-carboxylate;
methyl 5-(4-(L-valyl-L-valyl)piperazine-1-carbonyl)-2-(2-(4-fluorophenyl)butanamido)-4-methylthiophene-3-carboxylate;
N⁴-(2-aminoethyl)-5-(2-(4-fluorophenyl)butanamido)-3-methylthiophene-2,4-dicarboxamide;
tert-butyl ((2S)-1-(((2S)-1-((2-(5-carbamoyl-2-(2-(4-fluorophenyl)butanamido)-4-methylthiophene-3-carboxamido)ethyl)amino)-3-methyl-1-oxobutan-2-yl)amino)-3-methyl-1-oxobutan-2-yl)carbamate;
N⁴-(2-((S)-2-((S)-2-amino-3-methylbutanamido)-3-methylbutanamido)ethyl)-5-(2-(4-fluorophenyl)butanamido)-3-methylthiophene-2,4-dicarboxamide;
methyl 2-(2-(4-fluorophenyl)butanamido)-4-methyl-5-(((5S,8S,11S)-5,8,11-triisopropyl-4,7,10,13-tetraoxo-3,6,9,12-tetraazatetradecyl)carbamoyl)thiophene-3-carboxylate;
methyl 5-(4-(L-valyl-L-valyl-L-valyl)piperazine-1-carbonyl)-2-(2-(4-fluorophenyl)butanamido)-4-methylthiophene-3-carboxylate;
tert-butyl ((7S,10S,13S)-1-(5-carbamoyl-2-(2-(4-fluorophenyl)butanamido)-4-methylthiophen-3-yl)-7,10-diisopropyl-14-methyl-1,6,9,12-tetraoxo-2,5,8,11-tetraazapentadecan-13-yl)carbamate;
methyl 5-((3-((tert-butoxycarbonyl)amino)propyl)carbamoyl)-2-(2-(4-fluorophenyl)butanamido)-4-methylthiophene-3-carboxylate;
methyl 5-((3-aminopropyl)carbamoyl)-2-(2-(4-fluorophenyl)butanamido)-4-methylthiophene-3-carboxylate;
methyl 5-((2-(2-((tert-butoxycarbonyl)amino)ethoxy)ethyl)carbamoyl)-2-(2-(4-fluorophenyl)butanamido)-4-methylthiophene-3-carboxylate;
methyl 2-(2-(4-fluorophenyl)butanamido)-5-(((6S,9S)-6-isopropyl-2,2,9-trimethyl-4,7,10-trioxo-3-oxa-5,8,11-triazatridecan-13-yl)carbamoyl)-4-methylthiophene-3-carboxylate;
methyl 5-((2-((S)-2-((S)-2-amino-3-methylbutanamido)propanamido)ethyl)carbamoyl)-2-(2-(4-fluorophenyl)butanamido)-4-methylthiophene-3-carboxylate;
methyl 5-(((6S,9S,12S)-6,9-diisopropyl-2,2,12-trimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3,14-dioxa-5,8,11-triazahexadecan-16-yl)carbamoyl)-2-(2-(4-fluorophenyl)butanamido)-4-methylthiophene-3-carboxylate;
methyl 5-((3-((S)-2-((S)-2-amino-3-methylbutanamido)-3-methylbutanamido)propyl)carbamoyl)-2-(2-(4-fluorophenyl)butanamido)-4-methylthiophene-3-carboxylate;
methyl 5-((2-(2-((S)-2-((S)-2-amino-3-methylbutanamido)-3-methylbutanamido)ethoxy)ethyl)carbamoyl)-2-(2-(4-fluorophenyl)butanamido)-4-methylthiophene-3-carboxylate;
methyl 2-(2-(benzo[d][1,3]dioxol-5-yl)butanamido)-5-carbamoyl-4-methylthiophene-3-carboxylate;
2-(tert-butyl) 4-methyl 5-(2-(benzo[d][1,3]dioxol-5-yl)butanamido)-3-methylthiophene-2,4-dicarboxylate;
2-(tert-butyl) 4-methyl 5-(2-(3,5-difluorophenyl)butanamido)-3-methylthiophene-2,4-dicarboxylate;
methyl 5-carbamoyl-2-(2-(3,5-difluorophenyl)butanamido)-4-methylthiophene-3-carboxylate;
methyl 5-carbamoyl-2-(2-(4-cyanophenyl)butanamido)-4-methylthiophene-3-carboxylate;
methyl 5-((2-aminoethyl)carbamoyl)-2-(2-(benzo[d][1,3]dioxol-5-yl)butanamido)-4-methylthiophene-3-carboxylate;
methyl 2-(2-(benzo[d][1,3]dioxol-5-yl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(benzo[d][1,3]dioxol-5-yl)butanamido)-4-methylthiophene-3-carboxylate;
methyl 5-((2-aminoethyl)carbamoyl)-2-(2-(3,5-difluorophenyl)butanamido)-4-methylthiophene-3-carboxylate;
methyl 2-(2-(3,5-difluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(3,5-difluorophenyl)butanamido)-4-methylthiophene-3-carboxylate;
methyl 5-((2-aminoethyl)carbamoyl)-2-(2-(4-cyanophenyl)butanamido)-4-methylthiophene-3-carboxylate;
methyl 2-(2-(4-cyanophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(4-cyanophenyl)butanamido)-4-methylthiophene-3-carboxylate;
methyl 2-(2-(4-fluorophenyl)butanamido)-4-methyl-5-(((6S,9S,12S)-6,9,12-triisopropyl-2,2-dimethyl-4,7,10,13-tetraoxo-3,17-dioxa-5,8,11,14-tetraazanonadecan-19-yl)carbamoyl)thiophene-3-carboxylate;
methyl 5-(((8S,11S,14S)-14-amino-8,11-diisopropyl-15-methyl-7,10,13-trioxo-3-oxa-6,9,12-triazahexadecyl)carbamoyl)-2-(2-(4-fluorophenyl)butanamido)-4-methylthiophene-3-carboxylate;
2-(tert-butyl) 4-methyl 5-(2-(3,4-difluorophenyl)butanamido)-3-methylthiophene-2,4-dicarboxylate;
2-(tert-butyl) 4-methyl 3-methyl-5-(2-(m-tolyl)butanamido)thiophene-2,4-dicarboxylate;
2-(tert-butyl) 4-methyl 3-methyl-5-(2-(3-(trifluoromethyl)phenyl)butanamido)thiophene-2,4-dicarboxylate;
methyl 5-carbamoyl-2-(2-(3,4-difluorophenyl)butanamido)-4-methylthiophene-3-carboxylate;
methyl 5-carbamoyl-4-methyl-2-(2-(m-tolyl)butanamido)thiophene-3-carboxylate;
methyl 5-carbamoyl-4-methyl-2-(2-(3-(trifluoromethyl)phenyl)butanamido)thiophene-3-carboxylate;
methyl 5-((2-aminoethyl)carbamoyl)-2-(2-(3,4-difluorophenyl)butanamido)-4-methylthiophene-3-carboxylate:
methyl 2-(2-(3,4-difluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(3,4-difluorophenyl)butanamido)-4-methylthiophene-3-carboxylate;
methyl 5-((2-aminoethyl)carbamoyl)-4-methyl-2-(2-(m-tolyl)butanamido)thiophene-3-carboxylate;
methyl 4-methyl-2-(2-(m-tolyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(m-tolyl)butanamido)thiophene-3-carboxylate;
methyl 5-((2-aminoethyl)carbamoyl)-4-methyl-2-(2-(3-(trifluoromethyl)phenyl)butanamido)thiophene-3-carboxylate;
methyl 4-methyl-2-(2-(3-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(3-(trifluoromethyl)phenyl)butanamido)thiophene-3-carboxylate;
methyl 5-(((R)-1-(tert-butoxy)-3-methyl-1-oxobutan-2-yl)carbamoyl)-2-(2-(4-fluorophenyl)butanamido)-4-methylthiophene-3-carboxylate;
(5-(2-(4-fluorophenyl)butanamido)-4-(methoxycarbonyl)-3-methylthiophene-2-carbonyl)-D-valine;
2-(tert-butyl) 4-methyl 3-methyl-5-(2-(o-tolyl)butanamido)thiophene-2,4-dicarboxylate;
methyl 5-carbamoyl-4-methyl-2-(2-(o-tolyl)butanamido)thiophene-3-carboxylate;
methyl 5-((2-aminoethyl)carbamoyl)-4-methyl-2-(2-(o-tolyl)butanamido)thiophene-3-carboxylate;
methyl 4-methyl-2-(2-(o-tolyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(o-tolyl)butanamido)thiophene-3-carboxylate;
2-(tert-butyl) 4-methyl 3-methyl-5-(2-(2-(trifluoromethyl)phenyl)butanamido)thiophene-2,4-dicarboxylate;
methyl 5-carbamoyl-4-methyl-2-(2-(2-(trifluoromethyl)phenyl)butanamido)thiophene-3-carboxylate;
methyl 4-methyl-2-(2-(2-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(2-(trifluoromethyl)phenyl)butanamido)thiophene-3-carboxylate;
methyl 5-(((R)-1-(((R)-1-(tert-butoxy)-3-methyl-1-oxobutan-2-yl)amino)-3-methyl-1-oxobutan-2-yl)carbamoyl)-2-(2-(4-fluorophenyl)butanamido)-4-methylthiophene-3-carboxylate; and
(5-(2-(4-fluorophenyl)butanamido)-4-(methoxycarbonyl)-3-methylthiophene-2-carbonyl)-D-valyl-D-valine.

Embodiment 19 provides a pharmaceutical composition comprising at least one compound of any one of Embodiments 1-18 and at least one pharmaceutically acceptable excipient.

Embodiment 20 provides a method of treating, ameliorating, and/or preventing an orthopoxvirus infection in a subject in need thereof, the method comprising administering to the subject a therapeutically effective amount of the compound of any one of Embodiments 1-18 and/or the pharmaceutical composition of Embodiment 19.

Embodiment 21 provides the method of Embodiment 20, wherein the orthopoxvirus is selected from the group consisting of Molluscum contagiosum virus (MCV), camelpox virus, cowpox virus, mousepox virus, horsepox virus, monkeypox virus, raccoonpox virus, tanapox virus, variola (smallpox) virus, Yoka poxvirus, cervidpoxvirus (deerpox), avipoxvirus (fowlpox), capripoxvirus (goatpox), leporipoxvirus (myxoma virus), parapoxvirus (orf virus), suipoxvirus (swinepox), and yatapoxvirus (Yaba-like disease virus).

Embodiment 22 provides the method of Embodiment 21, wherein folding and/or function of processivity factor mD4 is inhibited in the orthopoxvirus.

Embodiment 23 provides the method of any one of Embodiments 20-22, wherein DNA polymerase processivity is disrupted in the orthopoxvirus.

Embodiment 24 provides the method of any one of Embodiments 20-23, wherein the orthopoxvirus infection is caused by a MCV.

Embodiment 25 provides the method of any one of Embodiments 20-24, wherein the subject is a mammal.

Embodiment 26 provides the method of Embodiment 25, wherein the mammal is a human.

Embodiment 27 provides the method of any one of Embodiments 20-26, wherein the compound of any one of Embodiments 1-18 and/or the pharmaceutical composition of Embodiment 19 is administered topically.

Embodiment 28 provides the method of any one of Embodiments 20-27, wherein the compound of any one of Embodiments 1-18 and/or the pharmaceutical composition of Embodiment 19 is administered topically to at least one lesion associated with the orthopoxvirus infection.

The disclosures of each and every patent, patent application, and publication cited herein are hereby incorporated herein by reference in their entirety. While this disclosure has been disclosed with reference to specific embodiments, it is apparent that other embodiments and variations of this disclosure can be devised by others skilled in the art without departing from the true spirit and scope of the disclosure. The appended claims are intended to be construed to include all such embodiments and equivalent variations.

1. A compound of formula (I), or a salt, solvate, enantiomer, diastereomer, geometric isomer, isotopologue, or tautomer thereof: 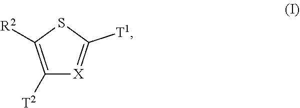
wherein:
T¹ is - - -L²-L¹-R¹ and T² is R³,
or T¹ is R³ and T² is - - -L²-L¹-R¹;
R¹ is - - -A¹-A²-A³-A⁴;
A¹ is selected from the group consisting of 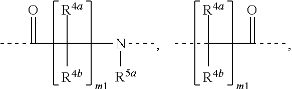
and a bond;
A² is selected from the group consisting of 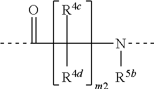
and a bond;
A³ is selected from the group consisting of 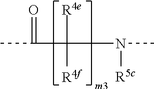
and a bond;
A⁴ is selected from the group consisting of 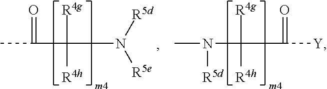
and Y;
L¹ is selected from the group consisting of a bond, —N(R^{a}) (optionally substituted C₁-C₆ alkylenyl)N(R^{b})—, —N(R^{a})S (optionally substituted C₁-C₆ alkylenyl)N(R^{b})—, —N(R^{a}) (optionally substituted C₁-C₆ heteroalkylenyl)N(R^{b}), —N(R^{a})S (optionally substituted C₁-C₆ heteroalkylenyl)N(R^{b})—, —N(R^{a}) (optionally substituted C₃-C₈ cycloalkylenyl)N(R^{b})—, —N(R^{a})S (optionally substituted C₃-C₈ cycloalkylenyl)N(R^{b})—, —N(R^{a}) (optionally substituted C₂-C₆ heterocyclylenyl)N(R^{b})—, —N(R^{a})S (optionally substituted C₂-C₆ heterocyclylenyl)N(R^{b})—, —N(R^{a}) (optionally substituted C₁-C₆ alkylenyl)C(═O)—, —N(R^{a})S (optionally substituted C₁-C₆ alkylenyl)C(═O)—, optionally substituted C₁-C₆ alkylenyl, —N(R^{a}) (optionally substituted phenylenyl)N(R^{b})—, and —N(R^{a})S (optionally substituted phenylenyl)N(R^{b})—;
L² is selected from the group consisting of —C(═O)—, N(R⁶), and a bond;
Y is selected from the group consisting of N(R^{5e})(R^{5f}), OR^{5e}, and R^{5e},
wherein A¹, A², A³, A⁴, L¹, L², and Y are selected such that:
a bond between any substituent selected from the group consisting of A¹, A², A³, and A⁴, and any substituent selected from the group consisting of A¹, A², A³, A⁴, L¹, and L², if present, is a C—N bond, and
a bond between Y and any substituent selected from the group consisting of A¹, A², A³, and A⁴, if present, is a C—N or C—O bond;
R² is selected from the group consisting of N(R^{a})C(═O) (optionally substituted C₁-C₆ alkyl), N(R^{a})C(═O) (optionally substituted C₃-C₈ cycloalkyl), N(C(═O) (optionally substituted C₃-C₈ cycloalkyl))₂, N(R^{a})C(═O)O (optionally substituted phenyl), N(R^{a})C(═O) (optionally substituted C₂-C₈ heterocyclyl), N(R^{a})C(═O)N(R^{b}) (optionally substituted C₁-C₆ alkyl), C(═O)OR^{a}, and C(═O)N(R^{a})C(═O) (optionally substituted C₁-C₆ alkyl);
R³ is selected from the group consisting of H, optionally substituted C₁-C₆ alkyl, optionally substituted phenyl, CN, NO₂, C(═O)OR^{a}, and C(═O)NR^{a}R^{b};
each occurrence of R^{4a}, R^{4b}, R^{4c}, R^{4d}, R^{4e}, R^{4f}, R^{4g}, and R^{4h} is independently selected from the group consisting of H, optionally substituted C₁-C₆ alkyl, optionally substituted C₃-C₆ cycloalkyl, optionally substituted C₂-C₆ heteroalkyl, optionally substituted C₂-C₆ alkenyl, optionally substituted C₂-C₆ alkynyl, optionally substituted heterocyclyl, and optionally substituted phenyl;
each occurrence of R^{5a}, R^{5b}, R^{5c}, R^{5d}, R^{5e}, and R^{5f} is independently selected from the group consisting of H, optionally substituted C₁-C₆ alkyl, optionally substituted C₃-C₈ cycloalkyl, optionally substituted phenyl, C(═O)R^{a}, and C(═O)OR^{a},
or two vicinal substituents selected from the group consisting of R^{4a}, R^{4b}, R^{4c}, R^{4d}, R^{4e}, R^{4f}, R^{4g}, R^{4h}, R⁴ⁱ, R^{4j}, R^{5a}, R^{5b}, R^{5c}, R^{5d}, R^{5e}, and R^{5f} can optionally combine with the atoms to which they are bound to form an optionally substituted C₄-C₈ heterocycloalkyl;
R⁶ is selected from the group consisting of H and optionally substituted C₁-C₆ alkyl;
X is selected from the group consisting of CR⁶ and N;
m1, m2, m3, and m4 are each independently an integer selected from the group consisting of 1, 2, 3, and 4;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted benzyl, optionally substituted phenyl, and optionally substituted C₂-C₈ heterocyclyl,
or geminal R^{a} and R^{b} can optionally combine with the atom to which they are bound to form an optionally substituted C₂-C₈ heterocyclyl.

2. The compound of claim 1, which is selected from the group consisting of:

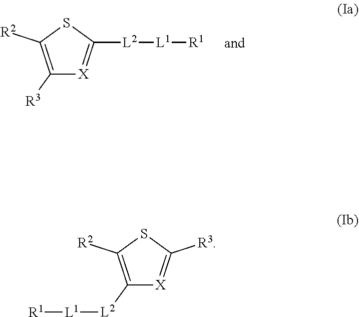

3. The compound of claim 1, wherein each occurrence of optionally substituted alkyl, optionally substituted alkylenyl, optionally substituted cycloalkylenyl, optionally substituted heterocyclylenyl, optionally substituted phenylenyl, optionally substituted cycloalkyl, optionally substituted heteroalkyl, optionally substituted heterocycloalkyl, optionally substituted haloalkyl, optionally substituted alkenyl, and optionally substituted alkynyl is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OR^{I}, N(R^{I})(R^{II}), SR^{I}, C(═O)R^{I}, C(═O)OR^{I}, OC(═O)OR^{I}, C(═O)N(R^{I})(R^{II}), S(═O)₂N(R^{I})(R^{II}), N(R^{I})C(═O)R^{II}, N(R^{I})C(═NR^{II})N(R^{III})(R^{IV}), N(R^{I})S(═O)₂R^{II}, optionally substituted C₂-C₈ heterocyclyl, and optionally substituted phenyl, wherein each occurrence of R^{I}, R^{II}, R^{III}, and R^{IV} is independently selected from the group consisting of H, C₁-C₆ alkyl, C₃-C₈ cycloalkyl, C₁-C₆ haloalkyl, benzyl, and optionally substituted phenyl.

4. The compound of claim 1, wherein each occurrence of optionally substituted phenyl and optionally substituted heterocyclyl is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, ORⁱ, N(Rⁱ)(Rⁱⁱ), SRⁱ, C(═O)Rⁱ, C(═O)ORⁱ, OC(═O)ORⁱ, C(═O)N(Rⁱ)(Rⁱⁱ), S(═O)₂N(Rⁱ)(Rⁱⁱ), N(Rⁱ)C(═O)Rⁱⁱ, N(Rⁱ)C(═NRⁱⁱ)N(Rⁱⁱⁱ)(R^{iv}), N(Rⁱ)S(═O)₂Rⁱⁱ, C₂-C₈ heterocyclyl, and phenyl, wherein each occurrence of Rⁱ, Rⁱⁱ, Rⁱⁱⁱ, and R^{iv} is independently selected from the group consisting of H, C₁-C₆ alkyl, C₃-C₈ cycloalkyl, C₁-C₆ haloalkyl, benzyl, and phenyl.

5. The compound of claim 1, wherein one of the following applies:
(a) A⁴ is 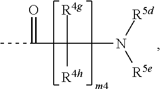
and one of the following applies:
(i) none of A¹, A², and A³ are a bond,
(ii) one of A¹, A², and A³ is a bond,
(iii) two of A¹, A², and A³ are a bond, and
(iv) each of A¹, A², and A³ are a bond;
(b) A¹ is 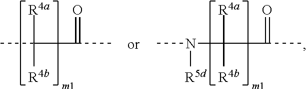
A⁴ is 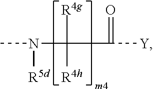
and one of the following applies:
(i) neither of A² and A³ is a bond,
(ii) one of A² and A³ is a bond, and
(iii) both A² and A³ are a bond; or
(c) A¹ is 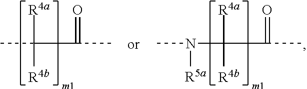
A⁴ is Y, and one of the following applies:
(i) neither of A² and A³ is a bond,
(ii) one of A² and A³ is a bond, and
(iii) both A² and A³ are a bond.

6. The compound of claim 1, wherein at least one of the following applies:
(a) A¹ is selected from the group consisting of 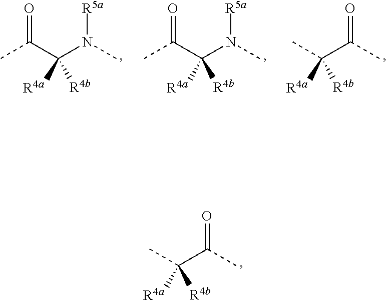
and a bond;
(b) A² is selected from the group consisting of 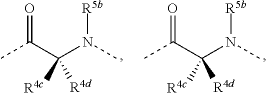
and a bond;
(c) A³ is selected from the group consisting of 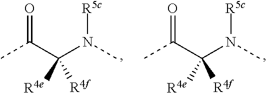
and a bond; and
(d) A⁴ is selected from the group consisting of 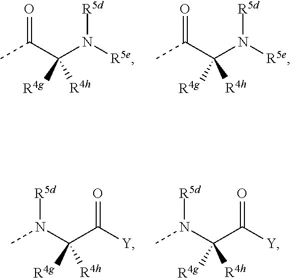
and Y.

7. The compound of claim 1, wherein R^{4a}, R^{4b}, R^{4c}, R^{4d}, R^{4e}, R^{4f}, R^{4g}, and R^{4h}, if present, are each independently selected from the group consisting of H, methyl,

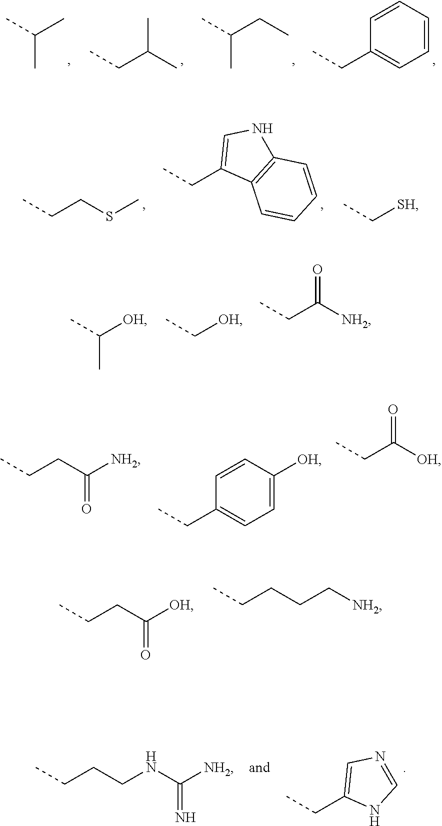

8. The compound of claim 1, wherein at least one of the following applies:
(a) at least one of R^{4a} and R^{4b} is H;
(b) at least one of R^{4c} and R^{4d} is H;
(c) at least one of R^{4e} and R^{4f} is H; and
(d) at least one of R^{4g} and R^{4h} is H.

9. The compound of claim 1, wherein at least one of the following applies:
(a) at least one selected from the group consisting of A¹, A², and A³ is selected from the group consisting of 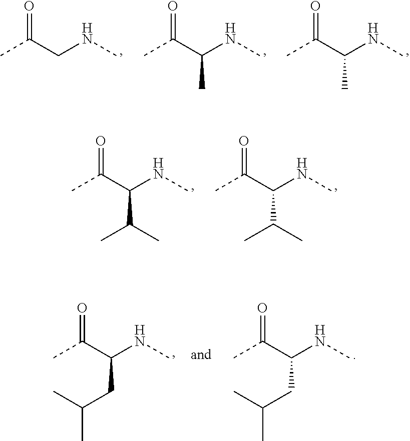
(b) A⁴ is selected from the group consisting of

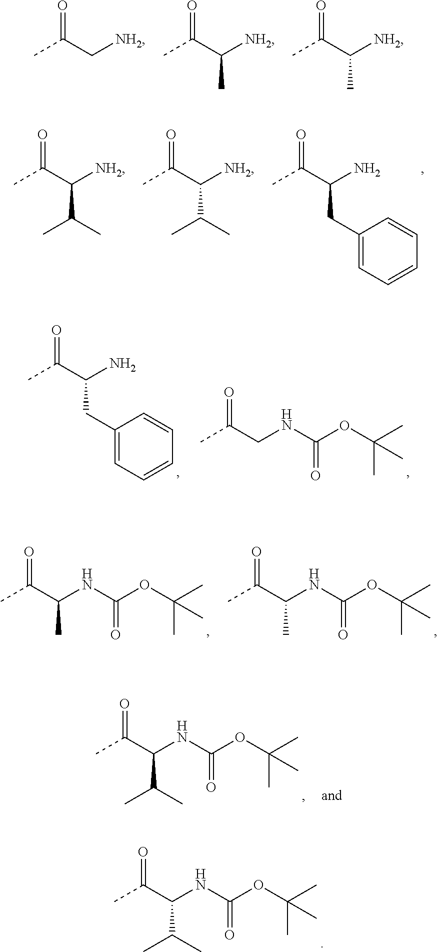

10. The compound of claim 1, wherein at least one of the following applies:
(a) R¹ is selected from the group consisting of NH₂, Ot-Bu, C(═O)Ot-Bu, 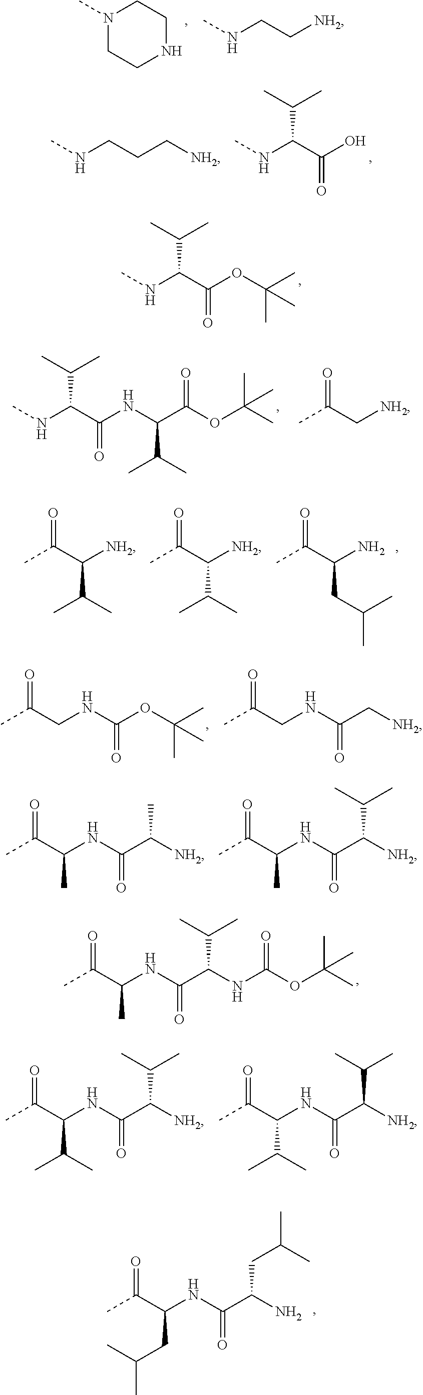 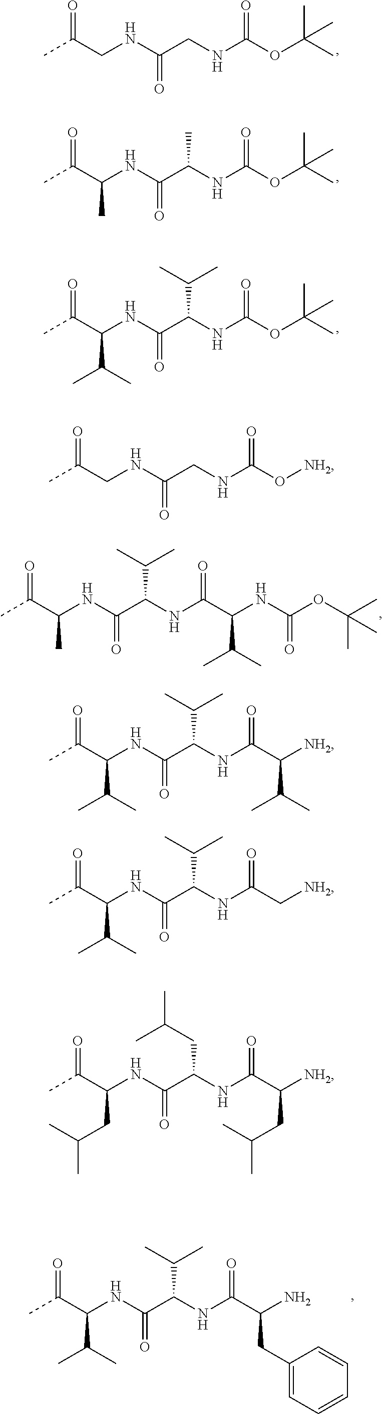 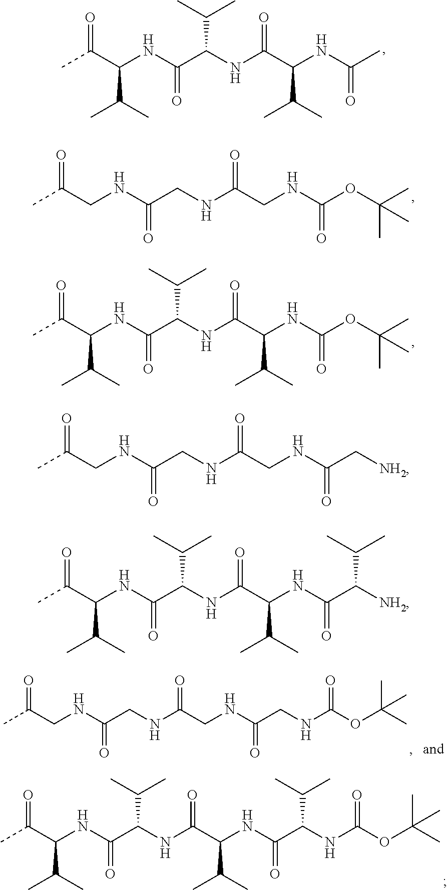
(b) R² is selected from the group consisting of 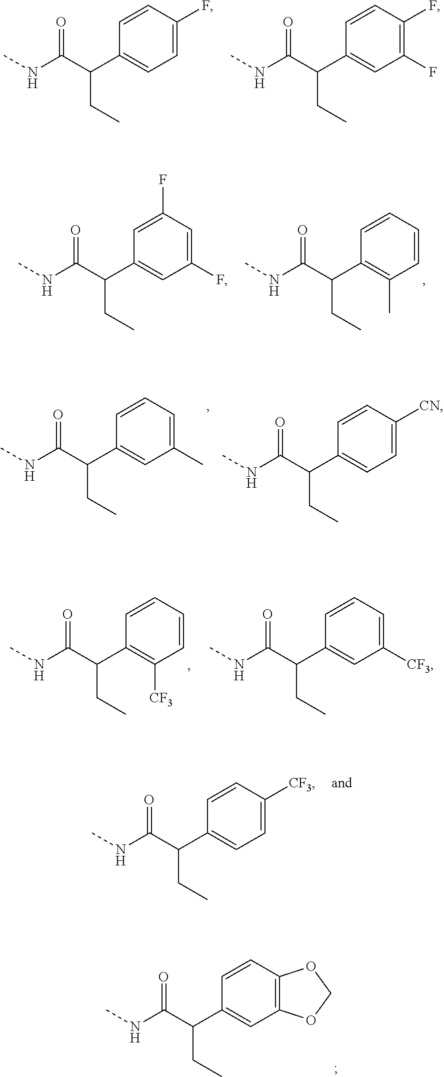
and
(c) R³ is selected from the group consisting of H, C(═O)OMe, and C(═O)NH₂.

11. (canceled)

12. (canceled)

13. The compound of claim 1, wherein L¹ is selected from the group consisting of 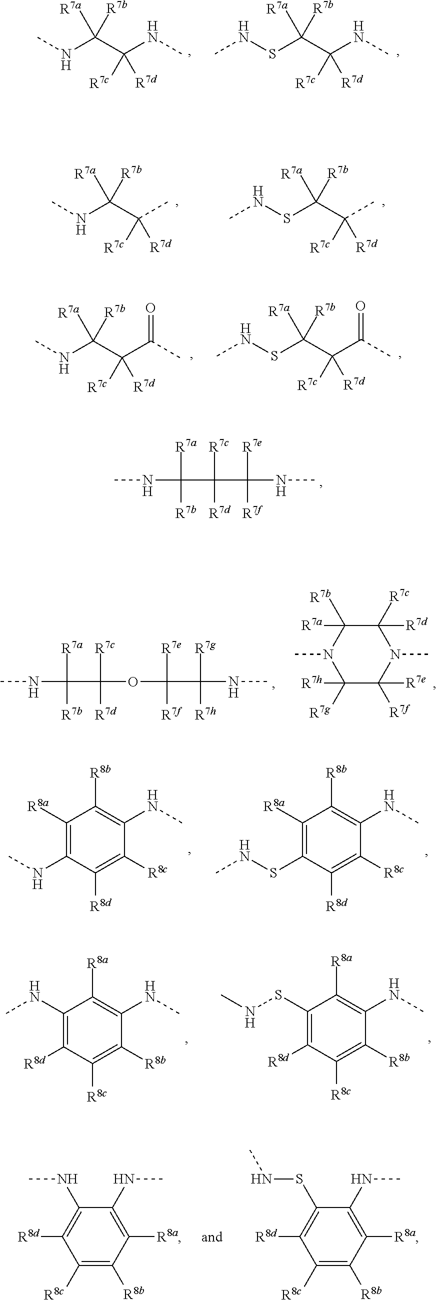
wherein:
R^{7a}, R^{7b}, R^{7c}, R^{7d}, R^{7e}, R^{7f}, R^{7g}, and R^{7h} are each independently selected from the group consisting of H and C₁-C₆ alkyl; and
R^{8a}, R^{8b}, R^{8c}, and R^{8d} are each independently selected from the group consisting of H, C₁-C₆ alkyl, C₁-C₆ alkoxy, C₁-C₃ haloalkyl, C₁-C₆ alkoxy, halogen, CN, and NO₂.

14. The compound of claim 13, wherein at least one of the following applies:
(a) at least one of R^{7a}, R^{7b}, R^{7c}, R^{7d}, R^{7e}, R^{7f}, R^{7g}, and R^{7h} is H;
(b) at least two of R^{7a}, R^{7b}, R^{7c}, R^{7d}, R^{7e}, R^{7f}, R^{7g}, and R^{7h} are H;
(c) at least three of R^{7a}, R^{7b}, R^{7c}, R^{7d}, R^{7e}, R^{7f}, R^{7g}, and R^{7h} are H;
(d) at least four of R^{7a}, R^{7b}, R^{7c}, R^{7d}, R^{7e}, R^{7f}, R^{7g}, and R^{7h} are H;
(e) at least five of R^{7a}, R^{7b}, R^{7c}, R^{7d}, R^{7e}, R^{7f}, R^{7g}, and R^{7h} are H;
(f) at least six of R^{7a}, R^{7b}, R^{7c}, R^{7d}, R^{7e}, R^{7f}, R^{7g}, and R^{7h} are H;
(g) at least seven of R^{7a}, R^{7b}, R^{7c}, R^{7d}, R^{7e}, R^{7f}, R^{7g}, and R^{7h} are H;
(h) each of R^{7a}, R^{7b}, R^{7c}, R^{7d}, R^{7e}, R^{7f}, R^{7g}, and R^{7h} are H;
(i) at least one of R^{8a}, R^{8b}, R^{8c}, and R^{8d} is H;
(j) at least two of R^{8a}, R^{8b}, R^{8c}, and R^{8d} are H;
(k) at least three of R^{8a}, R^{8b}, R^{8c}, and R^{8d} are H; and
(l) each of R^{8a}, R^{8b}, R^{8c}, and R^{8d} are H.

15. The compound of claim 1, wherein at least one of the following applies:
(a) L¹ is selected from the group consisting of 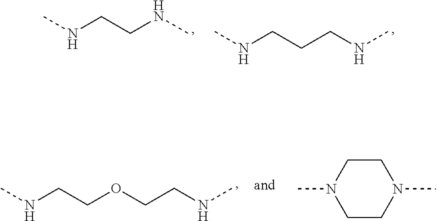
(b) L² is —C(═O)—; and
(c) X is C(CH₃).

16. (canceled)

17. (canceled)

18. The compound of claim 1, which is selected from the group consisting of:
methyl 5-((2-(2-((tert-butoxycarbonyl)amino)acetamido)ethyl)carbamoyl)-2-(2-(4-fluorophenyl)butanamido)-4-methylthiophene-3-carboxylate;
methyl 5-((2-(2-aminoacetamido)ethyl)carbamoyl)-2-(2-(4-fluorophenyl)butanamido)-4-methylthiophene-3-carboxylate;
methyl 5-((2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate;
methyl 5-((2-((S)-2-amino-3-methylbutanamido)ethyl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3-oxa-5,8,11-triazatridecan-13-yl)carbamoyl)-2-(2-(4-fluorophenyl)butanamido)-4-methylthiophene-3-carboxylate;
methyl 5-((2-(2-(2-aminoacetamido)acetamido)ethyl)carbamoyl)-2-(2-(4-fluorophenyl)butanamido)-4-methylthiophene-3-carboxylate;
methyl 5-((2-((S)-2-((S)-2-amino-3-methylbutanamido)-3-methylbutanamido)ethyl)carbamoyl)-2-(2-(4-fluorophenyl)butanamido)-4-methylthiophene-3-carboxylate;
methyl 2-(2-(4-fluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate;
methyl 5-(((5S,8S,11S,14S)-14-amino-5,8,11-triisopropyl-15-methyl-4,7,10,13-tetraoxo-3,6,9,12-tetraazahexadecyl)carbamoyl)-2-(2-(4-fluorophenyl)butanamido)-4-methylthiophene-3-carboxylate;
methyl 5-((2,2-dimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate;
methyl 5-((2-(2-(2-(2-aminoacetamido)acetamido)acetamido)ethyl)carbamoyl)-2-(2-(4-fluorophenyl)butanamido)-4-methylthiophene-3-carboxylate;
methyl 5-((2,2-dimethyl-4,7,10,13,16-pentaoxo-3-oxa-5,8,11,14,17-pentaazanonadecan-19-yl)carbamoyl)-2-(2-(4-fluorophenyl)butanamido)-4-methylthiophene-3-carboxylate;
methyl 5-((14-amino-4,7,10,13-tetraoxo-3,6,9,12-tetraazatetradecyl)carbamoyl)-2-(2-(4-fluorophenyl)butanamido)-4-methylthiophene-3-carboxylate;
methyl 5-((2-((S)-2-amino-4-methylpentanamido)ethyl)carbamoyl)-2-(2-(4-fluorophenyl)butanamido)-4-methylthiophene-3-carboxylate;
methyl 5-((2-((S)-2-((S)-2-amino-4-methylpentanamido)-4-methylpentanamido)ethyl)carbamoyl)-2-(2-(4-fluorophenyl)butanamido)-4-methylthiophene-3-carboxylate;
methyl 5-((2-((S)-2-((S)-2-((S)-2-amino-4-methylpentanamido)-4-methylpentanamido)-4-methylpentanamido)ethyl)carbamoyl)-2-(2-(4-fluorophenyl)butanamido)-4-methylthiophene-3-carboxylate;
methyl 5-((2-((R)-2-amino-3-methylbutanamido)ethyl)carbamoyl)-2-(2-(4-fluorophenyl)butanamido)-4-methylthiophene-3-carboxylate;
methyl 5-((2-((R)-2-((R)-2-amino-3-methylbutanamido)-3-methylbutanamido)ethyl)carbamoyl)-2-(2-(4-fluorophenyl)butanamido)-4-methylthiophene-3-carboxylate;
methyl 5-((2-((R)-2-((R)-2-((R)-2-amino-3-methylbutanamido)-3-methylbutanamido)-3-methylbutanamido)ethyl)carbamoyl)-2-(2-(4-fluorophenyl)butanamido)-4-methylthiophene-3-carboxylate;
methyl 5-((2-((S)-2-((S)-2-((S)-2-amino-3-phenylpropanamido)-3-methylbutanamido)-3-methylbutanamido)ethyl)carbamoyl)-2-(2-(4-fluorophenyl)butanamido)-4-methylthiophene-3-carboxylate;
methyl 5-((2-((S)-2-((S)-2-(2-aminoacetamido)-3-methylbutanamido)-3-methylbutanamido)ethyl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3-oxa-5,8,11-triazatridecan-13-yl)carbamoyl)-4-methyl-2-(2-(4-(trifluoromethyl)phenyl)butanamido)thiophene-3-carboxylate;
methyl 5-((2-((S)-2-((S)-2-amino-3-methylbutanamido)-3-methylbutanamido)ethyl)carbamoyl)-4-methyl-2-(2-(4-(trifluoromethyl)phenyl)butanamido)thiophene-3-carboxylate;
methyl 4-methyl-2-(2-(4-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 4-methyl-5-(((6S,9S,12S,15S)-6,9,12,15-tetraisopropyl-2,2-dimethyl-4,7,10,13,16-pentaoxo-3-oxa-5,8,11,14,17-pentaazanonadecan-19-yl)carbamoyl)-2-(2-(4-(trifluoromethyl)phenyl)butanamido)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(4-(trifluoromethyl)phenyl)butanamido)thiophene-3-carboxylate;
methyl 5-(((5S,8S,11S,14S)-14-amino-5,8,11-triisopropyl-15-methyl-4,7,10,13-tetraoxo-3,6,9,12-tetraazahexadecyl)carbamoyl)-4-methyl-2-(2-(4-(trifluoromethyl)phenyl)butanamido)thiophene-3-carboxylate;
methyl 4-methyl-5-(((6S,9S)-2,2,6,9-tetramethyl-4,7,10-trioxo-3-oxa-5,8,11-triazatridecan-13-yl)carbamoyl)-2-(2-(4-(trifluoromethyl)phenyl)butanamido)thiophene-3-carboxylate;
methyl 5-((2-((S)-2-((S)-2-aminopropanamido)propanamido)ethyl)carbamoyl)-4-methyl-2-(2-(4-(trifluoromethyl)phenyl)butanamido)thiophene-3-carboxylate;
methyl 2-(2-(4-fluorophenyl)butanamido)-4-methyl-5-(piperazine-1-carbonyl)thiophene-3-carboxylate;
tert-butyl (2-(5-carbamoyl-2-(2-(4-fluorophenyl)butanamido)-4-methylthiophene-3-carboxamido)ethyl)carbamate;
methyl 5-(4-((tert-butoxycarbonyl)-L-valyl-L-valyl)piperazine-1-carbonyl)-2-(2-(4-fluorophenyl)butanamido)-4-methylthiophene-3-carboxylate;
methyl 5-(4-(L-valyl-L-valyl)piperazine-1-carbonyl)-2-(2-(4-fluorophenyl)butanamido)-4-methylthiophene-3-carboxylate;
N⁴-(2-aminoethyl)-5-(2-(4-fluorophenyl)butanamido)-3-methylthiophene-2,4-dicarboxamide;
tert-butyl ((2S)-1-(((2S)-1-((2-(5-carbamoyl-2-(2-(4-fluorophenyl)butanamido)-4-methylthiophene-3-carboxamido)ethyl)amino)-3-methyl-1-oxobutan-2-yl)amino)-3-methyl-1-oxobutan-2-yl)carbamate;
N⁴-(2-((S)-2-((S)-2-amino-3-methylbutanamido)-3-methylbutanamido)ethyl)-5-(2-(4-fluorophenyl)butanamido)-3-methylthiophene-2,4-dicarboxamide;
methyl 2-(2-(4-fluorophenyl)butanamido)-4-methyl-5-(((5S,8S,11S)-5,8,11-triisopropyl-4,7,10,13-tetraoxo-3,6,9,12-tetraazatetradecyl)carbamoyl)thiophene-3-carboxylate;
methyl 5-(4-(L-valyl-L-valyl-L-valyl)piperazine-1-carbonyl)-2-(2-(4-fluorophenyl)butanamido)-4-methylthiophene-3-carboxylate;
tert-butyl ((7S,10S,13S)-1-(5-carbamoyl-2-(2-(4-fluorophenyl)butanamido)-4-methylthiophen-3-yl)-7,10-diisopropyl-14-methyl-1,6,9,12-tetraoxo-2,5,8,11-tetraazapentadecan-13-yl)carbamate;
methyl 5-((3-((tert-butoxycarbonyl)amino)propyl)carbamoyl)-2-(2-(4-fluorophenyl)butanamido)-4-methylthiophene-3-carboxylate;
methyl 5-((3-aminopropyl)carbamoyl)-2-(2-(4-fluorophenyl)butanamido)-4-methylthiophene-3-carboxylate;
methyl 5-((2-(2-((tert-butoxycarbonyl)amino)ethoxy)ethyl)carbamoyl)-2-(2-(4-fluorophenyl)butanamido)-4-methylthiophene-3-carboxylate;
methyl 2-(2-(4-fluorophenyl)butanamido)-5-(((6S,9S)-6-isopropyl-2,2,9-trimethyl-4,7,10-trioxo-3-oxa-5,8,11-triazatridecan-13-yl)carbamoyl)-4-methylthiophene-3-carboxylate;
methyl 5-((2-((S)-2-((S)-2-amino-3-methylbutanamido)propanamido)ethyl)carbamoyl)-2-(2-(4-fluorophenyl)butanamido)-4-methylthiophene-3-carboxylate;
methyl 5-(((6S,9S,12S)-6,9-diisopropyl-2,2,12-trimethyl-4,7,10,13-tetraoxo-3-oxa-5,8,11,14-tetraazahexadecan-16-yl)carbamoyl)-2-(2-(4-fluorophenyl)butanamido)-4-methylthiophene-3-carboxylate;
methyl 5-(((6S,9S)-6,9-diisopropyl-2,2-dimethyl-4,7,10-trioxo-3,14-dioxa-5,8,11-triazahexadecan-16-yl)carbamoyl)-2-(2-(4-fluorophenyl)butanamido)-4-methylthiophene-3-carboxylate;
methyl 5-((3-((S)-2-((S)-2-amino-3-methylbutanamido)-3-methylbutanamido)propyl)carbamoyl)-2-(2-(4-fluorophenyl)butanamido)-4-methylthiophene-3-carboxylate;
methyl 5-((2-(2-((S)-2-((S)-2-amino-3-methylbutanamido)-3-methylbutanamido)ethoxy)ethyl)carbamoyl)-2-(2-(4-fluorophenyl)butanamido)-4-methylthiophene-3-carboxylate;
methyl 2-(2-(benzo[d][1,3]dioxol-5-yl)butanamido)-5-carbamoyl-4-methylthiophene-3-carboxylate;
2-(tert-butyl) 4-methyl 5-(2-(benzo[d][1,3]dioxol-5-yl)butanamido)-3-methylthiophene-2,4-dicarboxylate;
2-(tert-butyl) 4-methyl 5-(2-(3,5-difluorophenyl)butanamido)-3-methylthiophene-2,4-dicarboxylate;
methyl 5-carbamoyl-2-(2-(3,5-difluorophenyl)butanamido)-4-methylthiophene-3-carboxylate;
methyl 5-carbamoyl-2-(2-(4-cyanophenyl)butanamido)-4-methylthiophene-3-carboxylate;
methyl 5-((2-aminoethyl)carbamoyl)-2-(2-(benzo[d][1,3]dioxol-5-yl)butanamido)-4-methylthiophene-3-carboxylate;
methyl 2-(2-(benzo[d][1,3]dioxol-5-yl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(benzo[d][1,3]dioxol-5-yl)butanamido)-4-methylthiophene-3-carboxylate;
methyl 5-((2-aminoethyl)carbamoyl)-2-(2-(3,5-difluorophenyl)butanamido)-4-methylthiophene-3-carboxylate;
methyl 2-(2-(3,5-difluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(3,5-difluorophenyl)butanamido)-4-methylthiophene-3-carboxylate;
methyl 5-((2-aminoethyl)carbamoyl)-2-(2-(4-cyanophenyl)butanamido)-4-methylthiophene-3-carboxylate;
methyl 2-(2-(4-cyanophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(4-cyanophenyl)butanamido)-4-methylthiophene-3-carboxylate;
methyl 2-(2-(4-fluorophenyl)butanamido)-4-methyl-5-(((6S,9S,12S)-6,9,12-triisopropyl-2,2-dimethyl-4,7,10,13-tetraoxo-3,17-dioxa-5,8,11,14-tetraazanonadecan-19-yl)carbamoyl)thiophene-3-carboxylate;
methyl 5-(((8S,11S,14S)-14-amino-8,11-diisopropyl-15-methyl-7,10,13-trioxo-3-oxa-6,9,12-triazahexadecyl)carbamoyl)-2-(2-(4-fluorophenyl)butanamido)-4-methylthiophene-3-carboxylate;
2-(tert-butyl) 4-methyl 5-(2-(3,4-difluorophenyl)butanamido)-3-methylthiophene-2,4-dicarboxylate;
2-(tert-butyl) 4-methyl 3-methyl-5-(2-(m-tolyl)butanamido)thiophene-2,4-dicarboxylate;
2-(tert-butyl) 4-methyl 3-methyl-5-(2-(3-(trifluoromethyl)phenyl)butanamido)thiophene-2,4-dicarboxylate;
methyl 5-carbamoyl-2-(2-(3,4-difluorophenyl)butanamido)-4-methylthiophene-3-carboxylate;
methyl 5-carbamoyl-4-methyl-2-(2-(m-tolyl)butanamido)thiophene-3-carboxylate;
methyl 5-carbamoyl-4-methyl-2-(2-(3-(trifluoromethyl)phenyl)butanamido)thiophene-3-carboxylate;
methyl 5-((2-aminoethyl)carbamoyl)-2-(2-(3,4-difluorophenyl)butanamido)-4-methylthiophene-3-carboxylate;
methyl 2-(2-(3,4-difluorophenyl)butanamido)-4-methyl-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-2-(2-(3,4-difluorophenyl)butanamido)-4-methylthiophene-3-carboxylate;
methyl 5-((2-aminoethyl)carbamoyl)-4-methyl-2-(2-(m-tolyl)butanamido)thiophene-3-carboxylate;
methyl 4-methyl-2-(2-(m-tolyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(m-tolyl)butanamido)thiophene-3-carboxylate;
methyl 5-((2-aminoethyl)carbamoyl)-4-methyl-2-(2-(3-(trifluoromethyl)phenyl)butanamido)thiophene-3-carboxylate;
methyl 4-methyl-2-(2-(3-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(3-(trifluoromethyl)phenyl)butanamido)thiophene-3-carboxylate;
methyl 5-(((R)-1-(tert-butoxy)-3-methyl-1-oxobutan-2-yl)carbamoyl)-2-(2-(4-fluorophenyl)butanamido)-4-methylthiophene-3-carboxylate;
(5-(2-(4-fluorophenyl)butanamido)-4-(methoxycarbonyl)-3-methylthiophene-2-carbonyl)-D-valine;
2-(tert-butyl) 4-methyl 3-methyl-5-(2-(o-tolyl)butanamido)thiophene-2,4-dicarboxylate;
methyl 5-carbamoyl-4-methyl-2-(2-(o-tolyl)butanamido)thiophene-3-carboxylate;
methyl 5-((2-aminoethyl)carbamoyl)-4-methyl-2-(2-(o-tolyl)butanamido)thiophene-3-carboxylate;
methyl 4-methyl-2-(2-(o-tolyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(o-tolyl)butanamido)thiophene-3-carboxylate;
2-(tert-butyl) 4-methyl 3-methyl-5-(2-(2-(trifluoromethyl)phenyl)butanamido)thiophene-2,4-dicarboxylate;
methyl 5-carbamoyl-4-methyl-2-(2-(2-(trifluoromethyl)phenyl)butanamido)thiophene-3-carboxylate;
methyl 4-methyl-2-(2-(2-(trifluoromethyl)phenyl)butanamido)-5-(((6S,9S,12S)-6,9,12-triisopropyl-2,2-dimethyl-4,7,10,13-tetraoxo-3-oxa-5,8,11,14-tetraazahexadecan-16-yl)carbamoyl)thiophene-3-carboxylate;
methyl 5-((2-((S)-2-((S)-2-((S)-2-amino-3-methylbutanamido)-3-methylbutanamido)-3-methylbutanamido)ethyl)carbamoyl)-4-methyl-2-(2-(2-(trifluoromethyl)phenyl)butanamido)thiophene-3-carboxylate;
methyl 5-(((R)-1-(((R)-1-(tert-butoxy)-3-methyl-1-oxobutan-2-yl)amino)-3-methyl-1-oxobutan-2-yl)carbamoyl)-2-(2-(4-fluorophenyl)butanamido)-4-methylthiophene-3-carboxylate; and
(5-(2-(4-fluorophenyl)butanamido)-4-(methoxycarbonyl)-3-methylthiophene-2-carbonyl)-D-valyl-D-valine.

19. A pharmaceutical composition comprising at least one compound of claim 1 and at least one pharmaceutically acceptable excipient.

20. A method of treating, ameliorating, or preventing an orthopoxvirus infection in a subject in need thereof, the method comprising administering to the subject a therapeutically effective amount of the compound of claim 1 or a pharmaceutical composition thereof.

21. The method of claim 20, wherein the orthopoxvirus is selected from the group consisting of Molluscum contagiosum virus (MCV), camelpox virus, cowpox virus, mousepox virus, horsepox virus, monkeypox virus, raccoonpox virus, tanapox virus, variola (smallpox) virus, Yoka poxvirus, cervidpoxvirus (deerpox), avipoxvirus (fowlpox), capripoxvirus (goatpox), leporipoxvirus (myxoma virus), parapoxvirus (orf virus), suipoxvirus (swinepox), and yatapoxvirus (Yaba-like disease virus).

22. The method of claim 21, wherein at least one of the following applies:
(a) folding and/or function of processivity factor mD4 is inhibited in the orthopoxvirus;
(b) DNA polymerase processivity is disrupted in the orthopoxvirus; and
(c) the orthopoxvirus infection is caused by a MCV.

23. (canceled)

24. (canceled)

25. The method of claim 20, wherein the subject is a mammal, optionally wherein the mammal is a human.

26. (canceled)

27. The method of claim 20, wherein the administration is topical, optionally wherein the topical administration is to at least one lesion associated with the orthopoxvirus infection.

28. (canceled)
